SEQUENCE LISTING (652 sequences)

SEQ ID NO: 1 - DNA, 2577 bp, Mouse
atgccagctttggctatcatgggtctcagcctggctgctttcctggagcttgggatgggggcctctttgtgtctgtcacagcaattcaaggcacaaggggactacatactgggcgggctatttcccctgggctcaaccgaggaggccactctcaaccagagaacacaacccaacagcatcccgtgcaacaggttctcaccccttggtttgttcctggccatggctatgaagatggctgtggaggagatcaacaatggatctgccttgctccctgggctgcggctgggctatgacctatttgacacatgctccgagccagtggtcaccatgaaatccagtctcatgttcctggccaaggtgggcagtcaaagcattgctgcctactgcaactacacacagtaccaaccccgtgtgctggctgtcatcggcccccactcatcagagcttgccctcattacaggcaagttcttcagcttcttcctcatgccacaggtcagctatagtgccagcatggatcggctaagtgaccgggaaacgtttccatccttcttccgcacagtgcccagtgaccgggtgcagctgcaggcagttgtgactctgttgcagaacttcagctggaactgggtggccgccttagggagtgatgatgactatggccgggaaggtctgagcatcttttctagtctggccaatgcacgaggtatctgcatcgcacatgagggcctggtgccacaacatgacactagtggccaacagttgggcaaggtgctggatgtactacgccaagtgaaccaaagtaaagtacaagtggtggtgctgtttgcctctgcccgtgctgtctactccctttttagttacagcatccatcatggcctctcacccaaggtatgggtggccagtgagtcttggctgacatctgacctggtcatgacacttcccaatattgcccgtgtgggcactgtgcttgggtttttgcagcggggtgccctactgcctgaattttcccattatgtggagactcaccttgccctggccgctgacccagcattctgtgcctcactgaatgcggagttggatctggaggaacatgtgatggggcaacgctgtccacggtgtgacgacatcatgctgcagaacctatcatctgggctgttgcagaacctatcagctgggcaattgcaccaccaaatatttgcaacctatgcagctgtgtacagtgtggctcaagcccttcacaacaccctacagtgcaatgtctcacattgccacgtatcagaacatgttctaccctggcagctcctggagaacatgtacaatatgagtttccatgctcgagacttgacactacagtttgatgctgaagggaatgtagacatggaatatgacctgaagatgtgggtgtggcagagccctacacctgtattacatactgtgggcaccttcaacggcacccttcagctgcagcagtctaaaatgtactggccaggcaaccaggtgccagtctcccagtgttcccgccagtgcaaagatggccaggttcgccgagtaaagggctttcattcctgctgctatgactgcgtggactgcaaggcgggcagctaccggaagcatccagatgacttcacctgtactccatgtaaccaggaccagtggtccccagagaaaagcacagcctgcttacctcgcaggcccaagtttctggcttggggggagccagttgtgctgtcactcctcctgctgctttgcctggtgctgggtctagcactggctgctctggggctctctgtccaccactgggacagccctcttgtccaggcctcaggtggctcacagttctgctttggcctgatctgcctaggcctcttctgcctcagtgtccttctgttcccagggcggccaagctctgccagctgccttgcacaacaaccaatggctcacctccctctcacaggctgcctgagcacactcttcctgcaagcagctgagacctttgtggagtctgagctgccactgagctgggcaaactggctatgcagctaccttcggggactctgggcctggctagtggtactgttggccacttttgtggaggcagcactatgtgcctggtatttgatcgctttcccaccagaggtggtgacagactggtcagtgctgcccacagaggtactggagcactgccacgtgcgttcctgggtcagcctgggcttggtgcacatcaccaatgcaatgttagctttcctctgctttctgggcactttcctggtacagagccagcctggccgctacaaccgtgcccgtggtctcaccttcgccatgctagcttatttcatcacctgggtctcttttgtgcccctcctggccaatgtgcaggtggcctaccagccagctgtgcagatgggtgctatcctagtctgtgccctgggcatcctggtcaccttccacctgcccaagtgctatgtgcttctttggctgccaaagctcaacacccaggagttcttcctgggaaggaatgccaagaaagcagcagatgagaacagtggcggtggtgaggcagctcagggacacaatgaatga

SEQ ID NO: 2 - DNA, 11809 bp, Mouse
atctgagccttagacacagcactggtgccaggcaaacactcctgggcctacatgcttggggcctcttcatattccaaaagctgtctttgggtaagatgaagttcctctggcagtggcatgagtgctgaaggctctttccctgcccttcacctgctttcttgatagtctctctgcataccaaacaggcccttgtctcctgggaaatggaaactatgaaatcaatagctgaggcttctctaggaaagcctgccctggtcagtacaacctgtttcacagcttctatagaatagttacatcagccttctgaagatggcctcttagagcacatgcacccccaagattctaagatgtcaatactaactgaccaaaccatacctctctagccagccctgctgctcctgttgtctggtacccaggtgactgaggacatgactggtggaaggaaactaggcccctttgtctgtcagatggccatacccagcatggctgatgcccagtgtataagaccctacgcttttccactggtcttaatgttaaaccctaggacagtgtcctcagcatagctggtgtgtgtgaatgcaaactttggggcatatctcttccattaagcactgtgatatatgtagtatttccaacaaataaattatacctacatgattgggtatagcattctgggatgggtcacaggtgtgtcaggtgcctaattatgtgggggaagaacatagaaatatataggtggggagggagctaaccctaggaataaggctaaagcatgtgtctccagtcctgaagactcaaagggcaacgtgaatcatgagacatgttcaggactgaaggagttgccatgtatctgtccttgatgtatcttaatcatacatacactatgagatctgtgttacctccattttgcaggtgagaaaagaaacacctgaatggcctaccttaaagggctaagtgggaaaataggtctgaagataacccaggcactgtgtgacaaagcgggaagaaaactagagatgctttcttcatggcaacaacctagagggtacaacctagtggtttcttcttggtactccactgtatacaccccatctgcttgggctgtacattgtctgaccatgcttataacaaaagtcacatactactagccaagactgagaacttagagcgactggccagaaagtaaagatacaacagttgatatgtgtgccacacacagatccatgtgtacatgtctattaattatgtgaacgtgctttgtggacatcctcacaaagcagcagggaaatgcaaaggtcatttccataacacctgctggacaccatatgacattgagattaccggggtgcccattccaacaagagttaatagctccccctatgtttgggtgccagaaacctgatttgttagcaatagctccctcacatccagattaagagggggatggcttagctagggttactatgatgaaactatgaccaaagcaacttgtgggtaaaagggtgtatttggcttacacttccatatcacttcatcaaagtgaggacaggaactcaaatagagtaggaatttggtgacaagagctgatgtagaggcaatgcagtggtgccacttagtggcgcgctcagtctgctccctttcttaatagaatgcaagaccaccagcccatgggtggcaccacaatgggaccgggcccttccccatcggtcactaagaaaatgccctacagccagatcttatggagacattttctcaacggaggctcactcctttcagataactctatatcaaattgacataaaccagaacagaggaggaggctaagaaggaaactgccaattgcatacatgcacacacctggccctagcagctgcaggaagctatttgtttatggccttttctcattttcatggaccagcatgagcactctgcagagagagatgcctgcatgcctgccaaggcaggagtgcttacactgaaggtcaacaggatggcaggggggctgcagagcttccaagtgtcagaaccccagcagaagagctgagacccttgcccgaggactcaggcgggttgggaaggccaggaaattcagccagagctcttcttcagatggggtaccatctgaaggttagaccagctagccagctgttgttgagggaccacctctgcagcccctacctttggaagatagaaagtgtctctgtgacaagtatggccattgtgcccccttattccacagtcaacagaaaccctggaatcctgaacacttctgcagcttcttttttacagtctgccaggttgctctaggaatgaagggtgccgagaggcttgggcgtaggcaggtgacaagaccacagttagtggtcacagctggcttactggatcactcttggacagagtttgttagatatggagtggagtatacacaaggcatcaggcgggggatattgaatgtatcaccggagctccttggggcttggcagccaagcacagcagtggttttgctaaacaaatccacggttccctccccttgacgcagtacatctgtggctccaaccccacacacccacccattgttagtgctggagacttctacctaccatgccagctttggctatcatgggtctcagcctggctgctttcctggagcttgggatgggggcctctttgtgtctgtcacagcaattcaaggcacaaggggactacatactgggcgggctatttcccctgggctcaaccgaggaggccactctcaaccagagaacacaacccaacagcatcccgtgcaacaggtatggaggctagtagctggggtgggagtgaaccgaagcttggcagctttggctccgtggtactaccaatctgggaagaggtggtgatcagtttccatgtggcctcaggttctcaccccttggtttgttcctggccatggctatgaagatggctgtggaggagatcaacaatggatctgccttgctccctgggctgcggctgggctatgacctatttgacacatgctccgagccagtggtcaccatgaaatccagtctcatgttcctggccaaggtgggcagtcaaagcattgctgcctactgcaactacacacagtaccaaccccgtgtgctggctgtcatcggcccccactcatcagagcttgccctcattacaggcaagttcttcagcttcttcctcatgccacaggtgagcccacttcctttgtgttctcaaccgattgcacccattgagctctcatatcagaaagtgcttcttgatcaccacaggtcagctatagtgccagcatggatcggctaagtgaccgggaaacgtttccatccttcttccgcacagtgcccagtgaccgggtgcagctgcaggcagttgtgactctgttgcagaacttcagctggaactgggtggccgccttagggagtgatgatgactatggccgggaaggtctgagcatcttttctagtctggccaatgcacgaggtatctgcatcgcacatgagggcctggtgccacaacatgacactagtggccaacagttgggcaaggtgctggatgtactacgccaagtgaaccaaagtaaagtacaagtggtggtgctgtttgcctctgcccgtgctgtctactccctttttagttacagcatccatcatggcctctcacccaaggtatgggtggccagtgagtcttggctgacatctgacctggtcatgacacttcccaatattgcccgtgtgggcactgtgcttgggtttttgcagcggggtgccctactgcctgaattttcccattatgtggagactcaccttgccctggccgctgacccagcattctgtgcctcactgaatgcggagttggatctggaggaacatgtgatggggcaacgctgtccacggtgtgacgacatcatgctgcagaacctatcatctgggctgttgcagaacctatcagctgggcaattgcaccaccaaatatttgcaacctatgcagctgtgtacagtgtggctcaagcccttcacaacaccctacagtgcaatgtctcacattgccacgtatcagaacatgttctaccctggcaggtaagggtagggttttttgctgggttttgcctgctcctgcaggaacactgaaccaggcagagccaaatcttgttgtgactggagaggccttaccctgactccactccacagctcctggagaacatgtacaatatgagtttccatgctcgagacttgacactacagtttgatgctgaagggaatgtagacatggaatatgacctgaagatgtgggtgtggcagagccctacacctgtattacatactgtgggcaccttcaacggcacccttcagctgcagcagtctaaaatgtactggccaggcaaccaggtaaggacaagacaggcaaaaaggatggtgggtagaagcttgtcggtcttgggccagtgctagccaaggggaggcctaacccaaggctccatgtacaggtgccagtctcccagtgttcccgccagtgcaaagatggccaggttcgccgagtaaagggctttcattcctgctgctatgactgcgtggactgcaaggcgggcagctaccggaagcatccaggtgaaccgtcttccctagacagtctgcacagccgggctagggggcagaagcattcaagtctggcaagcgccctcccgcggggctaatgtggagacagttactgtgggggctggctggggaggtcggtctcccatcagcagaccccacattacttttcttccttccatcactacagatgacttcacctgtactccatgtaaccaggaccagtggtccccagagaaaagcacagcctgcttacctcgcaggcccaagtttctggcttggggggagccagttgtgctgtcactcctcctgctgctttgcctggtgctgggtctagcactggctgctctggggctctctgtccaccactgggacagccctcttgtccaggcctcaggtggctcacagttctgctttggcctgatctgcctaggcctcttctgcctcagtgtccttctgttcccagggcggccaagctctgccagctgccttgcacaacaaccaatggctcacctccctctcacaggctgcctgagcacactcttcctgcaagcagctgagacctttgtggagtctgagctgccactgagctgggcaaactggctatgcagctaccttcggggactctgggcctggctagtggtactgttggccacttttgtggaggcagcactatgtgcctggtatttgatcgctttcccaccagaggtggtgacagactggtcagtgctgcccacagaggtactggagcactgccacgtgcgttcctgggtcagcctgggcttggtgcacatcaccaatgcaatgttagctttcctctgctttctgggcactttcctggtacagagccagcctggccgctacaaccgtgcccgtggtctcaccttcgccatgctagcttatttcatcacctgggtctcttttgtgcccctcctggccaatgtgcaggtggcctaccagccagctgtgcagatgggtgctatcctagtctgtgccctgggcatcctggtcaccttccacctgcccaagtgctatgtgcttctttggctgccaaagctcaacacccaggagttcttcctgggaaggaatgccaagaaagcagcagatgagaacagtggcggtggtgaggcagctcagggacacaatgaatgaccactgacccgtgaccttccctttagggaacctagccctaccagaaatctcctaagccaacaagccccgaatagtacctcagcctgagacgtgagacacttaactatagacttggactccactgaccttagcctcacagtgaccccttccccaaacccccaaggcctgcagtgcacaagatggaccctatgagcccacctatcctttcaaagcaagattatccttgatcctattatgcccacctaaggcctgcccaggtgacccacaaaaggttctttgggacttcatagccatactttgaattcagaaattccccaggcagaccatgggagaccagaaggtactgcttgcctgaacatgcccagccctgagccctcactcagcaccctgtccaggcgtcccaggaatagaaggctgggcatgtatgtgtgtgtgtgtgtgtgtgtgtgtgtgtgtgtgtgtgtgtgtatgtacgtatgtatgtatgtatcaggacagaacaagaaagacatcaggcagaggacactcaggaggtaggcaacatccagccttctccatccctagctgagccctagcctgtaggagagaaccaggtcgccgccagcaccttggacagatcacacacagggtgcgggtcagcaccacggccagcgccagccacgcgggacccctggaatcagcttctagtaccaaggacagaaaagttgccgcaaggccccttactggccagcaccagggacagagccacatgcctaagcggcaagggacaagagcatcgtccatctgcaggcaggatcagacccgggtcagttctggactggcccccacacctgaatcccggagcagctcagctggagaaaagagaaacaagccacacatcagtcccataaaattaaacgctttttttagtgtttaaaatagcatttacacagaagcagcatttacacagaagcagctctatgtcaactacccagtcactcagactttgacacagtgtctagtgtagatgtgtggggccgctgtgccgggatggcagtggcacatgatgatgggcagccaccagaacagaaacagaacagggcccagctctgcagctcttgtgttcactgtcacccaccactgagactgagacagtggctaggtgccaggtctctctcctgtctctcctactagctacccttcacataccttcagtacaaactgtgttgtcatgtgccaagtagcaggtggggaaaggggcatgcaaactgcccctttgggtaactagctgccacccttagagcaggcaggctagcaataaataaataagttagaccccacctgggcagccagagaggtttgaaggctctgtctaacccctcaaaaatcccaccttggcctgacaggtgaggcccatgaacttagcgacagtcagcctgtgtccctgtgcacagttctgtgaggctttggggcaaggggtaccaagagcccaagagagcctttcttgttctaaatggaggtcacttccaaagaagggaaccaggaggtggtccctgagacttgtgctgaggacttaaagtcagagatgtctccttacaagactctatagatacttgagctgtaccaccatcagcagccccaagagcagacaaaatgtcaagccaatatcctggtggtatggctgccctcaggccctcctctgtagcctgctccctctgccctggcccagagcccacagctgatctatcctggctggccaccaccacggccagcgcagagctcctggcacagcaggagcacagactcagccacaggcagcgctgaagacattggttgatcatcacatgatgtccacaaagaactcacaggggtttcccatggccttttggaaggactggcggctacctgtaagttctggagggacagcagccagctcccggacgggtggccctccaggtggcccacccactactgcataggcctttgtaagggggtgcagtggggggagccctggggcaacagctgaagcctgacttcgagggctactgccacggctaagctggctgacaggccgctcccaccagccggtgctaccagacccacttggtactgtgtggtctgattcactgccactacccccagctccagttgcccggcgctcctctcggcctggggtccgatggctgctccgtgtggacccactgctcttgctccctagggggagggaaggggacaacagagtcagcacgaggcctggccacttccagggccaccagctgctcccagacagtcagggcaggacctggtaagcctggagatggtaggggaatggcagccatgcagataccaggaacagctgagaggcgagaagctaggggcagtggcagacagcagggacaacaggggccagcctggcaccccacacctaaccccaatgcttgaaccaagggttaatgttacagctgagaaactaaaaaccagcgaaggccctgtgtgcccagcattcccattagccatcctgggttcaccacccaaagacccaaccagggtccacccaaccccaggaccctggtcatctaatttgcttagcccctgtcctgaaagtagtgggaacctgaaaacacgtgctggctggggacatgctgagagggacacagggggacctggcttaccggcccgagagtccactctgctagtccttcagtctaaggcttgctcagcacaaagcaagggatagcacaagtcacacaccagtccagtgctcaccaatggctaataggacgattttgggccaagctgagcctgggtacatgcaagggcctgtccatggtcaggattcactcgatagcttccccttgggctttgccaccctctggcccaacctctcctgagtctttctctggaccttgtagcacaagtgtgccccactctgcctaagacctccacatcagtccatctcctcctgagggacacccacccttcaagatcttcaatatccctgggatatgctttaacactgatatgctttaacagtgttgcttgatactcttatctggcactctgttgggatgcaggctccataactgataaagcccattctccccctagcttggggcctagagagtgcccctacctgctatcagtggttactttcattcttgccatatcatctcctggcctcttgcctctgccacctagcacaccaggctgtcttcctattctctaacggcttctacccacatcagcccctccctgtcccacacactgactcttgagatggaacccaccgggactcaaacacacagcaggagcacagagggaagcgtcggggccaggcagagcgtgggagtgggagggagtgggaggaggggtggcacgcctctcaccttcactctgctggctcccagcactgccgctgccgcagctgaagccagggtcctggtaagcaggcgggaagcagggcgggggtcctgggtactggtaggggtagccttgacccaagggccagggtactgatgggtggggcagtggggccagtgtgtcctgatctgaggctccactggagccactgttgaggttcagggatgcgaggtctggcagggagggagggagggaggggtaagtgaaggcaaatgaatgaggccacagcaaccctacccaaccgcacccctactcactactgcacaggtcgccaaagacatagtagcactgctcagaaaaggtgatcttgttcacggtgtgcctcaggaaaccgtgcttcagcatactgctggcatactttcttgcctcccttcgctccttgaagccctccacgtgtgtgtacagccagtccaccacatccgcccctggccacaggtccatcaaagtcagggtagctgagccctgggaagctacgccagaatgaggaacagacggggcccttcccacacagccagggactcaccaatgacagcattggcaatggtgatcttaagccacatgcggtcccggatctccagtcctgagtctggcaactgcatgacgcggacaatggcactcatgtcactcttcacagtcagcggtgcctcctcaagctctgcagagcacacttccctgagcccaggctcacagcgtgaacctccatggggttgagagcaggggccagggtcaaacctcttatctcccatccttgggagatgcccctcatcgaaacttgagctaagaccgggagattcttccccgtcccacagtgcaagtccacgtaggcaaggcagcccccctcccctccccggagagaacaagctgttagctatgttaggtagcagaaaagcaaagcagaggctgccatgtcctcccaattcccccctccgcacaggcctggcaggaccctcaattcatgcagatgaccagtatggccaggcctggagggatatgtacatgtatctttgtgtacacatttgtgaaggtgttggaagcaaacaaaaccttcatatgtaatgggcccctgtaatagctctgatgagcaccaaagctcaaagctagaactgaccattgtccttcaacctcagtttccttgggtgggggggggtcctgtgagctgccacttacgtggggcgccaggcactgagctggttagtgaggaagagctggtgcgtgtgatggcgctggagcagggactcgtaccatagcggggcagggcacccgtcagtgctgctgtgtgggacagccaggcagccgggtcgatgggtcgcactgggtcagctgcatagtttccacagcaacggattacaggtggtaagtaggggggcagcacagaggcagacaagaaagacccccagactgaacacagaaaccccaccctaccccacctttccatggggtaactcaccccttgggatggtgaagtagctccgaggggttgggtcccagcacttggccactgtgagactgatgggcctacagagttgagcagaccatgttgtaagtgaggcccgcacagcccctcccatcctgtgccactcccacccccacttggctcccacctcaccctgtctgggacacgatctcccgaagcacccgtacagcgtcgtcattgctcatgttctcaaagttgacatcgttcacctacggggtttgtggggtcaggggttggtggtgggatgtgggtgcctcttgtccccacagtccccacatggctcccacctgcagcaacatgtcgcccggctcaatgcggccatcagcagccacggccccgcccttcatgatggatccaatgtagatgccgccatcaccccggtcgttgctctggcccacgatgctgatgcccaggaagtggtgcctctctgcaggaggggccgtgagcaggcccccaaagctcccgaggctgtacccacccccagcaggcacccacagcccacaaggcctcacccatgttgagagtgacggtgatgatgttcagggacatggtggagtctgtgatgctgctgaaggaggatgcctgcggagggacccagtgaggggctgtgtgggcaccattcagagcagacaccccacccacctgctgcctacccggtctgtctgcctcaagcgctgcttccgacgacggcatttgtgcttccgaactagccgagaggaggtgctctgctctgtggagctgctcagcctgaggcaggagtcagaaaagcacaaacatgtataaccagctcggacgctcaactacaaatctccagcacgtactgacatgtgcacacgtcacccaccggctcgtattgtcctcctcatctgagtcaataaagctgctagattcaagctcactgctcagtacagtggatgcactgtctggaggtagtcccaggtcccgccgccgatcccctctcgggtgcccattggtccgggcagctgtggggacagtagggtgggtacgactgtgggacttcagtcctaacagaatgcgggtggcctgtgcatttcaaagtttatgcagtaactctggggccacaggggctaggagtaccaggctgggacctctacccaaggatcactgcttggaagaatatgtggaatacttccaggcttggagtataccaaagggataccaaggg

SEQ ID NO: 3 - protein, 858 aa, Mouse
MPALAIMGLSLAAFLELGMGASLCLSQQFKAQGDYILGGLFPLGSTEEATLNQRTQPNSIPCNRFSPLGLFLAMAMKMAVEEINNGSALLPGLRLGYDLFDTCSEPVVTMKSSLMFLAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSSLANARGICIAHEGLVPQHDTSGQQLGKVLDVLRQVNQSKVQVVVLFASARAVYSLFSYSIHHGLSPKVWVASESWLTSDLVMTLPNIARVGTVLGFLQRGALLPEFSHYVETHLALAADPAFCASLNAELDLEEHVMGQRCPRCDDIMLQNLSSGLLQNLSAGQLHHQIFATYAAVYSVAQALHNTLQCNVSHCHVSEHVLPWQLLENMYNMSFHARDLTLQFDAEGNVDMEYDLKMWVWQSPTPVLHTVGTFNGTLQLQQSKMYWPGNQVPVSQCSRQCKDGQVRRVKGFHSCCYDCVDCKAGSYRKHPDDFTCTPCNQDQWSPEKSTACLPRRPKFLAWGEPVVLSLLLLLCLVLGLALAALGLSVHHWDSPLVQASGGSQFCFGLICLGLFCLSVLLFPGRPSSASCLAQQPMAHLPLTGCLSTLFLQAAETFVESELPLSWANWLCSYLRGLWAWLVVLLATFVEAALCAWYLIAFPPEVVTDWSVLPTEVLEHCHVRSWVSLGLVHITNAMLAFLCFLGTFLVQSQPGRYNRARGLTFAMLAYFITWVSFVPLLANVQVAYQPAVQMGAILVCALGILVTFHLPKCYVLLWLPKLNTQEFFLGRNAKKAADENSGGGEAAQGHNE

SEQ ID NO: 4 - DNA, 2559 bp, Homo sapiens
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggcctcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccggtgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 5 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALASARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 6 - DNA, 20 bp, Mouse
cactagagctgccaccttcc

SEQ ID NO: 7 - DNA, 20 bp, Mouse
ccctcagcaccactttttgt

SEQ ID NO: 8 - DNA, 20 bp, Mouse
acaaaaagtggtgctgaggg

SEQ ID NO: 9 - DNA, 20 bp, Mouse
caggagacccaaaggatcaa

SEQ ID NO: 10 - DNA, 20 bp, Mouse
gcttcagaaaatcgaggcac

SEQ ID NO: 11 - DNA, 20 bp, Mouse
gcatgggctatgataggtgg

SEQ ID NO: 12 - DNA, 16 bp, Mouse
tgttgatcccacagcg

SEQ ID NO: 13 - DNA, 20 bp, Mouse
caggaaatgtccacttctgc

SEQ ID NO: 14 - DNA, 18 bp, Mouse
tctatcttgcatccagcc

SEQ ID NO: 15 - DNA, 16 bp, Mouse
gtgctgtgactgtgcg

SEQ ID NO: 16 - DNA, 18 bp, Mouse
cgcagcatttatttggag

SEQ ID NO: 17 - DNA, 19 bp, Mouse
ccgaccctttaggagacac

SEQ ID NO: 18 - DNA, 20 bp, Mouse
tgtgacttcctcttccccac

SEQ ID NO: 19 - DNA, 20 bp, Mouse
tgagccactccagatgtcag

SEQ ID NO: 20 - DNA, 20 bp, Mouse
ccaacgtgcagtcaagaaaa

SEQ ID NO: 21 - DNA, 20 bp, Mouse
ccaacgtgcagtcaagaaaa

SEQ ID NO: 22 - DNA, 20 bp, Mouse
cgagagacaaagtggtgctg

SEQ ID NO: 23 - DNA, 20 bp, Mouse
ttatgaaggccctcaccaac

SEQ ID NO: 24 - DNA, 20 bp, Mouse
ccagctcctagaattgcctg

SEQ ID NO: 25 - DNA, 20 bp, Mouse
gcagtctcccgaaacaagtc

SEQ ID NO: 26 - DNA, 20 bp, Mouse
atagaggaatgggtgcgatg

SEQ ID NO: 27 - DNA, 20 bp, Mouse
taccaggaggggtcagtcag

SEQ ID NO: 28 - DNA, 20 bp, Mouse
tacaagcgagctgaccaatg

SEQ ID NO: 29 - DNA, 20 bp, Mouse
ccaatcagctcgagttagcc

SEQ ID NO: 30 - DNA, 20 bp, Mouse
tgccattgtggatgttcact

SEQ ID NO: 31 - DNA, 20 bp, Mouse
gagtccgaggtcggtcaata

SEQ ID NO: 32 - DNA, 20 bp, Mouse
gctggcttctgtaggtcagg

SEQ ID NO: 33 - DNA, 20 bp, Mouse
tatgagggtcaagggtcagg

SEQ ID NO: 34 - DNA, 20 bp, Mouse
cgctttggtgagaactagcc

SEQ ID NO: 35 - DNA, 20 bp, Mouse
catgtggagttgtgggagtg

SEQ ID NO: 36 - DNA, 20 bp, Mouse
aatgggcagaagacagatgg

SEQ ID NO: 37 - DNA, 20 bp, Mouse
tatcagggtctgtgaagccc

SEQ ID NO: 38 - DNA, 20 bp, Mouse
atacaggaccctttaccccg

SEQ ID NO: 39 - DNA, 20 bp, Mouse
cagtgtttctaggtccccca

SEQ ID NO: 40 - DNA, 20 bp, Mouse
gcctctgtctgccatctctc

SEQ ID NO: 41 - DNA, 20 bp, Mouse
ataatgttacctgcaggcgg

SEQ ID NO: 42 - DNA, 20 bp, Mouse
ctggaaacacccatgtcctc

SEQ ID NO: 43 - DNA, 20 bp, Mouse
cgggcacatggacactttta

SEQ ID NO: 44 - DNA, 20 bp, Mouse
gagcatgaagtgcaaggtga

SEQ ID NO: 45 - DNA, 20 bp, Mouse
cgtaggtggcacagttgaga

SEQ ID NO: 46 - DNA, 20 bp, Mouse
gctgttagtgaggtcagggc

SEQ ID NO: 47 - DNA, 20 bp, Mouse
cgtaggtggcacagttgaga

SEQ ID NO: 48 - DNA, 20 bp, Mouse
gagcatgaagtgcaaggtga

SEQ ID NO: 49 - DNA, 20 bp, Mouse
tcattttcctagcctcggtg

SEQ ID NO: 50 - DNA, 22 bp, Mouse
tctaagaagatgatgcagaccc

SEQ ID NO: 51 - DNA, 20 bp, Mouse
tgtccttcagggatagtgcc

SEQ ID NO: 52 - DNA, 20 bp, Mouse
ggcttcagcctcaagttctg

SEQ ID NO: 53 - DNA, 20 bp, Mouse
aaaacaaccaagttgccctg

SEQ ID NO: 54 - DNA, 20 bp, Mouse
ggcactgaaatgacctggat

SEQ ID NO: 55 - DNA, 20 bp, Mouse
aacaattcaagcaacctcgg

SEQ ID NO: 56 - DNA, 20 bp, Mouse
ctgttccttcccagactcca

SEQ ID NO: 57 - DNA, 20 bp, Mouse
ttcagtcacgcaaacctgag

SEQ ID NO: 58 - DNA, 20 bp, Mouse
gcccaggactttgtcactgt

SEQ ID NO: 59 - DNA, 20 bp, Mouse
ggtaacctgcagctccactc

SEQ ID NO: 60 - DNA, 20 bp, Mouse
gggacatgctcttggttcat

SEQ ID NO: 61 - DNA, 20 bp, Mouse
gaacaaagccgggtgattta

SEQ ID NO: 62 - DNA, 20 bp, Mouse
gccctcagttctcctagcct

SEQ ID NO: 63 - DNA, 20 bp, Mouse
ggcagagaagactggtggag

SEQ ID NO: 64 - DNA, 20 bp, Mouse
cccagacttagcgtctcagg

SEQ ID NO: 65 - DNA, 20 bp, Mouse
agcagagacctttggactcg

SEQ ID NO: 66 - DNA, 20 bp, Mouse
gaaggctgagtgagtcccag

SEQ ID NO: 67 - DNA, 20 bp, Mouse
ttgcacgaggagaaggtttt

SEQ ID NO: 68 - DNA, 20 bp, Mouse
gatgccaacgagacctgaat

SEQ ID NO: 69 - DNA, 20 bp, Mouse
agaagccaaaaccctcacct

SEQ ID NO: 70 - DNA, 20 bp, Mouse
aaaaagccctgcaagaactt

SEQ ID NO: 71 - DNA, 20 bp, Mouse
attcaggtctcgttggcatc

SEQ ID NO: 72 - DNA, 20 bp, Mouse
tgtccgcagtgtggaaacta

SEQ ID NO: 73 - DNA, 20 bp, Mouse
atgtccagggtagagagccc

SEQ ID NO: 74 - DNA, 20 bp, Mouse
ggagttctcctaccctggct

SEQ ID NO: 75 - DNA, 20 bp, Mouse
gaggctctgagcagtgtcaa

SEQ ID NO: 76 - DNA, 14 bp, Mouse
gcgatgttgttgcg

SEQ ID NO: 77 - DNA, 18 bp, Mouse
cagtgtctttccacattt

SEQ ID NO: 78 - DNA, 27 bp, Mouse
aggcatattgtataataaatttgtagt

SEQ ID NO: 79 - DNA, 19 bp, Mouse
ccggatgactctacttgac

SEQ ID NO: 80 - DNA, 20 bp, Mouse
gctgtttatggggtcgagaa

SEQ ID NO: 81 - DNA, 20 bp, Mouse
aatttctgaagcagggggat

SEQ ID NO: 82 - DNA, 20 bp, Mouse
tccccctgcttcagaaatta

SEQ ID NO: 83 - DNA, 20 bp, Mouse
agggggatgattgtgagtga

SEQ ID NO: 84 - DNA, 27 bp, Mouse
cttctttaatcaatctctgtctctgtg

SEQ ID NO: 85 - DNA, 20 bp, Mouse
gggcacatatgaacctcctg

SEQ ID NO: 86 - DNA, 20 bp, Mouse
ccaaactcttagcttcttca

SEQ ID NO: 87 - DNA, 21 bp, Mouse
acacagaagacactgaagaac

SEQ ID NO: 88 - DNA, 22 bp, Mouse
cagttgttagaagcaggatccc

SEQ ID NO: 89 - DNA, 23 bp, Mouse
aggtgcatatacctgggatactc

SEQ ID NO: 90 - DNA, 21 bp, Mouse
agagtttggtctcttcccctg

SEQ ID NO: 91 - DNA, 23 bp, Mouse
tatccaacacatttatgtctgcg

SEQ ID NO: 92 - DNA, 20 bp, Mouse
gccagtgtgctgaaagactg

SEQ ID NO: 93 - DNA, 20 bp, Mouse
agggacctggagacatcctt

SEQ ID NO: 94 - DNA, 23 bp, Mouse
ctgtaggctgcttttatcttttg

SEQ ID NO: 95 - DNA, 20 bp, Mouse
tgccccttcagcacatgcca

SEQ ID NO: 96 - DNA, 23 bp, Mouse
tgcagtgtgacatgtgcatagat

SEQ ID NO: 97 - DNA, 21 bp, Mouse
ggaaagccaggctacgcagaa

SEQ ID NO: 98 - DNA, 23 bp, Mouse
ctgtaggctgcttttatcttttg

SEQ ID NO: 99 - DNA, 20 bp, Mouse
tgccccttcagcacatgcca

SEQ ID NO: 100 - DNA, 22 bp, Mouse
tagtgtggttcctgactaacct

SEQ ID NO: 101 - DNA, 22 bp, Mouse
cggtctacatagtgagtgattc

SEQ ID NO: 102 - DNA, 22 bp, Mouse
aaaagcatcctgcatccttctg

SEQ ID NO: 103 - DNA, 22 bp, Mouse
gggttatacagagaaaccctgt

SEQ ID NO: 104 - DNA, 20 bp, Mouse
ttccaagctcacacatcagc

SEQ ID NO: 105 - DNA, 20 bp, Mouse
gtgctgctctgcattgagtg

SEQ ID NO: 106 - DNA, 20 bp, Mouse
gacagtgtgggagaatccgt

SEQ ID NO: 107 - DNA, 20 bp, Mouse
cccaaggcataggtcacaat

SEQ ID NO: 108 - DNA, 20 bp, Mouse
attgtgacctatgccttggg

SEQ ID NO: 109 - DNA, 20 bp, Mouse
cgaaggaccgtcatctgagt

SEQ ID NO: 110 - DNA, 20 bp, Mouse
ggctttgatgtgaaaaaggc

SEQ ID NO: 111 - DNA, 20 bp, Mouse
agctcctcatcgctcatgtt

SEQ ID NO: 112 - DNA, 20 bp, Mouse
tggaacatctctgtcggaag

SEQ ID NO: 113 - DNA, 20 bp, Mouse
ggctctcattgccaccttta

SEQ ID NO: 114 - DNA, 20 bp, Mouse
ccagagaacaggagacctgc

SEQ ID NO: 115 - DNA, 20 bp, Mouse
gtgctggatacactggcaga

SEQ ID NO: 116 - DNA, 20 bp, Mouse
gcgagacgagtgggtagttc

SEQ ID NO: 117 - DNA, 20 bp, Mouse
acactgaaacctcgcttgct

SEQ ID NO: 118 - DNA, 20 bp, Mouse
agcaagcgaggtttcagtgt

SEQ ID NO: 119 - DNA, 20 bp, Mouse
acggggcttgatccttttat

SEQ ID NO: 120 - DNA, 25 bp, Mouse
aagttcatgggcctcaccacctgtc

SEQ ID NO: 121 - DNA, 22 bp, Mouse
tactagctacccttcacatacc

SEQ ID NO: 122 - DNA, 21 bp, Mouse
acctagccactgtctcagtct

SEQ ID NO: 123 - DNA, 21 bp, Mouse
acagaagcagcatttacacag

SEQ ID NO: 124 - DNA, 20 bp, Mouse
tgggacagcttcctcaagat

SEQ ID NO: 125 - DNA, 20 bp, Mouse
aatgggaattgtgctcttgg

SEQ ID NO: 126 - DNA, 20 bp, Mouse
gggcatctggcaaagattta

SEQ ID NO: 127 - DNA, 20 bp, Mouse
agataacctgtgtgtcccgc

SEQ ID NO: 128 - DNA, 20 bp, Mouse
gatgtccgagaagggatgtg

SEQ ID NO: 129 - DNA, 20 bp, Mouse
tgtcagctttgagtgcatcc

SEQ ID NO: 130 - DNA, 20 bp, Mouse
acatgcaggctgtttgacct

SEQ ID NO: 131 - DNA, 20 bp, Mouse
tgtcagctttgagtgcatcc

SEQ ID NO: 132 - DNA, 20 bp, Mouse
gtgctctgcagacaaaccaa

SEQ ID NO: 133 - DNA, 20 bp, Mouse
gagccattttgacccttaaa

SEQ ID NO: 134 - DNA, 20 bp, Mouse
tttcagggtcaaaatggctc

SEQ ID NO: 135 - DNA, 17 bp, Mouse
tcgacagcaactgtgcg

SEQ ID NO: 136 - DNA, 20 bp, Mouse
ggtgagagtggggagatgaa

SEQ ID NO: 137 - DNA, 20 bp, Mouse
cccgggtgagtttaagaacc

SEQ ID NO: 138 - DNA, 20 bp, Mouse
ggtgagagtggggagatgaa

SEQ ID NO: 139 - DNA, 20 bp, Mouse
aggttaggcccaatttcctg

SEQ ID NO: 140 - DNA, 20 bp, Mouse
ccagggttgctgtactgaga

SEQ ID NO: 141 - DNA, 20 bp, Mouse
caggttaggcccaatttcct

SEQ ID NO: 142 - DNA, 20 bp, Mouse
ggtcagagtccttccttccc

SEQ ID NO: 143 - DNA, 20 bp, Mouse
tccaacttcacaggaaaccc

SEQ ID NO: 144 - DNA, 20 bp, Mouse
tttcctgtgaagttggaggg

SEQ ID NO: 145 - DNA, 20 bp, Mouse
cacccatatggcaaacatca

SEQ ID NO: 146 - DNA, 20 bp, Mouse
ggtcagagtccttccttccc

SEQ ID NO: 147 - DNA, 20 bp, Mouse
tccaacttcacaggaaaccc

SEQ ID NO: 148 - DNA, 20 bp, Mouse
tgatgtttgccatatgggtg

SEQ ID NO: 149 - DNA, 20 bp, Mouse
gcttgctgcttccgatatgt

SEQ ID NO: 150 - DNA, 19 bp, Mouse
ggaaaagggagtcgccata

SEQ ID NO: 151 - DNA, 20 bp, Mouse
gagccgcctaactctcacac

SEQ ID NO: 152 - DNA, 19 bp, Mouse
aggggataacctgcatagg

SEQ ID NO: 153 - DNA, 20 bp, Mouse
acaaaattgctcatttgccc

SEQ ID NO: 154 - DNA, 20 bp, Mouse
ccatccccactagccagata

SEQ ID NO: 155 - DNA, 20 bp, Mouse
gtcccctttgtcacagcaag

SEQ ID NO: 156 - DNA, 20 bp, Mouse
tgagcacaggatagctccac

SEQ ID NO: 157 - DNA, 20 bp, Mouse
aaaagaacacctgtttgggg

SEQ ID NO: 158 - DNA, 19 bp, Mouse
taaacctcggctgtgtgag

SEQ ID NO: 159 - DNA, 20 bp, Mouse
ccctcagtgacttcctgtga

SEQ ID NO: 160 - DNA, 20 bp, Mouse
caaaaccacatggttaccga

SEQ ID NO: 161 - DNA, 20 bp, Mouse
gccctattgccaaatgactt

SEQ ID NO: 162 - DNA, 20 bp, Mouse
ggcagaaaggaatcagaagc

SEQ ID NO: 163 - DNA, 20 bp, Mouse
cacattagccattgtcctgg

SEQ ID NO: 164 - DNA, 20 bp, Mouse
tcctttatgtccaacagcca

SEQ ID NO: 165 - DNA, 20 bp, Mouse
catggtctgtgatgtgacca

SEQ ID NO: 166 - DNA, 20 bp, Mouse
atacccttggtgagagcagg

SEQ ID NO: 167 - DNA, 20 bp, Mouse
gctgtcaaatgagaaaggca

SEQ ID NO: 168 - DNA, 20 bp, Mouse
tatttcatgctgggaccaaa

SEQ ID NO: 169 - DNA, 20 bp, Mouse
agagaaaaacagtgggggtg

SEQ ID NO: 170 - DNA, 20 bp, Mouse
cgggtcctctcttcaccata

SEQ ID NO: 171 - DNA, 20 bp, Mouse
ctacatttccctgagctgcc

SEQ ID NO: 172 - DNA, 20 bp, Mouse
gttgaccatgtcggtaaccc

SEQ ID NO: 173 - DNA, 20 bp, Mouse
ccacctcacggaaactgaat

SEQ ID NO: 174 - DNA, 20 bp, Mouse
ggtgtttggctcacaaacct

SEQ ID NO: 175 - DNA, 20 bp, Mouse
gatgcacacacaaaaatccg

SEQ ID NO: 176 - DNA, 20 bp, Mouse
atcacccaccagaacgaaaa

SEQ ID NO: 177 - DNA, 20 bp, Mouse
accctccaggagtaggtgct

SEQ ID NO: 178 - DNA, 20 bp, Mouse
gatgagacagtgggcaaggt

SEQ ID NO: 179 - DNA, 20 bp, Mouse
ttgtcaatagcaccaagcca

SEQ ID NO: 180 - DNA, 20 bp, Mouse
gccttaatagcccccttgtt

SEQ ID NO: 181 - DNA, 20 bp, Mouse
gcactcagcattgcacagat

SEQ ID NO: 182 - DNA, 20 bp, Mouse
ggacggacaattctggaaaa

SEQ ID NO: 183 - DNA, 20 bp, Mouse
ctatcacacctccgatgcct

SEQ ID NO: 184 - DNA, 20 bp, Mouse
caagctggtagaatccccaa

SEQ ID NO: 185 - DNA, 20 bp, Mouse
tctttggagaagcagaccgt

SEQ ID NO: 186 - DNA, 20 bp, Mouse
tacagcatatgcatgccagg

SEQ ID NO: 187 - DNA, 20 bp, Mouse
attcctcagggcattacacg

SEQ ID NO: 188 - DNA, 20 bp, Mouse
gcaatctcttgtgtccaggc

SEQ ID NO: 189 - DNA, 20 bp, Mouse
attcctcagggcattacacg

SEQ ID NO: 190 - DNA, 20 bp, Mouse
tacagcatatgcatgccagg

SEQ ID NO: 191 - DNA, 20 bp, Mouse
ggcctggacacaagagattg

SEQ ID NO: 192 - DNA, 20 bp, Mouse
aagtgggtggacagtgaagg

SEQ ID NO: 193 - DNA, 20 bp, Mouse
cagcttcctccatcttctgg

SEQ ID NO: 194 - DNA, 20 bp, Mouse
agagcctccagtagatggca

SEQ ID NO: 195 - DNA, 20 bp, Mouse
tcgtggacaagctccttctt

SEQ ID NO: 196 - DNA, 20 bp, Mouse
catcgagtatgtcaatggcg

SEQ ID NO: 197 - DNA, 20 bp, Mouse
ttgtccagttttaggtcccg

SEQ ID NO: 198 - DNA, 20 bp, Mouse
cagactgggttttccgacat

SEQ ID NO: 199 - DNA, 20 bp, Mouse
gtcaaagttgtccaggccat

SEQ ID NO: 200 - DNA, 18 bp, Mouse
aggacggaccccaagatg

SEQ ID NO: 201 - DNA, 20 bp, Mouse
tgtctcgcacttcctcacag

SEQ ID NO: 202 - DNA, 20 bp, Mouse
ccagaagatggaggaagctg

SEQ ID NO: 203 - DNA, 20 bp, Mouse
tctactggaggctcttggga

SEQ ID NO: 204 - DNA, 20 bp, Mouse
gaaaaacgaccagatttacg

SEQ ID NO: 205 - DNA, 20 bp, Mouse
gatctcagcagcatagaacc

SEQ ID NO: 206 - DNA, 20 bp, Mouse
acacattaagctgacggact

SEQ ID NO: 207 - DNA, 20 bp, Mouse
caaacataaggacacccagt

SEQ ID NO: 208 - DNA, 20 bp, Mouse
actgggtgtccttatgtttg

SEQ ID NO: 209 - DNA, 20 bp, Mouse
cctctctttgggatccttat

SEQ ID NO: 210 - DNA, 20 bp, Mouse
gtcataaagaggatcgacca

SEQ ID NO: 211 - DNA, 20 bp, Mouse
gctctgtctagaagtgcctg

SEQ ID NO: 212 - DNA, 18 bp, Mouse
accaagaccgaagagggg

SEQ ID NO: 213 - DNA, 22 bp, Mouse
ggcattacacgctaacttttcc

SEQ ID NO: 214 - DNA, 20 bp, Mouse
agtgccaccaacctggtaag

SEQ ID NO: 215 - DNA, 18 bp, Mouse
aagtgcctgcagggatgc

SEQ ID NO: 216 - DNA, 20 bp, Mouse
tgctttggtgagcaatgttt

SEQ ID NO: 217 - DNA, 20 bp, Mouse
agggacacccttaccaggtt

SEQ ID NO: 218 - DNA, 20 bp, Mouse
ctgatgctttggtgagcaat

SEQ ID NO: 219 - DNA, 19 bp, Mouse
gggacacccttaccaggtt

SEQ ID NO: 220 - DNA, 20 bp, Mouse
acaggacaaatgctgggttg

SEQ ID NO: 221 - DNA, 20 bp, Mouse
gtggtaaagaacgcttggct

SEQ ID NO: 222 - DNA, 24 bp, Mouse
ggtatctcacttggtaggaacctc

SEQ ID NO: 223 - DNA, 17 bp, Mouse
aagaacgcttggctggc

SEQ ID NO: 224 - DNA, 20 bp, Mouse
gccgatcctggtgatgtact

SEQ ID NO: 225 - DNA, 20 bp, Mouse
acaatggctcaaaaccgttc

SEQ ID NO: 226 - DNA, 20 bp, Mouse
gccttgggaatttaccacct

SEQ ID NO: 227 - DNA, 20 bp, Mouse
agtacatcaccaggatcggc

SEQ ID NO: 228 - DNA, 20 bp, Mouse
taaaaggccatgcgataagc

SEQ ID NO: 229 - DNA, 20 bp, Mouse
agagctctgtggggttctca

SEQ ID NO: 230 - DNA, 20 bp, Mouse
gaaggggacagtgttggaga

SEQ ID NO: 231 - DNA, 20 bp, Mouse
tccatcaaggaaggatccac

SEQ ID NO: 232 - DNA, 19 bp, Mouse
ggtgggtaatgattggact

SEQ ID NO: 233 - DNA, 19 bp, Mouse
tgacgtggagggaactgcc

SEQ ID NO: 234 - DNA, 20 bp, Mouse
tgagatctggtgccctctct

SEQ ID NO: 235 - DNA, 20 bp, Mouse
gcctgatctaggctggaaaa

SEQ ID NO: 236 - DNA, 20 bp, Mouse
aggcagaaagcagacaagga

SEQ ID NO: 237 - DNA, 20 bp, Mouse
cgacagcacttgtgaccact

SEQ ID NO: 238 - DNA, 20 bp, Mouse
ctgcagatgtagaccaggca

SEQ ID NO: 239 - DNA, 20 bp, Mouse
ctgtggtggattggacagtg

SEQ ID NO: 240 - DNA, 20 bp, Mouse
ttgcctaacactcccaaacc

SEQ ID NO: 241 - DNA, 20 bp, Mouse
tattaggagcaccaccaggc

SEQ ID NO: 242 - DNA, 20 bp, Mouse
acctgtcttgtgggtggaag

SEQ ID NO: 243 - DNA, 20 bp, Mouse
ctgtggtggattggacagtg

SEQ ID NO: 244 - DNA, 20 bp, Mouse
gtggcttggtgctattgaca

SEQ ID NO: 245 - DNA, 20 bp, Mouse
ggggctattaaggccatttt

SEQ ID NO: 246 - DNA, 21 bp, Mouse
caattgaggaatggctaccaa

SEQ ID NO: 247 - DNA, 20 bp, Mouse
tggcttcatgtccattgtgt

SEQ ID NO: 248 - DNA, 22 bp, Mouse
cagaaccacaaaggtaaattgc

SEQ ID NO: 249 - DNA, 21 bp, Mouse
tcatgtttgctgtccagtttg

SEQ ID NO: 250 - DNA, 29 bp, Homo sapiens
gccaccatgctgggccctgctgtcctggg

SEQ ID NO: 251 - DNA, 24 bp, Homo sapiens
tcactcatgtttcccctgatttcc

SEQ ID NO: 252 - DNA, 20 bp, Homo sapiens
ctgatttcctgtgttcccgt

SEQ ID NO: 253 - DNA, 20 bp, Homo sapiens
catgctggcctacttcatca

SEQ ID NO: 254 - DNA, 29 bp, Homo sapiens
gccttgcaggtcagctacggtgctagcat

SEQ ID NO: 255 - DNA, 24 bp, Homo sapiens
tcactcatgtttcccctgatttcc

SEQ ID NO: 256 - DNA, 20 bp, Homo sapiens
aggaagcagagaaaggccag

SEQ ID NO: 257 - DNA, 20 bp, Homo sapiens
tcagaactgcctctgagctg

SEQ ID NO: 258 - DNA, 20 bp, Homo sapiens
tcttcacgtactgggggaac

SEQ ID NO: 259 - DNA, 20 bp, Homo sapiens
actacagcatcagcagcagg

SEQ ID NO: 260 - DNA, 20 bp, Homo sapiens
aagctgaagaacttcccggt

SEQ ID NO: 261 - DNA, 20 bp, Homo sapiens
tgggctacgacctctttgat

SEQ ID NO: 262 - DNA, 20 bp, Homo sapiens
atcttcaggcgctctgtcct

SEQ ID NO: 263 - DNA, 20 bp, Homo sapiens
gtacgacctgaagctgtggg

SEQ ID NO: 264 - DNA, 19 bp, Homo sapiens
atcttcaggcgctctgtcc

SEQ ID NO: 265 - DNA, 20 bp, Homo sapiens
gtacgacctgaagctgtggg

SEQ ID NO: 266 - DNA, 19 bp, Homo sapiens
atcttcaggcgctctgtcc

SEQ ID NO: 267 - DNA, 21 bp, Homo sapiens
gagtacgacctgaagctgtgg

SEQ ID NO: 268 - DNA, 19 bp, Homo sapiens
atcttcaggcgctctgtcc

SEQ ID NO: 269 - DNA, 19 bp, Homo sapiens
tacgacctgaagctgtggg

SEQ ID NO: 270 - DNA, 19 bp, Homo sapiens
atcttcaggcgctctgtcc

SEQ ID NO: 271 - DNA, 19 bp, Homo sapiens
tacgacctgaagctgtggg

SEQ ID NO: 272 - DNA, 18 bp, Homo sapiens
gctgtcccgatggtgaac

SEQ ID NO: 273 - DNA, 19 bp, Homo sapiens
accttttgtggccaggatg

SEQ ID NO: 274 - DNA, 18 bp, Homo sapiens
gctgtcccgatggtgaac

SEQ ID NO: 275 - DNA, 19 bp, Homo sapiens
caccttttgtggccaggat

SEQ ID NO: 276 - DNA, 18 bp, Homo sapiens
gctgtcccgatggtgaac

SEQ ID NO: 277 - DNA, 18 bp, Homo sapiens
ccttttgtggccaggatg

SEQ ID NO: 278 - DNA, 18 bp, Homo sapiens
cctgaaccagtgggctgt

SEQ ID NO: 279 - DNA, 19 bp, Homo sapiens
accttttgtggccaggatg

SEQ ID NO: 280 - DNA, 18 bp, Homo sapiens
cctgaaccagtgggctgt

SEQ ID NO: 281 - DNA, 19 bp, Homo sapiens
caccttttgtggccaggat

SEQ ID NO: 282 - DNA, 20 bp, Homo sapiens
tcatgtttcccctgatttcc

SEQ ID NO: 283 - DNA, 20 bp, Homo sapiens
catgctggcctacttcatca

SEQ ID NO: 284 - DNA, 20 bp, Homo sapiens
atgagcaggtaacacctggg

SEQ ID NO: 285 - DNA, 20 bp, Homo sapiens
tcatcacctgggtctccttt

SEQ ID NO: 286 - DNA, 20 bp, Homo sapiens
atgagcaggtaacacctggg

SEQ ID NO: 287 - DNA, 20 bp, Homo sapiens
ttcatcacctgggtctcctt

SEQ ID NO: 288 - DNA, 20 bp, Mouse
tgggttgtgttctctggttg

SEQ ID NO: 289 - DNA, 21 bp, Mouse
cctttttacagtctgccaggt

SEQ ID NO: 290 - DNA, 20 bp, Mouse
tgggttgtgttctctggttg

SEQ ID NO: 291 - DNA, 21 bp, Mouse
gatcccctttttacagtctgc

SEQ ID NO: 292 - DNA, 20 bp, Mouse
acggggttggtactgtgtgt

SEQ ID NO: 293 - DNA, 20 bp, Mouse
cacccattgttagtgctgga

SEQ ID NO: 294 - DNA, 20 bp, Mouse
acggggttggtactgtgtgt

SEQ ID NO: 295 - DNA, 20 bp, Mouse
cacacacccacccattgtta

SEQ ID NO: 296 - DNA, 20 bp, Mouse
tgcattggccagactagaaa

SEQ ID NO: 297 - DNA, 19 bp, Mouse
cggctgggctatgacctat

SEQ ID NO: 298 - DNA, 20 bp, Mouse
tgcattggccagactagaaa

SEQ ID NO: 299 - DNA, 20 bp, Mouse
cggctgggctatgacctatt

SEQ ID NO: 300 - DNA, 20 bp, Mouse
gttctgcagcatgatgtcgt

SEQ ID NO: 301 - DNA, 20 bp, Mouse
ggcagttgtgactctgttgc

SEQ ID NO: 302 - DNA, 20 bp, Mouse
gttctgcagcatgatgtcgt

SEQ ID NO: 303 - DNA, 20 bp, Mouse
ctgcaggcagttgtgactct

SEQ ID NO: 304 - DNA, 20 bp, Mouse
ccatcctttttgcctgtctt

SEQ ID NO: 305 - DNA, 20 bp, Mouse
tctggaggaacatgtgatgg

SEQ ID NO: 306 - DNA, 20 bp, Mouse
caccatcctttttgcctgtc

SEQ ID NO: 307 - DNA, 19 bp, Mouse
gaacatgtgatggggcaac

SEQ ID NO: 308 - DNA, 19 bp, Mouse
caaagcagcaggaggagtg

SEQ ID NO: 309 - DNA, 20 bp, Mouse
aaatgtactggccaggcaac

SEQ ID NO: 310 - DNA, 20 bp, Mouse
agtgctagacccagcaccag

SEQ ID NO: 311 - DNA, 20 bp, Mouse
aaatgtactggccaggcaac

SEQ ID NO: 312 - DNA, 20 bp, Mouse
gcactgaccagtctgtcacc

SEQ ID NO: 313 - DNA, 20 bp, Mouse
gtccccagagaaaagcacag

SEQ ID NO: 314 - DNA, 20 bp, Mouse
cagtctgtcaccacctctgg

SEQ ID NO: 315 - DNA, 20 bp, Mouse
cagtggtccccagagaaaag

SEQ ID NO: 316 - DNA, 20 bp, Mouse
tactattcggggcttgttgg

SEQ ID NO: 317 - DNA, 20 bp, Mouse
gcagcactatgtgcctggta

SEQ ID NO: 318 - DNA, 20 bp, Mouse
tactattcggggcttgttgg

SEQ ID NO: 319 - DNA, 20 bp, Mouse
gcctggtatttgatcgcttt

SEQ ID NO: 320 - DNA, 20 bp, Mouse
gctcagctagggatggagaa

SEQ ID NO: 321 - DNA, 20 bp, Mouse
cagctcagggacacaatgaa

SEQ ID NO: 322 - DNA, 20 bp, Mouse
tcctacaggctagggctcag

SEQ ID NO: 323 - DNA, 20 bp, Mouse
cagctcagggacacaatgaa

SEQ ID NO: 324 - DNA, 20 bp, Mouse
gggactgatgtgtggcttgt

SEQ ID NO: 325 - DNA, 20 bp, Mouse
aggcgtcccaggaatagaag

SEQ ID NO: 326 - DNA, 21 bp, Mouse
ggactgatgtgtggcttgttt

SEQ ID NO: 327 - DNA, 20 bp, Mouse
aggcgtcccaggaatagaag

SEQ ID NO: 328 - DNA, 20 bp, Mouse
tgtttctgttctggtggctg

SEQ ID NO: 329 - DNA, 20 bp, Mouse
atctgcaggcaggatcagac

SEQ ID NO: 330 - DNA, 20 bp, Mouse
ctcagtggtgggtgacagtg

SEQ ID NO: 331 - DNA, 20 bp, Mouse
atctgcaggcaggatcagac

SEQ ID NO: 332 - DNA, 20 bp, Mouse
acacacagtaccaaccccgt

SEQ ID NO: 333 - DNA, 20 bp, Mouse
cctgtggtgatcaagaagca

SEQ ID NO: 334 - DNA, 20 bp, Mouse
tgcttcttgatcaccacagg

SEQ ID NO: 335 - DNA, 20 bp, Mouse
gcaacagagtcacaactgcc

SEQ ID NO: 336 - DNA, 20 bp, Mouse
acacacagtaccaaccccgt

SEQ ID NO: 337 - DNA, 20 bp, Mouse
gcaacagagtcacaactgcc

SEQ ID NO: 338 - DNA, 20 bp, Mouse
gggtttatgtggcaagcact

SEQ ID NO: 339 - DNA, 20 bp, Mouse
actccatttgccttttgtgg

SEQ ID NO: 340 - DNA, 20 bp, Mouse
cgctacttcgcttttatccg

SEQ ID NO: 341 - DNA, 20 bp, Mouse
atgatgacgtacgacgacga

SEQ ID NO: 342 - DNA, 21 bp, Mouse
gaaaacaatcggggagaagtc

SEQ ID NO: 343 - DNA, 20 bp, Mouse
tgaaattatcacacgccagg

SEQ ID NO: 344 - DNA, 20 bp, Mouse
agtgagaggcccagtctcaa

SEQ ID NO: 345 - DNA, 20 bp, Mouse
gatctgatgccctcttctgc

SEQ ID NO: 346 - DNA, 20 bp, Mouse
gctagccttgaagccaacac

SEQ ID NO: 347 - DNA, 20 bp, Mouse
tgaacagcatgcttacccag

SEQ ID NO: 348 - DNA, 20 bp, Mouse
tccctagaggcctgtctgtc

SEQ ID NO: 349 - DNA, 20 bp, Mouse
tcgtctcggagcctcttcta

SEQ ID NO: 350 - DNA, 20 bp, Mouse
gatagtcccttagccagccc

SEQ ID NO: 351 - DNA, 20 bp, Mouse
gccatagctcctcactgctc

SEQ ID NO: 352 - DNA, 20 bp, Mouse
cagagtgggctctggtcttc

SEQ ID NO: 353 - DNA, 20 bp, Mouse
ttgtgttcagatgctcctgc

SEQ ID NO: 354 - DNA, 20 bp, Mouse
ttatttctgtgctagccgcc

SEQ ID NO: 355 - DNA, 20 bp, Mouse
atcaagtcaacgtccccaag

SEQ ID NO: 356 - DNA, 20 bp, Mouse
acctggcctgtgctaatctc

SEQ ID NO: 357 - DNA, 20 bp, Mouse
gcaccaaccctaagaaagca

SEQ ID NO: 358 - DNA, 22 bp, Mouse
tcaggctaacctcaaactcaca

SEQ ID NO: 359 - DNA, 27 bp, Mouse
aaagaaaagaaaagaaaaagtcagaca

SEQ ID NO: 360 - DNA, 20 bp, Mouse
cccagaactccatcctcaaa

SEQ ID NO: 361 - DNA, 20 bp, Mouse
cccaacctgtggtcagctat

SEQ ID NO: 362 - DNA, 20 bp, Mouse
ggggcaggtgggtaataagt

SEQ ID NO: 363 - DNA, 20 bp, Mouse
caaaagcccaactccttgag

SEQ ID NO: 364 - DNA, 20 bp, Mouse
gctcagtgggtaagagcacc

SEQ ID NO: 365 - DNA, 20 bp, Mouse
ctaccctgccgctaatctca

SEQ ID NO: 366 - DNA, 20 bp, Mouse
cagttagcaccccaccctaa

SEQ ID NO: 367 - DNA, 20 bp, Mouse
tctgcacctctgttcacctg

SEQ ID NO: 368 - DNA, 20 bp, Mouse
acctctagggtttacgggga

SEQ ID NO: 369 - DNA, 20 bp, Mouse
cctcaggtagtgcaagctcc

SEQ ID NO: 370 - DNA, 20 bp, Mouse
tcagttaccaagggtttcgg

SEQ ID NO: 371 - DNA, 20 bp, Mouse
ataggttgtcacaggccagg

SEQ ID NO: 372 - DNA, 20 bp, Mouse
tcagttaccaagggtttcgg

SEQ ID NO: 373 - DNA, 20 bp, Mouse
ataggttgtcacaggccagg

SEQ ID NO: 374 - DNA, 20 bp, Mouse
gtggttgctgggatttgaac

SEQ ID NO: 375 - DNA, 20 bp, Mouse
caagcaaccaaacaaccaaa

SEQ ID NO: 376 - DNA, 20 bp, Mouse
tccggaggaccataaatctg

SEQ ID NO: 377 - DNA, 20 bp, Mouse
cacagtcccagtcattccct

SEQ ID NO: 378 - DNA, 20 bp, Mouse
gtcccaaaagctagcacagg

SEQ ID NO: 379 - DNA, 20 bp, Mouse
tcatgagccaccatgtgatt

SEQ ID NO: 380 - DNA, 20 bp, Mouse
gaccttcggaagagcagttg

SEQ ID NO: 381 - DNA, 20 bp, Mouse
agtgtgtgtcgccatatcca

SEQ ID NO: 382 - DNA, 20 bp, Mouse
cctactctctctccccgctt

SEQ ID NO: 383 - DNA, 20 bp, Mouse
ggaaaatgtttggccttgaa

SEQ ID NO: 384 - DNA, 20 bp, Mouse
ctggagtgaaaggcaggaag

SEQ ID NO: 385 - DNA, 20 bp, Mouse
aggcggcaccatatgaataa

SEQ ID NO: 386 - DNA, 21 bp, Mouse
tgagagtgggaattctgttca

SEQ ID NO: 387 - DNA, 20 bp, Mouse
ggatgtaattggtggcaagg

SEQ ID NO: 388 - DNA, 20 bp, Mouse
ctgttggaggaggtggccta

SEQ ID NO: 389 - DNA, 21 bp, Mouse
tgcttgtatgtttttcctcgt

SEQ ID NO: 390 - DNA, 20 bp, Mouse
tgagagtgccctcctctttg

SEQ ID NO: 391 - DNA, 18 bp, Mouse
gaacccctgaccccagac

SEQ ID NO: 392 - DNA, 22 bp, Mouse
tgaagtgcagatttttacatgg

SEQ ID NO: 393 - DNA, 20 bp, Mouse
gttttggggtggaaaaggat

SEQ ID NO: 394 - DNA, 20 bp, Mouse
ccgtcgacatttaggtgaca

SEQ ID NO: 395 - DNA, 20 bp, Mouse
gatactggggtggtgggtaa

SEQ ID NO: 396 - DNA, 20 bp, Mouse
ccgtcgacatttaggtgaca

SEQ ID NO: 397 - DNA, 20 bp, Mouse
cgtcccagctgtgtaactga

SEQ ID NO: 398 - DNA, 21 bp, Mouse
ggaagcaaatgctccactaaa

SEQ ID NO: 399 - DNA, 20 bp, Mouse
tatccctagccccttgtgtg

SEQ ID NO: 400 - DNA, 20 bp, Mouse
ccgtcgacatttaggtgaca

SEQ ID NO: 401 - DNA, 20 bp, Mouse
gggtcctgttggtagtgacc

SEQ ID NO: 402 - DNA, 20 bp, Mouse
tataagcagcccctcattgg

SEQ ID NO: 403 - DNA, 20 bp, Mouse
caggccagacactgcttaca

SEQ ID NO: 404 - DNA, 20 bp, Mouse
ccttgggatctggtgtgact

SEQ ID NO: 405 - DNA, 20 bp, Mouse
tgggtttagagtacggctgg

SEQ ID NO: 406 - DNA, 20 bp, Mouse
acccatttcctaatcccctg

SEQ ID NO: 407 - DNA, 20 bp, Mouse
atctctccagcccctctcag

SEQ ID NO: 408 - DNA, 20 bp, Mouse
gggctgggaattgaacctat

SEQ ID NO: 409 - DNA, 20 bp, Mouse
tgaatcccttacagccttgc

SEQ ID NO: 410 - DNA, 20 bp, Mouse
gccccataaaatccactcct

SEQ ID NO: 411 - DNA, 20 bp, Mouse
gctccggaaggctagaagat

SEQ ID NO: 412 - DNA, 20 bp, Mouse
ggtttgggagtgttaggcaa

SEQ ID NO: 413 - DNA, 20 bp, Mouse
actcagttggcctctcctca

SEQ ID NO: 414 - DNA, 19 bp, Mouse
acagaaatccctcatgcga

SEQ ID NO: 415 - DNA, 21 bp, Mouse
tcagtgtggaccagaaagtcc

SEQ ID NO: 416 - DNA, 22 bp, Mouse
tctgcaagtcagctcttgataa

SEQ ID NO: 417 - DNA, 23 bp, Mouse
actcataagggtcaagctgtctg

SEQ ID NO: 418 - DNA, 20 bp, Mouse
tctccccttttaccactccc

SEQ ID NO: 419 - DNA, 20 bp, Mouse
gcaaggagtcaaaaacagca

SEQ ID NO: 420 - DNA, 20 bp, Mouse
gctagttggggaacaaacca

SEQ ID NO: 421 - DNA, 20 bp, Mouse
actgcaaatgtccaactcca

SEQ ID NO: 422 - DNA, 20 bp, Mouse
cagttacacagctgggacga

SEQ ID NO: 423 - DNA, 20 bp, Mouse
gcaagagcctagcaatccac

SEQ ID NO: 424 - DNA, 20 bp, Mouse
cagtttagcaccccacccta

SEQ ID NO: 425 - DNA, 20 bp, Mouse
tctgcacctctgttcacctg

SEQ ID NO: 426 - DNA, 20 bp, Mouse
gggttccacttgatgctgat

SEQ ID NO: 427 - DNA, 20 bp, Mouse
tggtctgtttcctggagctt

SEQ ID NO: 428 - DNA, 21 bp, Mouse
tgtagggaatgtttctgcacc

SEQ ID NO: 429 - DNA, 20 bp, Mouse
acatggaacaggattctggc

SEQ ID NO: 430 - DNA, 20 bp, Mouse
gcaggcaaacagacagacaa

SEQ ID NO: 431 - DNA, 20 bp, Mouse
atgggggatcccttactgac

SEQ ID NO: 432 - DNA, 20 bp, Mouse
cggtcaggagtagtgtgggt

SEQ ID NO: 433 - DNA, 20 bp, Mouse
cagcagctgatattgaggca

SEQ ID NO: 434 - DNA, 22 bp, Mouse
aatgatgaagtgtcagcctcag

SEQ ID NO: 435 - DNA, 20 bp, Mouse
caacagaactcaaagcctgg

SEQ ID NO: 436 - DNA, 20 bp, Mouse
agcaggcacaggtctcttgt

SEQ ID NO: 437 - DNA, 20 bp, Mouse
aagaacaggacagtggtggg

SEQ ID NO: 438 - DNA, 20 bp, Mouse
cagcgattggctcttctctt

SEQ ID NO: 439 - DNA, 20 bp, Mouse
ggggcttcctttctgaggta

SEQ ID NO: 440 - DNA, 20 bp, Mouse
agctcaggtccagcttggta

SEQ ID NO: 441 - DNA, 20 bp, Mouse
attttcccctcctgcttctc

SEQ ID NO: 442 - DNA, 20 bp, Mouse
ccaagcctctgctggttatc

SEQ ID NO: 443 - DNA, 20 bp, Mouse
tgagggtggagaatggaaag

SEQ ID NO: 444 - DNA, 20 bp, Mouse
gccccataaaatccactcct

SEQ ID NO: 445 - DNA, 20 bp, Mouse
ttgcctaacactcccaaacc

SEQ ID NO: 446 - DNA, 20 bp, Mouse
cagttacacagctgggacga

SEQ ID NO: 447 - DNA, 20 bp, Mouse
gcaagagcctagcaatccac

SEQ ID NO: 448 - DNA, 20 bp, Mouse
cagcaccttcctctggtctc

SEQ ID NO: 449 - DNA, 20 bp, Mouse
tgtctccagaggttctgcct

SEQ ID NO: 450 - DNA, 24 bp, Mouse
tggtggtgtaatactattcctttg

SEQ ID NO: 451 - DNA, 26 bp, Mouse
tctttaatttttggctttttgataca

SEQ ID NO: 452 - DNA, 20 bp, Mouse
cagctgtgtgcatgttgacc

SEQ ID NO: 453 - DNA, 20 bp, Mouse
catcatgaagactcagggca

SEQ ID NO: 454 - DNA, 20 bp, Mouse
gtccacacctggcttttgtt

SEQ ID NO: 455 - DNA, 20 bp, Mouse
cagcactcagtgaggttcca

SEQ ID NO: 456 - DNA, 20 bp, Mouse
atgtaatggaagggctgctg

SEQ ID NO: 457 - DNA, 20 bp, Mouse
cagcactcagtgaggttcca

SEQ ID NO: 458 - DNA, 21 bp, Mouse
aaacaggcatgaaactcagga

SEQ ID NO: 459 - DNA, 20 bp, Mouse
gggtatcattgtcacctcca

SEQ ID NO: 460 - DNA, 20 bp, Mouse
cacaggccaagttgttgttg

SEQ ID NO: 461 - DNA, 20 bp, Mouse
caggggaccttctgaatgat

SEQ ID NO: 462 - DNA, 20 bp, Mouse
agctcaggtccagcttggta

SEQ ID NO: 463 - DNA, 20 bp, Mouse
accacaaaattttcccctcc

SEQ ID NO: 464 - DNA, 20 bp, Mouse
cgggacctaaaactggacaa

SEQ ID NO: 465 - DNA, 20 bp, Mouse
tggggacagttaccaggaag

SEQ ID NO: 466 - DNA, 20 bp, Mouse
ccggaggaccataaatctga

SEQ ID NO: 467 - DNA, 20 bp, Mouse
cctcaaaaacaagcctgagc

SEQ ID NO: 468 - DNA, 22 bp, Mouse
ccttcagaaatgtgtttggaca

SEQ ID NO: 469 - DNA, 20 bp, Mouse
tcctgagttcaaatcccagc

SEQ ID NO: 470 - DNA, 20 bp, Mouse
ctttccattctccaccctca

SEQ ID NO: 471 - DNA, 20 bp, Mouse
aggtcctagggagaggtcca

SEQ ID NO: 472 - DNA, 20 bp, Mouse
aggcctacccaaggacatct

SEQ ID NO: 473 - DNA, 20 bp, Mouse
gcagtgagctgcagagtttg

SEQ ID NO: 474 - DNA, 20 bp, Mouse
agacaccctaggtcctgctg

SEQ ID NO: 475 - DNA, 22 bp, Mouse
tgatctttccaaacgcataaga

SEQ ID NO: 476 - DNA, 20 bp, Mouse
gcaagcaacctgaacatgaa

SEQ ID NO: 477 - DNA, 20 bp, Mouse
gcttacgatggtcgtgaggt

SEQ ID NO: 478 - DNA, 20 bp, Mouse
acatgcctgcctatctttgc

SEQ ID NO: 479 - DNA, 20 bp, Mouse
ggaacctgttttccatggtg

SEQ ID NO: 480 - DNA, 20 bp, Mouse
accttgttcctggtgtgagc

SEQ ID NO: 481 - DNA, 20 bp, Mouse
tagctgggacgtggtatggt

SEQ ID NO: 482 - DNA, 20 bp, Mouse
ccatgggagaccagaaggta

SEQ ID NO: 483 - DNA, 20 bp, Mouse
tgagtgtcctctgcctgatg

SEQ ID NO: 484 - DNA, 20 bp, Mouse
gcgctgacatcctcctatgt

SEQ ID NO: 485 - DNA, 20 bp, Mouse
cccactatggtcccagagaa

SEQ ID NO: 486 - DNA, 20 bp, Mouse
ttgcacgtctttgtttcgag

SEQ ID NO: 487 - DNA, 24 bp, Mouse
aaaggggaatagacctgagtagaa

SEQ ID NO: 488 - DNA, 20 bp, Mouse
ccaagagtcagccttggagt

SEQ ID NO: 489 - DNA, 20 bp, Mouse
ggacaggtagctcacccaac

SEQ ID NO: 490 - DNA, 19 bp, Mouse
tgccagctttggctatcat

SEQ ID NO: 491 - DNA, 20 bp, Mouse
ttcattgtgtccctgagctg

SEQ ID NO: 492 - DNA, 24 bp, Mouse
agctttggctatcatgggtctcag

SEQ ID NO: 493 - DNA, 22 bp, Mouse
accaccgccactgttctcatct

SEQ ID NO: 494 - DNA, 20 bp, Mouse
tgtgggggaagaacatagaa

SEQ ID NO: 495 - DNA, 22 bp, Mouse
tgatgtgtggcttgtttctctt

SEQ ID NO: 496 - DNA, 20 bp, Mouse
ataggtggggagggagctaa

SEQ ID NO: 497 - DNA, 22 bp, Mouse
tgatgtgtggcttgtttctctt

SEQ ID NO: 498 - DNA, 20 bp, Homo sapiens
tgtgcctgtcacagcaactt

SEQ ID NO: 499 - DNA, 20 bp, Homo sapiens
catgctagcaccgtagctga

SEQ ID NO: 500 - DNA, 20 bp, Homo sapiens
ggagaccttcccctccttct

SEQ ID NO: 501 - DNA, 20 bp, Homo sapiens
gctgtagttgaagagggcgt

SEQ ID NO: 502 - DNA, 18 bp, Homo sapiens
gtgcttggcttcctccag

SEQ ID NO: 503 - DNA, 20 bp, Homo sapiens
caggtcgtactccatgtcca

SEQ ID NO: 504 - DNA, 20 bp, Homo sapiens
tggagtacgacctgaagctg

SEQ ID NO: 505 - DNA, 20 bp, Homo sapiens
actcatcctggccacaaaag

SEQ ID NO: 506 - DNA, 19 bp, Homo sapiens
gaacaggaggacgctgagg

SEQ ID NO: 507 - DNA, 20 bp, Homo sapiens
cttttgtggccaggatgagt

SEQ ID NO: 508 - DNA, 20 bp, Homo sapiens
tcacctcacctggttgtcag

SEQ ID NO: 509 - DNA, 20 bp, Homo sapiens
gtacgacctgaagctgtggg

SEQ ID NO: 510 - DNA, 27 bp, Homo sapiens
ggctgagatcacagggttgggtcactc

SEQ ID NO: 511 - DNA, 27 bp, Homo sapiens
ccgtgcctgttggaagttgcctctgcc

SEQ ID NO: 512 - DNA, 20 bp, Mouse
aattcccagcaaccactcac

SEQ ID NO: 513 - DNA, 20 bp, Mouse
cagacactccagaagagggc

SEQ ID NO: 514 - DNA, 20 bp, Mouse
tgactgctcttccgaaggtt

SEQ ID NO: 515 - DNA, 20 bp, Mouse
tttgtggaatagccaaagcc

SEQ ID NO: 516 - DNA, 20 bp, Mouse
tctctcctctcttctccccc

SEQ ID NO: 517 - DNA, 20 bp, Mouse
agcagggtgcatcaccttat

SEQ ID NO: 518 - DNA, 20 bp, Mouse
taggagtgccccataggttg

SEQ ID NO: 519 - DNA, 20 bp, Mouse
tcattgtacccagccagtca

SEQ ID NO: 520 - DNA, 20 bp, Mouse
aggactgagcctggatgaga

SEQ ID NO: 521 - DNA, 20 bp, Mouse
ctgggcgttttgttttgttt

SEQ ID NO: 522 - DNA, 20 bp, Mouse
cttcctcctgcagctaccac

SEQ ID NO: 523 - DNA, 20 bp, Mouse
accctgctacaacgcagact

SEQ ID NO: 524 - DNA, 20 bp, Mouse
tccaaccttgacacccattt

SEQ ID NO: 525 - DNA, 20 bp, Mouse
agccagggctacacagagaa

SEQ ID NO: 526 - DNA, 20 bp, Mouse
ctgcttttcctcagcaactg

SEQ ID NO: 527 - DNA, 20 bp, Mouse
attcgccgttagaagctagg

SEQ ID NO: 528 - DNA, 20 bp, Mouse
aactgtacgtggctgctggt

SEQ ID NO: 529 - DNA, 20 bp, Mouse
attcgccgttagaagctagg

SEQ ID NO: 530 - DNA, 20 bp, Mouse
gccaggtgacccttatgaaa

SEQ ID NO: 531 - DNA, 20 bp, Mouse
gagagatggcagacagaggc

SEQ ID NO: 532 - DNA, 20 bp, Mouse
agctctctgtccctggtgaa

SEQ ID NO: 533 - DNA, 20 bp, Mouse
tgccaaccactagcctctct

SEQ ID NO: 534 - DNA, 20 bp, Mouse
ctgaaccctccactctcctg

SEQ ID NO: 535 - DNA, 20 bp, Mouse
agccagggctacacagagaa

SEQ ID NO: 536 - DNA, 20 bp, Mouse
agccagggctacacagagaa

SEQ ID NO: 537 - DNA, 20 bp, Mouse
accctgctacaacgcagact

SEQ ID NO: 538 - DNA, 20 bp, Mouse
gcaagtttcaggagctaggg

SEQ ID NO: 539 - DNA, 20 bp, Mouse
ccccagaaccagagaccata

SEQ ID NO: 540 - DNA, 20 bp, Mouse
ccccagaaccagagaccata

SEQ ID NO: 541 - DNA, 20 bp, Mouse
ctaggggactctgccaagtg

SEQ ID NO: 542 - DNA, 20 bp, Mouse
caagacacccagtcccaact

SEQ ID NO: 543 - DNA, 20 bp, Mouse
tacttcccctttcccgaact

SEQ ID NO: 544 - DNA, 20 bp, Mouse
tccttggtgcttaccctcac

SEQ ID NO: 545 - DNA, 20 bp, Mouse
tgttcctgagttcacaacgc

SEQ ID NO: 546 - DNA, 20 bp, Mouse
attcccagcaactacatggc

SEQ ID NO: 547 - DNA, 20 bp, Mouse
acatgtccactgtggcaaaa

SEQ ID NO: 548 - DNA, 20 bp, Mouse
tgtcatgagtttgaggccag

SEQ ID NO: 549 - DNA, 20 bp, Mouse
atcagacagcccacaacctc

SEQ ID NO: 550 - DNA, 20 bp, Mouse
tatgtgccaccacacctgtc

SEQ ID NO: 551 - DNA, 20 bp, Mouse
gctcaaggaaggacacacct

SEQ ID NO: 552 - DNA, 22 bp, Mouse
tgctcttaacattttgagccat

SEQ ID NO: 553 - DNA, 20 bp, Mouse
gctcagcccctgaatcaata

SEQ ID NO: 554 - DNA, 20 bp, Mouse
gggatctgcctgtcttacca

SEQ ID NO: 555 - DNA, 20 bp, Mouse
ggaaggtagggcctggtaat

SEQ ID NO: 556 - DNA, 20 bp, Mouse
gctccaagatctgtgcgatt

SEQ ID NO: 557 - DNA, 20 bp, Mouse
ttagcgttagggtgagggtg

SEQ ID NO: 558 - DNA, 20 bp, Mouse
ggagactacggacttgtggc

SEQ ID NO: 559 - DNA, 20 bp, Mouse
cagttcttcccgaaaaccac

SEQ ID NO: 560 - DNA, 20 bp, Mouse
tttctgggaactgagatggc

SEQ ID NO: 561 - DNA, 20 bp, Mouse
gttggggctgctcatagaaa

SEQ ID NO: 562 - DNA, 20 bp, Mouse
gctgtggctctcttggagtt

SEQ ID NO: 563 - DNA, 20 bp, Mouse
ctctgatttcccacatgcct

SEQ ID NO: 564 - DNA, 20 bp, Mouse
aagagggagcactgaggaca

SEQ ID NO: 565 - DNA, 20 bp, Mouse
cagcagcaaatgacctttca

SEQ ID NO: 566 - DNA, 20 bp, Mouse
gaggcaggcagatttctgag

SEQ ID NO: 567 - DNA, 20 bp, Mouse
gtttcacatgttgtggtggc

SEQ ID NO: 568 - DNA, 20 bp, Mouse
gggacctttgggatagcatt

SEQ ID NO: 569 - DNA, 20 bp, Mouse
tcagacatctctggcctcct

SEQ ID NO: 570 - DNA, 20 bp, Mouse
ttcactaagttgcccaggct

SEQ ID NO: 571 - DNA, 22 bp, Mouse
tgcctttttctcacattgtctc

SEQ ID NO: 572 - DNA, 20 bp, Mouse
ttagaagcagaggcagaggc

SEQ ID NO: 573 - DNA, 20 bp, Mouse
gacctttggaagagcagtcg

SEQ ID NO: 574 - DNA, 20 bp, Mouse
tggcagctcacaatgtcttt

SEQ ID NO: 575 - DNA, 20 bp, Mouse
ggtgtggtgtaggggaagaa

SEQ ID NO: 576 - DNA, 22 bp, Mouse
tttcaactgcaaacacaaacag

SEQ ID NO: 577 - DNA, 19 bp, Mouse
agggccaaggaaggagaat

SEQ ID NO: 578 - DNA, 24 bp, Mouse
gcaaatatatagggtaccgagctg

SEQ ID NO: 579 - DNA, 20 bp, Mouse
cagattctccagctgtcagg

SEQ ID NO: 580 - DNA, 19 bp, Mouse
ctgtgtttccgcaccaagt

SEQ ID NO: 581 - DNA, 20 bp, Mouse
ctgcccgtccttatcttctg

SEQ ID NO: 582 - DNA, 20 bp, Mouse
acgcacgctcactcatacac

SEQ ID NO: 583 - DNA, 20 bp, Mouse
cagcagaggtgatgggttct

SEQ ID NO: 584 - DNA, 22 bp, Mouse
ttgtcacacagtggttaaatgc

SEQ ID NO: 585 - DNA, 20 bp, Mouse
tagaaccgtggctgaggact

SEQ ID NO: 586 - DNA, 24 bp, Mouse
ccgtaagatatgaaagaacttgga

SEQ ID NO: 587 - DNA, 20 bp, Mouse
taatcctggcttagcgcttg

SEQ ID NO: 588 - DNA, 20 bp, Mouse
tagaaagcacaggggacagg

SEQ ID NO: 589 - DNA, 20 bp, Mouse
ccttcctcgtctgagctgtt

SEQ ID NO: 590 - DNA, 20 bp, Mouse
ttgggacgtgacctgagaat

SEQ ID NO: 591 - DNA, 20 bp, Mouse
tatgtgtctggccgttgttc

SEQ ID NO: 592 - DNA, 19 bp, Mouse
gatgtgggtgcaggtgaag

SEQ ID NO: 593 - DNA, 20 bp, Mouse
ccccttctggagtgtctgaa

SEQ ID NO: 594 - DNA, 21 bp, Mouse
tctaggcagggctaccttttt

SEQ ID NO: 595 - DNA, 19 bp, Mouse
gctgagcagcctctagcaa

SEQ ID NO: 596 - DNA, 20 bp, Mouse
accatggcttttcccagtaa

SEQ ID NO: 597 - DNA, 20 bp, Mouse
ctgtgcctttggtgatcaga

SEQ ID NO: 598 - DNA, 20 bp, Mouse
tgtggcactctacggcataa

SEQ ID NO: 599 - DNA, 23 bp, Mouse
tgcatcactattaagcctcaacc

SEQ ID NO: 600 - DNA, 23 bp, Mouse
aagaatttgcaaagactgtgaga

SEQ ID NO: 601 - DNA, 20 bp, Mouse
ctggacctttggaagagcag

SEQ ID NO: 602 - DNA, 20 bp, Mouse
ggtggctcaaaccatccata

SEQ ID NO: 603 - DNA, 20 bp, Mouse
gagggcaatgagcaaaatgt

SEQ ID NO: 604 - DNA, 20 bp, Mouse
ggtcctgtctctggttcagg

SEQ ID NO: 605 - DNA, 20 bp, Mouse
taacacccacatcaggcaac

SEQ ID NO: 606 - DNA, 22 bp, Mouse
tttcatttcctggtgttccttt

SEQ ID NO: 607 - DNA, 20 bp, Mouse
aaacacaggcggaacgatag

SEQ ID NO: 608 - DNA, 20 bp, Mouse
ctatcgttccgcctgtgttt

SEQ ID NO: 609 - DNA, 21 bp, Mouse
aaggaagaggatggagaaaga

SEQ ID NO: 610 - DNA, 20 bp, Mouse
cgggtcttaatggagcagag

SEQ ID NO: 611 - DNA, 20 bp, Mouse
tcctccccagttacctagca

SEQ ID NO: 612 - DNA, 19 bp, Mouse
cagcaggcaagatgacctc

SEQ ID NO: 613 - DNA, 20 bp, Mouse
gtccctcaccagccatgtta

SEQ ID NO: 614 - DNA, 20 bp, Mouse
agcctgggctaagttgtgtg

SEQ ID NO: 615 - DNA, 20 bp, Mouse
tatgggccaatgttgttcct

SEQ ID NO: 616 - DNA, 20 bp, Mouse
atggtggctcacaaccatct

SEQ ID NO: 617 - DNA, 20 bp, Mouse
ttgtcctctgattgcagcat

SEQ ID NO: 618 - DNA, 20 bp, Mouse
cttgggtcatcaggctttgt

SEQ ID NO: 619 - DNA, 20 bp, Mouse
aagctgccctgctctctcta

SEQ ID NO: 620 - DNA, 20 bp, Mouse
atgctcagcctgctttgttt

SEQ ID NO: 621 - DNA, 20 bp, Mouse
gctgatagccctgggttcta

SEQ ID NO: 622 - DNA, 21 bp, Mouse
tgtacgcacaaattgacttgc

SEQ ID NO: 623 - DNA, 21 bp, Mouse
gaatccacattgcaaagccta

SEQ ID NO: 624 - DNA, 20 bp, Mouse
cacaggcaaatgaagggaag

SEQ ID NO: 625 - DNA, 20 bp, Mouse
ccagacttctccagctctcc

SEQ ID NO: 626 - DNA, 21 bp, Mouse
tcctcgagaggctctaggttt

SEQ ID NO: 627 - DNA, 20 bp, Mouse
tgcctagtcaaccacaggag

SEQ ID NO: 628 - DNA, 21 bp, Mouse
cctgtggttgactaggcagaa

SEQ ID NO: 629 - DNA, 20 bp, Mouse
gcctgatagcctggaataca

SEQ ID NO: 630 - DNA, 20 bp, Mouse
aaagggatgtgtggcgtaag

SEQ ID NO: 631 - DNA, 20 bp, Mouse
caaaacccaaccttctcagc

SEQ ID NO: 632 - DNA, 20 bp, Mouse
tgcactgaccgtgatagagg

SEQ ID NO: 633 - DNA, 20 bp, Mouse
cggtgtagctctggctgtct

SEQ ID NO: 634 - DNA, 20 bp, Mouse
catctcaccaactcgcactt

SEQ ID NO: 635 - DNA, 21 bp, Mouse
tttctgggaacaaagaggcta

SEQ ID NO: 636 - DNA, 20 bp, Mouse
gaacccaagtgttggggtaa

SEQ ID NO: 637 - DNA, 20 bp, Mouse
tggaagcccatctgtctctt

SEQ ID NO: 638 - DNA, 20 bp, Mouse
aaatgcaagtgggtgcttct

SEQ ID NO: 639 - DNA, 19 bp, Mouse
ccagaagagggcgtcagat

SEQ ID NO: 640 - DNA, 20 bp, Mouse
ggtgtgcaccaccatattca

SEQ ID NO: 641 - DNA, 21 bp, Mouse
gggaattatcagccaaaaagc

SEQ ID NO: 642 - DNA, 20 bp, Mouse
gcccaactgaaagctcaact

SEQ ID NO: 643 - DNA, 21 bp, Mouse
ggaagggggataacaattgaa

SEQ ID NO: 644 - DNA, 23 bp, Mouse
tgctaatttcaagcacagtgaga

SEQ ID NO: 645 - DNA, 20 bp, Mouse
agcttgacaccttgacagca

SEQ ID NO: 646 - DNA, 20 bp, Mouse
aacctgcagagaggagacca

SEQ ID NO: 647 - DNA, 20 bp, Mouse
ctccaaggggaggactcatt

SEQ ID NO: 648 - DNA, 24 bp, Mouse
ttcaattgagtttctctcctctga

SEQ ID NO: 649 - DNA, 20 bp, Mouse
tgcaggaccaagaagtaggc

SEQ ID NO: 650 - DNA, 20 bp, Mouse
cgagatctgatgccctcttc

SEQ ID NO: 651 - DNA, 20 bp, Mouse
tgctgagagcagaaaaggaa

SEQ ID NO: 652 - DNA, 166 bp, Mouse
  misc_feature 106: At position 106 ′n′ equals c, t, a or g
gcagtgagctgcagagtttgcagaatgagggcactctaaactcatcaagtgaggaggcccttccctcacactccagatggctgataggtggcattacatggtccancgcgcgcacgcgctcagatgcaatctccacattcataaccagatgtccttgggtaggcct

(19) United States
(12) Patent Application Publication
(10) Pub. No.: US 2004/0081964 A1
(43) Pub. Date: Apr. 29, 2004

(54) Gene and sequence variation associated with sensing carbohydrate compounds and other sweeteners

(76) Inventors: Alexander A. Bachmanov, Philadelphia, PA (US); Gary K. Beauchamp, Philadelphia, PA (US); Shannu Li, Philadelphia, PA (US); Xia Li, Philadelphia, PA (US); Danielle R. Reed, Philadelphia, PA (US); Michael Guy Tordoff, Philadelphia, PA (US); David A. Ross, San Rafael, CA (US); Jeffrey D. Ohman, Alameda, CA (US); Aurobindo Chatterjee, Ann Arbor, MI (US); Pieter J. De Jong, Foster City, CA (US)

Correspondence Address: PFIZER INC., PATENT DEPARTMENT, MS8260-1611, EASTERN POINT ROAD, GROTON, CT, 06340, US

(21) Appl. No.: 10/280,183

(22) Filed: Oct. 25, 2002

(51) Int. Cl. C12Q 1/68; A01K 67/027; C07H 21/04; C07K 14/705

(52) U.S. Cl. 435/6; 435/69.1; 435/320.1; 435/325; 530/350; 536/23.5; 800/18

(57) ABSTRACT

The present invention relates to the discovery of a gene and its sequence variation associated with preference for carbohydrates, other sweet compounds, or ethanol. The present invention also relates to the study of metabolic pathways to identify other genes, receptors, and relationships that contribute to differences in sensing of carbohydrates or ethanol. The present invention also relates to germline or somatic sequence variations and its use in the diagnosis and prognosis of predisposition to diabetes, other obesity related disorders, or ethanol consumption. The present invention also provided probes or primers specific for the detection and analysis of such sequence variation. The present invention also relates to method for screening drugs for inhibition or restoration of gene function as antidiabetic, antiobesity, or antialcohol consumption therapies. The present invention relates to other antidiabetic, antiobesity disorder, or antialcohol consumption therapies, such as gene therapy, protein replacement therapy, etc. Finally, the present invention relates to a method for identifying sweeteners or alcohols utilizing the gene and its variations.

### FIELD OF THE INVENTION

The present invention relates generally to the field of mouse and human genetics and sensing of extracellular carbohydrates. Specifically, the present invention relates to the discovery of a gene and its sequence variation associated with a differential preference for sweet compounds in laboratory strains of mice.

### BACKGROUND OF THE INVENTION

The ability to sense extra-cellular carbohydrates, transduce this sensory information, and relay it to the brain, is carried out by membrane bound receptors in taste papillae. Many approaches to identify the sweet receptor or receptors have been tried, but the problem has proved, until recently, to be difficult.

Mammals vary in their ad libitum consumption of sweeteners. To investigate the genetic contribution to this complex behavior, behavioral, electrophysiological, and genetic studies were conducted using two strains of mice that differ markedly in their preference for sucrose and saccharin (Bachmanov et al., Behavior Genetics, 1996;26:563-573).

Recently published data indicates that the ability to sense carbohydrates is linked to obesity. These studies demonstrated that sensation of simple carbohydrates is suppressible by the adipose hormone, leptin.

These studies demonstrated that a locus on the telomere of mouse chromosome 4 accounts for ˜40% of the genetic variability in sucrose and saccharin intake, and that the effect of this locus is to enhance or retard the gustatory neural response to sucrose.

### SUMMARY OF THE INVENTION

The present invention provides a gene and its sequence variation associated with a preference for carbohydrate compounds, other sweeteners, or alcohol.

The present invention provides a gene and its sequence variation associated a differential response by the pancreas and/or muscle in response to dietary carbohydrates.

The present invention also relates to sequence variation and its use in the diagnosis and prognosis of predisposition to diabetes, other obesity-related disorders, or alcohol consumption.

The present invention also relates to the study of taste to identify molecules responsible for signal transduction, other receptors and genes and relationships that contribute to taste preference.

The present invention also relates to the study of diabetes to identify molecules responsible for sensing extra-cellular carbohydrate, other receptors and genes and relationships that contribute to a diabetic state.

The present invention also relates to a sequence variation and its use in the identification of specific alleles altered in their specificity for carbohydrate compounds.

The present invention also relates to a recombinant construct comprising SAC1 (also referred to as Sac) polynucleotide suitable for expression in a transformed host cell.

The present invention also provides primers and probes specific for the detection and analysis of the SAC1 locus.

The present invention also relates to kits for detecting a polynucleotide comprising a portion of the SAC1 locus.

The present invention also relates to transgenic animals, which carry an altered SAC1 allele, such as a knockout mouse.

The present invention also relates to methods for screening drugs for inhibition or restoration of SAC1 function as a taste receptor.

The present invention also relates to identification of sweeteners or alcohols using the SAC1 gene and its sequence variations.

The present invention also relates to methods for screening drugs for inhibition or restoration of SAC1 function in homeostatic regulation of glucose levels.

The present invention also relates to methods for screening drugs for modification of SAC1 function in the consumption of alcohol.

Finally, the present invention provides therapies directed to diabetic or obesity disorders. Therapies of diabetes and obesity include gene therapy, protein replacement, protein mimetics, and inhibitors.

### BRIEF DESCRIPTION OF THE FIGURES

FIG. 1A shows genetic mapping of the SAC1 locus, using 632 F2 mice from a cross between the B6 (high preference) and 129 (low preference) strains. Mapping results were obtained with MAPMAKER/QTL Version 1.1, using an unconstrained model. A black triangle at the bottom indicates peak LOD score at M134G01 marker. Horizontal line at the bottom shows a 1-LOD confidence interval.

FIG. 1B shows SAC1-containing chromosomal region defined by a donor fragment of the 129.B6-Sac^{b} partially congenic mice. The partially congenic strains were constructed by identifying several founder F2 mice with small fragments of the telomeric region of mouse chromosome 4 from the B6 strain and successive backerossing to the 129 strain. Presence and size of donor fragment were determined by genotyping polymorphic markers in mice from the N4, N6, N7, N4F4, and N3F5 generations.

FIG. 1C shows average daily saccharin consumption by N6, N7, N4F4, and N3F5 segregating partially congenic 129.B6-Sac mice in 4-days two-bottle tests with water (means±SE). The open bar indicates intakes of mice that did not inherit the donor fragment. The black bar indicates intakes of mice with one or two copies of the donor fragment, which is flanked by 280G12-T7 proximally and D4Mon1 distally. The complete donor fragment is represented by overlapping sequences of the BAC RPCI-23-118E21 and a genomic clone (Accession AF185591), as indicated at the bottom. The size of the SAC1-containing donor fragment is 194, 478 kb.

FIG. 1D shows BAC contig of distal chromosome 4 in the SAC1 region. Using ³²P radioactively labeled probes from the nonrecombinant interval, a mouse BAC library (RPCI-23) was screened; positive clones were confirmed by PCR analysis and only clones positive by hybridization and by PCR are included in the contig. BAC ends were sequenced and PCR primers designed. The STS content of each BAC, using all BAC ends was determined. BAC size was determined by digesting the BAC with NotI, and the insert size determined using pulse field gel electrophoresis.

FIG. 1E shows genes contained within the SAC1 nonrecombinant interval. Arrows indicate predicted direction of transcription. See Table 1 for a description of gene prediction, and details concerning function.

FIG. 2A shows the mouse SAC1 gene (mSac; Accession AF311386), its human ortholog (hSac), and the previously described gene T1R1, now Gpr70, are aligned above. Residues shaded in black are identical between at least two identical residues; residues in gray indicate conservative changes. The human ortholog was identified by sequence homology search within the htgs database (Accession AC026283). The amino acid sequence of the human ortholog was predicted using GENSCAN. The amino acid sequence of mouse Gpr70 was obtained by constructing primers based upon the nucleotide sequence, and taste cDNA was amplified and sequenced. This amino acid and nucleotide sequence for Gpr70 differed slightly from the initial report; the sequence reported in this paper has been deposited in GenBank (AF301161, AF301162). The location of the missense mutation is indicated by an *.

FIG. 2B shows structure of the SAC1 gene. The six exons are shown as black boxes.

FIG. 2C shows conformation of a protein predicted from the Sac gene. To determine the transmembrane regions, the hydrophobicity was determined using the computer program HMMTOP, and drawn with TOPO. The missense mutation is denoted with an asterisk.

FIG. 3 shows saccharin and sucrose preferences by mice from inbred strains with two different haplotypes of the Sac gene. The haplotype found in the B6 mice and the other high sweetener-preferring inbred strains consisted of four variants, two variants were 5′ of the predicted translation start codon, one variant was a missense mutation (Ile61Thr), and the last variant was located in the intron between exon 2 and 3. The strains with the B6-like haplotype of Sac strongly preferred saccharin (82±4%) and sucrose (86±6%), whereas strains with the 129-like haplotype were indifferent to these solutions (57±2% and 54±1% respectively, p=0.0015).

FIG. 4A shows tissue expression of the SAC1 gene. Note that cDNA was obtained from a commercial source for the multiple tissue panel, with the exception of tongue cDNA, which was as isolated by the investigator, as described within the text. Relative band intensities may differ due to differences in cDNA isolation methods or concentration.

FIG. 4B shows RNA from human fungiform papillae was obtained from biopsy material, reversed transcribed, and the resulting bands from genomic and cDNA were amplified using primers, described in the text. The bands were excised from the agarose gel, purified and reamplified. The PCR product was sequenced to confirm that the bands amplified the human otholog to Sac.

FIG. 5 shows amino acid sequence alignment of the mouse cDNA sequence for the SAC1 gene and the cDNA for a calcium sensing metabotropic receptor. Dark areas indicated regions of shared similarity.

FIG. 6 plots the hydrophobicity of the SAC1 amino acid sequence as predicted by the computer program Top Pred. Note the seven transmembrane domains characteristic of G-protein coupled receptors.

### DETAILED DESCRIPTION OF THE INVENTION

### I. Definitions

The present invention employs the following definitions:

As used herein, the terms “polynucleotide” and “nucleic acid” refer to naturally occurring polynucleotides, e.g., DNA or RNA. These terms do not refer to a specific length. Thus, these terms include oligonucleotide, primer, probe, etc. These terms also refer to analogs of naturally occurring polynucleotides. The polynucleotide may be double stranded or single stranded. The polynucleotides may be labeled with radiolabels, fluorescent labels, enzymatic labels, proteins, haptens, antibodies, sequence tags.

For example, these terms include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “polynucleotide amplification” refers to a broad range of techniques for increasing the number of copies of specific polynucleotide sequences. Typically, amplification of either or both strand of the target nucleic acid comprises the use of one or more nucleic acid-modifying enzymes, such as a DNA polymerase, a ligase, an RNA polymerase, or an RNA-dependent reverse transcriptase. Examples of polynucleotide amplification reaction include, but not limited to, polymerase chain reaction (PCR), nucleic acid sequence based amplification (NASB), self-sustained sequence replication (3SR), strand displacement activation (SDA), ligase chain reaction (LCR), Qβ replicase system, and the like.

As used herein, the term “primer” refers to a nucleic acid, e.g., synthetic polynucleotide, which is capable of annealing to a complementary template nucleic acid (e.g., the SAC1 locus) and serving as a point of initiation for template-directed nucleic acid synthesis. A primer need not reflect the exact sequence of the template but must be sufficiently complementary to hybridize with a template. Typically, a primer will include a free hydroxyl group at the 3′ end. The appropriate length of a primer depends on the intended use of the primer but typically ranges from 12 to 30 nucleotides. The term primer pair means a set of primers including a 5′ upstream primer that hybridizes with the 5′ end of the target sequence to be amplified and a 3′ downstream primer that hybridizes with the complement of the 3′ end of the target sequence to be amplified.

The present invention includes all novel primers having at least eight nucleotides derived from the SAC1 locus for amplifying the SAC1 gene, its complement or functionally equivalent nucleic acid sequences. The present invention does not include primers which exist in the prior art. That is, the present invention includes all primers having at least 8 nucleotides with the proviso that it does not include primers existing in the prior art.

“Target polynucleotide” refers to a single- or double-stranded polynucleotide which is suspected of containing a target sequence, and which may be present in a variety of types of samples, including biological samples.

“Antibody” refers to polyclonal and/or monoclonal antibody and fragments thereof, and immunologic binding equivalents thereof, which are capable of specifically binding to the SAC1 polypeptides and fragments thereof or to polynucleotide sequences from the SAC1 region, particularly from the SAC1 locus or a portion thereof. Antibody may be a homogeneous molecular entity, or a mixture such as a serum product made up of a plurality of different molecular entities.

Antibodies may be produced by in vitro or in vivo techniques well-known in the art. For example, for production of polyclonal antibodies, an appropriate target immune system, typically mouse or rabbit, is selected. Substantially purified antigen is presented to the immune system. Typical sites for injection are in footpads, intramuscularly, intraperitoneally, or intradermally. Polyclonal antibodies may then be purified and tested for immunological response, e.g., using an immunoassay.

For production of monoclonal antibodies, protein, polypeptide, fusion protein, or fragments thereof may be injected into mice. After the appropriate period of time, the spleens may be excised and individual spleen cells fused, typically, to immortalized myeloma cells under appropriate selection conditions. Thereafter, the cells are clonally separated and the supernatants of each clone tested for their production of an appropriate antibody specific for the desired region of the antigen. Affinities of monoclonal antibodies are typically 10⁻⁸ M⁻¹ or preferably 10⁻⁹ to 10⁻¹⁰ M⁻¹ or stronger.

Other suitable techniques involve in vitro exposure of lymphocytes to the antigenic polypeptides, or alternatively, to selection of libraries of antibodies in phage or similar vectors.

Frequently, antibodies are labeled by joining, either covalently or non-covalently, a substance which provides for a detectable signal. A wide variety of labels and conjugation techniques are known. Suitable labels include radionuclides, enzymes, substrates, cofactors, inhibitors, fluorescent agents, chemiluminescent agents, magnetic particles, and the like. Also, recombinant immunoglobulins may be produced.

“Binding partner” refers to a molecule capable of binding another molecule with specificity, as for example, an antigen and an antigen-specific antibody or an enzyme and its inhibitor. Binding partners are known in the art and include, for example, biotin and avidin or streptavidin, IgG and protein A, receptor-ligand couples, and complementary polynucleotide strands. In the case of complementary polynucleotide binding partners, the partners are normally at least about 15, 20, 25, 30, 40 bases in length.

A “biological sample” refers to a sample of tissue or fluid suspected of containing an analyte (e.g., polynucleotide, polypeptide) including, but not limited to, e.g., plasma, serum, spinal fluid, lymph fluid, the external sections of the skin, respiratory, intestinal, and genitourinary tracts, tears, saliva, blood cells, organs, tissue and samples of in vitro cell culture constituents. A biological sample is typically from human or other animal.

“Encode.” A polynucleotide is said to “encode” a polypeptide if, in its native state or when manipulated by methods well-known to those skilled in the art, it can be transcribed and/or translated to produce the mRNA and/or the polypeptide or a fragment thereof. The antisense strand is the complement of such a nucleic acid, and the encoding sequence can be deduced therefrom.

“Isolated” or “substantially pure” polynucleotide or polypeptide (e.g., an RNA, DNA, protein) is one which is substantially separated from other cellular components which naturally accompany a native human nucleic acid or protein, e.g., ribosomes, polymerases, many other human genome sequences and proteins. The term embraces a nucleic acid or peptide sequence which has been removed from its naturally occurring environment, and includes recombinant or cloned DNA isolates and chemically synthesized analogs or analogs biologically synthesized by heterologous systems.

“SAC1 Allele” refers to normal alleles of the SAC1 locus as well as alleles carrying variations that predispose individuals to develop obesity, diabetes, or for alcohol consumption or alcoholism.

“SAC1 Locus” refers to polynucleotides, which are in the SAC1 region, that are likely to be expressed in normal individual, certain alleles of which predispose an individual to develop obesity, diabetes, or alcohol consumption or alcoholism. The SAC1 locus includes coding sequences, intervening sequences and regulatory elements controlling transcription and/or translation. The SAC1 locus includes all allelic variations of the DNA sequence.

The DNA sequences used in this invention will usually comprise at least about 5 codons (15 nucleotides), 7, 10, 15, 20, or 30 codons, and most preferably, at least about 35 codons. One or more introns may also be present. This number of nucleotides is usually about the minimal length required for a successful probe that would hybridize specifically with a SAC1 locus.

“SAC1 Region” refers to a portion of mouse chromosome 4 bounded by the markers 280G12-T7 and D4Mon1 GenBank Accession number is YG7772 (SEQ ID NO: 652) and is GCAGTGAGCTGCAGAGTTTGCAGAATGAGGGCACTCTAAACTCATCAA GTGAGGAGGCCCTTCCCTCACACTCCAGATGGCTGATAGGTGGCATTA CATGGTC(CA)nCGCGCGCACGCGCTCAGATGCAATCTCCACATTCATA ACCAGATGTCCTTGGGTAGGCCT. The CA sequence in the middle is variable in length. In the B6 mouse, n=19, while in the 129 mouse, n=16. This region contains the SAC1 locus, including the SAC1 gene. GenBank accession number for the SAC1 gene is AF311386.

As used herein, a “portion” or “fragment” of the SAC1 gene, locus, region, or allele is defined as having a minimal size of at least about 15 nucleotides, or preferably at least about 20, or more preferably at least about 25 nucleotides, and may have a minimal size of at least about 40 nucleotides.

As used herein, the term “polypeptide” refers to a polymer of amino acids without referring to a specific length. This term includes to naturally occurring protein. The term also refers to modifications, analogues and functional mimetics thereof. For example, modifications of the polypeptide may include glycosylations, acetylations, phosphorylations, and the like. Analogues of polypeptide include unnatural amino acid, substituted linkage, etc. Also included are polypeptides encoded by DNA which hybridize under high or low stringency conditions, to the nucleic acids of interest.

Modification of polypeptides includes those substantially homologous to primary structural sequence, e.g., in vivo or in vitro chemical and biochemical modifications or incorporation unusual amino acids. Such modifications include, for example, acetylation, carboxylation, phosphorylation, glycosylation, ubiquitination, labeling, e.g., with radionuclides, and various enzymatic modifications, as will be readily appreciated by those well-skilled in the art. A variety of methods for labeling polypeptides and of substituents or labels useful for such purposes are well-known in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well-known in the art (see Sambrook et al., 1989 or Ausubel et al., 1992).

Besides substantially full-length polypeptides, the present invention provides for biologically active fragments of the polypeptides. Significant biological activities include ligand-binding, immunological activity, and other biological activities characteristic of SAC1 polypeptides. Immunological activities include both immunogenic function in a target immune system, as well as sharing of immunological epitopes for binding, serving as either a competitor or substitute antigen for an epitope of the SAC1 protein. As used herein, “epitope” refers to an antigenic determinant of a polypeptide. An epitope could comprise three amino acids in a spatial conformation that is unique to the epitope. Generally, an epitope consists of at least five such amino acids, and more usually consists of at least 8 to 10 such amino acids. Methods of determining the spatial conformation of such amino acids are known in the art.

For immunological purposes, tandem-repeat polypeptide segments may be used as immunogens, thereby producing highly antigenic proteins. Alternatively, such polypeptides will serve as highly efficient competitors for specific binding.

Fusion proteins comprise SAC1 polypeptides and fragments. Homologous polypeptides may be fusions between two or more SAC1 polypeptide sequences or between the sequences of SAC1 and a related protein. Likewise, heterologous fusions may be constructed which would exhibit a combination of properties or activities of the derivative proteins. For example, ligand-binding or other domains may be “swapped” between different new fusion polypeptides or fragments. Such homologous or heterologous fusion polypeptides may display, for example, altered strength or specificity of binding. Fusion partners include immunoglobulins, bacterial β-galactosidase, trpE, protein A, β-lactamase, α-amylase, alcohol dehydrogenase, and yeast a mating factor.

Fusion proteins will typically be made by either recombinant nucleic acid methods or may be chemically synthesized. Techniques for the synthesis of polypeptides are known in the art.

Functional mimetics of a native polypeptide may be obtained using known methods in the art. For example, polypeptides may be least about 50% homologous to the native amino acid sequence, preferably in excess of about 70%, and more preferably at least about 90% homologous. Substitutions typically contain the exchange of one amino acid for another at one or more sites within the polypeptide, and may be designed to modulate one or more properties of the polypeptide, such as stability against proteolytic cleavage, without the loss of other functions or properties. Amino acid substitutions may be made on the basis of similarity in polarity, charge, solubility, hydrophobicity, hydrophilicity, and/or the amphipathic nature of the residues involved. Preferred substitutions are ones which are conservative, that is, one amino acid is replaced with one of similar shape and charge. Conservative substitutions are well-known in the art and typically include substitutions within the following groups: glycine, alanine; valine, isoleucine, leucine; aspartic acid, glutamic acid; asparagine, glutamine; serine, threonine; lysine, arginine; and tyrosine, phenylalanine.

Certain amino acids may be substituted for other amino acids in a polypeptide structure without appreciable loss of interactive binding capacity with structures such as, for example, antigen-binding regions of antibodies or binding sites on substrate molecules or binding sites on proteins interacting with a polypeptide. Since it is the interactive capacity and nature of a polypeptide which defines that polypeptide's biological functional activity, certain amino acid substitutions can be made in a protein sequence, and its underlying DNA coding sequence, and nevertheless obtain a protein with like properties. In making such changes, the hydropathic index of amino acids may be considered. The importance of the hydrophobic amino acid index in conferring interactive biological function on a protein is generally understood in the art. Alternatively, the substitution of like amino acids can be made effectively on the basis of hydrophilicity.

A peptide mimetic may be a peptide-containing molecule that mimics elements of protein secondary structure. The underlying rationale behind the use of peptide mimetics is that the peptide backbone of proteins exists chiefly to orient amino acid side chains in such a way as to facilitate molecular interactions, such as those of antibody and antigen, enzyme and substrate or scaffolding proteins. A peptide mimetic is designed to permit molecular interactions similar to the natural molecule. A mimetic may not be a peptide at all, but it will retain the essential biological activity of a natural polypeptide.

Polypeptides may be produced by expression in a prokaryotic cell or produced synthetically. These polypeptides typically lack native post-translational processing, such as glycosylation. Polypeptides may be labeled with radiolabels, fluorescent labels, enzymatic labels, proteins, haptens, antibodies, sequence tags. “SAC1 polypeptide” refers to a protein or polypeptide encoded by the SAC1 locus, variants, fragments or functional mimics thereof. A SAC polypeptide may be that derived from any of the exons described herein which may be in isolated and/or purified form. The length of SAC1 polypeptide sequences is generally at least about 5 amino acids, usually at least about 10, 15, 20, 30 residues.

“Alcohol consumption” relates to the intake and/or preference of an animal for ethanol.

“Diabetes” refers to any disorder that exhibits phenotypic features of an increased or decreased level of a biological substance associated with glucose or fatty acid metabolism. The term “carbohydrate” refers to simple mono and disaccharides.

The terms “sequence variation” or “variant form” encompass all forms of polymorphism and mutations. A sequence variation may range from a single nucleotide variation to the insertion, modification, or deletion of more than one nucleotide. A sequence variation may be located at the exon, intron, or regulatory region of a gene.

Polymorphism refers to the occurrence of two or more genetically determined alternative sequences or alleles in a population. A biallelic polymorphism has two forms. A triallelic polymorphism has three forms. A polymorphic site is the locus at which sequence divergence occurs. Diploid organisms may be homozygous or heterozygous for allelic forms. Polymorphic sites have at least two alleles, each occurring at frequency of greater than 1% of a selected population. Polymorphic sites also include restriction fragment length polymorphisms, variable number of tandem repeats (VNTRs), hypervariable regions, minisatellites, dinucleotide repeats, trinucleotide repeats, tetranucleotide repeats, simple sequence repeats, and insertion elements. The first identified allelic form may be arbitrarily designated as the reference sequence and other allelic forms may be designated as alternative or variant alleles. The allelic form occurring most frequently in a selected population is sometimes referred to as the wild type form or the consensus sequence.

Mutations include deletions, insertions and point mutations in the coding and noncoding regions. Deletions may be of the entire gene or of only a portion of the gene. Point mutations may result in stop codons, frameshift mutations, or amino acid substitutions. Somatic mutations are those which occur only in certain tissues, such as liver, heart, etc. and are not inherited in the germline. Germline mutations can be found in any of a body's tissues and are inherited.

“Operably linked” refers to a juxtaposition wherein the components are in a relationship permitting them to function in their intended manner. For instance, a promoter is operably linked to a coding sequence if the promoter affects its transcription or expression.

The term “probes” refers to polynucleotide of any suitable length which allows specific hybridization to the target region. Probes may be attached to a label or reporter molecule using known methods in the art. Probes may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about 8 nucleotides, usually at least about 15 nucleotides, and fewer than about 6 kb, usually fewer than about 1.0 kb, from a polynucleotide sequence encoding SAC1 are preferred as probes.

The terms “isolated,” “substantially pure,” and “substantially homogeneous” are used interchangeably to describe a protein or polypeptide which has been separated from components which accompany it in its natural state. A monomeric protein is substantially pure when at least about 60% to 75% of a sample exhibits a single polypeptide sequence. A substantially pure protein will typically comprise about 60% to 90% W/W of a protein sample, more usually about 95%, and preferably will be over about 99% pure. Protein purity or homogeneity may be indicated by a number of means well-known in the art, such as polyacrylamide gel electrophoresis of a protein sample, followed by visualizing a single polypeptide band upon staining the gel. For certain purposes, higher resolution may be provided by using HPLC or other means well-known in the art which are utilized for purification.

A SAC1 protein is substantially free of naturally associated components when it is separated from the native contaminants which accompany it in its natural state. Thus, a polypeptide which is chemically synthesized or synthesized in a cellular system different from the cell from which it naturally originates will be substantially free from its naturally associated components. A protein may also be rendered substantially free of naturally associated components by isolation, using protein purification techniques well-known in the art.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

“Substantial homology or similarity.” A nucleic acid or fragment thereof is of substantially homologous (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Identity means the degree of sequence relatedness between two polypeptide or two polynucleotides sequences as determined by the identity of the match between two strings of such sequences. Identity can be readily calculated (Lesk A. M., ed., Computational Molecular Biology, New York: Oxford University Press, 1988; Smith D. W., ed., Biocomputing: Informatics and Genome Projects, New York: Academic Press, New York, 1993; Griffin A. M., and Griffin H. G., eds., Computer Analysis of Sequence Data, Part 1, New Jersey: Humana Press, 1994; von Heinje G., Sequence Analysis in Molecular Biology, Academic Press, 1987; and Gribskov M. and Devereux J., eds., Sequence Analysis Primer, New York: M Stockton Press, 1991).

Alternatively, substantial homology or similarity exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about 9 nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter.

The terms “substantial homology” or “substantial identity,” when referring to polypeptides, indicate that the polypeptide or protein in question exhibits at least about 30% identity with an entire naturally-occurring protein or a portion thereof, usually at least about 70% identity, and preferably at least about 95% identity.

Homology, for polypeptides, is typically measured using sequence analysis software (see, e.g., the Sequence Analysis Software Package of the Genetics Computer Group, University of Wisconsin Biotechnology Center). Protein analysis software matches similar sequences using measures of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

“Substantially similar function” refers to the function of a modified nucleic acid or a modified protein, with reference to the wild-type SAC1 nucleic acid or wild-type SAC1 polypeptide. The modified polypeptide will be substantially homologous to the wild-type SAC1 polypeptide and will have substantially the same function. The modified polypeptide may have an altered amino acid sequence and/or may contain modified amino acids. In addition to the similarity of function, the modified polypeptide may have other useful properties, such as a longer half-life. The similarity of function (activity) of the modified polypeptide may be substantially the same as the activity of the wild-type SAC1 polypeptide. Alternatively, the similarity of function (activity) of the modified polypeptide may be higher than the activity of the wild-type SAC1 polypeptide. The modified polypeptide is synthesized using conventional techniques, or is encoded by a modified nucleic acid and produced using conventional techniques. The modified nucleic acid is prepared by conventional techniques. A nucleic acid with a function substantially similar to the wild-type SAC1 gene function produces the modified protein described above.

A polypeptide “fragment,” “portion,” or “segment” is a stretch of amino acid residues of at least about 5 to 7 contiguous amino acids, often at least about 7 to 9 contiguous amino acids, typically at least about 9 to 13 contiguous amino acids and, most preferably, at least about 20 to 30 or more contiguous amino acids.

The polypeptides of the present invention, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Target region” refers to a region of the nucleic acid which is amplified and/or detected. The term “target sequence” refers to a sequence with which a probe or primer will form a stable hybrid under desired conditions.

### II. Positional Cloning of Mouse SAC1 Gene and the Discovery of a Gene and Its Sequence Variation Associated With Altered Sensation for Carbohydrates

Inbred strains of mice differ in their intake of sweeteners (Bachmanov A. A., Reed D. R., Tordoff M. G., Price R. A., and Beauchamp G. K. Intake of ethanol, sodium chloride, sucrose, citric acid, and quinine hydrochloride solutions by mice: a genetic analysis. Behavior Genetics, 1996;26:563-573; Lush I. E., The genetics of tasting in mice. VI. Saccharin, acesulfame, dulcin and sucrose. Genet Res, 1989;53:95-99; Lush I. The genetics of bitterness, sweetness, and saltiness in strains of mice. In Genetics of Perception and Communication, Vol. 3, eds. Wysocki C. and Kare M., New York: Marcel Dekker, 1991:227-235; Capretta P. J. Saccharin and saccharin-glucose ingestion in two inbred strains of Mus musculus. Psychon. Sci., 1970;21:133-135; Nachman M. The inheritance of saccharin preference. Journal of Comp Physiol Psychol, 1959;52:451-457). Breeding and linkage experiments suggest that a single gene, the Sac locus (for saccharin intake), accounts for a large proportion of the genetic variance (Fuller J. L. Single-locus control of saccharin preference in mice. Journal of Heredity, 1974;65:33-36; Capeless C. G. and Whitney G. The genetic basis of preference for sweet substances among inbred strains of mice: preference ratio phenotypes and the alleles of the Sac and dpa loci. Chem Senses, 1995;20:291-298; Bachmanov A. A. et al. Sucrose consumption in mice: major influence of two genetic loci affecting peripheral sensory responses. Mammalian Genome, 1997;8:545-548; Belknap J. K. et al. Single-locus control of saccharin intake in BXD/Ty recombinant inbred (RI) mice: some methodological implications for RI strain analysis. Behav Genet, 1992;22:81-100; Blizard D. A., Kotlus B., and Frank M. E. Quantitative trait loci associated with short-term intake of sucrose, saccharin and quinine solutions in laboratory mice. Chem Senses, 1999;24:373-85). Using genetic and physical mapping methods, an interval of 194 kb was identified at the telomeric end of mouse chromosome 4 that contains the Sac locus. BAC sequencing within this interval led to the identification of a gene that has a 30% amino acid homology with other putative taste receptors (Hoon M. A. et al. Putative mammalian taste receptors: a class of taste-specific GPCRs with distinct topographic selectivity. Cell, 1999;96:541-551). This gene is expressed in mouse tongue. Mutation detection on this gene revealed a missense mutation (Ile61Thr) with four other sequence variants define a haplotype found in mice with low sweetener preference (129, Balb/c, AKR, and DBA2). An alternative five variant haplotype is found in mice with a high preference for sweet fluids (B6, SWR, IS, ST, and SEA). A human ortholog of this gene exists, and is expressed in human taste papillae. We therefore suggest that this gene is a sweet taste receptor, and variation within this gene is responsible for the phenotype of the Sac locus.

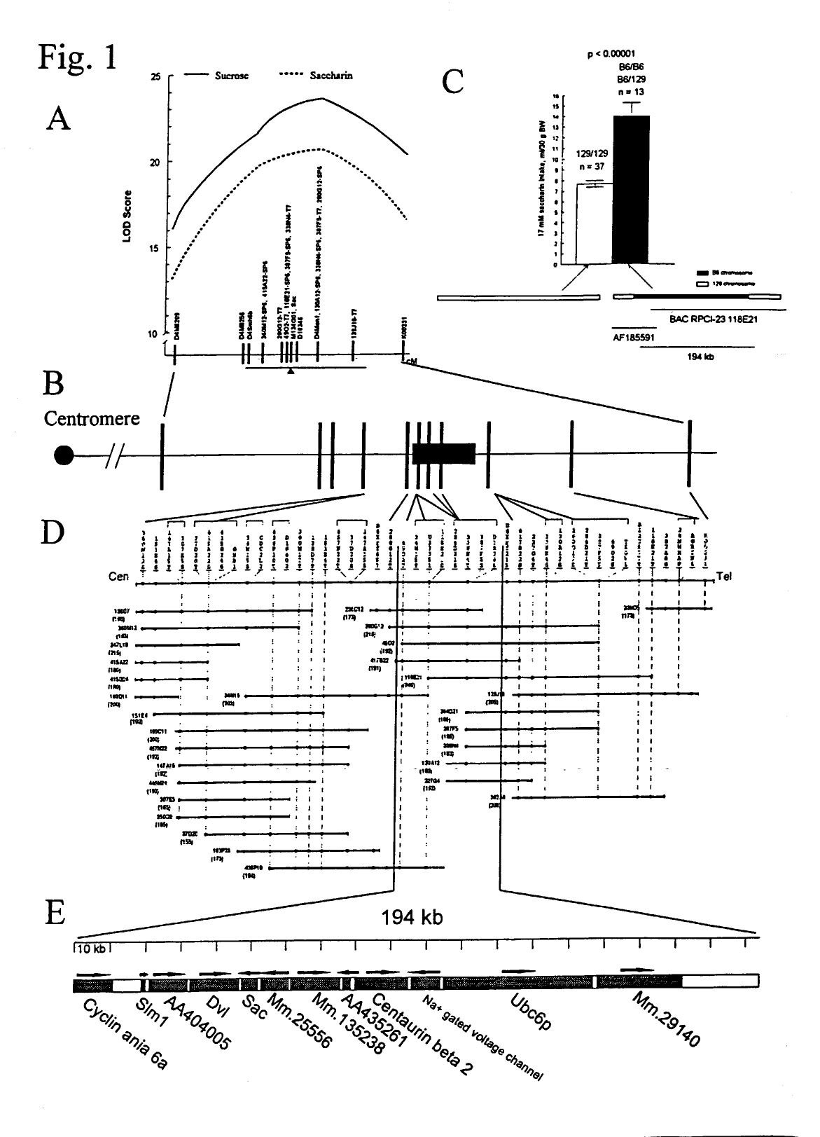

To identify this locus, mice from the high sweetener preference (C57BL/6ByJ; B6) and the low sweetener preference (129P3/J; formerly 129/J, abbreviated here as 129) were used as parental strains to produce an F2 generation. The F2 mice were phenotyped for sweetener preference using 96-hour two-bottle taste tests and genotyped with markers polymorphic between the B6 and 129 strains (FIG. 1A). The results of this analysis indicated peak linkage near marker D118346 with the B6 allele having a dominant mode of inheritance. Using recombinant mice from the F2 generation, 129.B6-Sac partially congenic mice were created, using genotypic (B6 allele at D18346; FIG. 1B) and phenotypic (high saccharin intake; FIG. 1C) characteristics as selection criteria for each generation. Genotyping of partially congenic mice with polymorphic markers defined the Sac nonrecombinant interval. Radiation hybrid mapping was conducted with additional markers (R74924, D18402, D18346, Agrin, V2r2 and D4Ertd296c). These markers were amplified using DNA and mouse and hamster control DNA in the T31 mouse radiation hybrid panel, scored for the presence or absence of an appropriately sized band, and the data analyzed by the Jackson Laboratory. All markers were within the SAC1 confidence interval suggested by the initial linkage analysis, and were used in subsequent analyses.

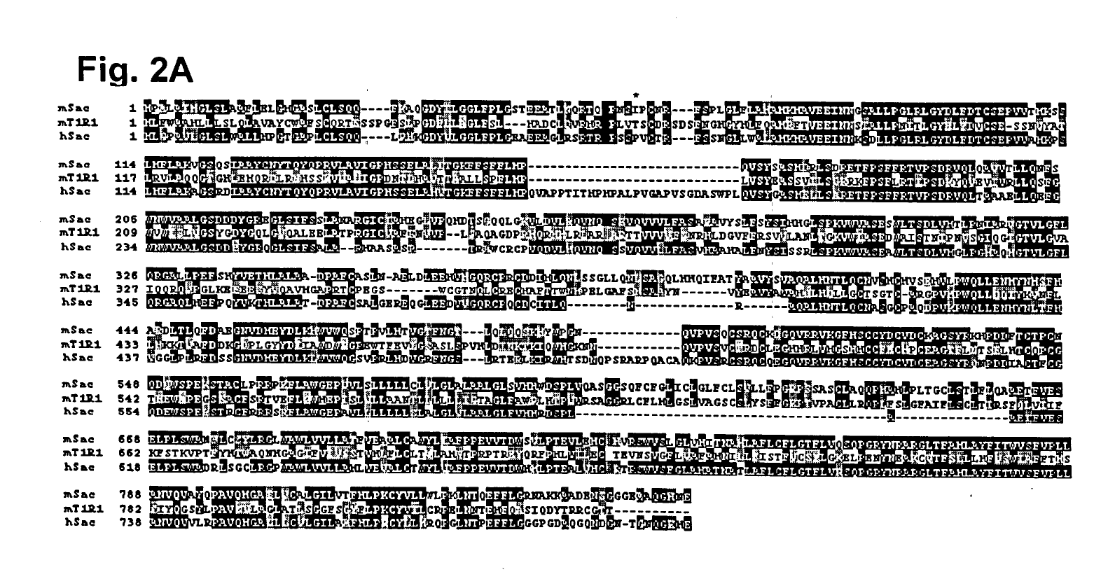

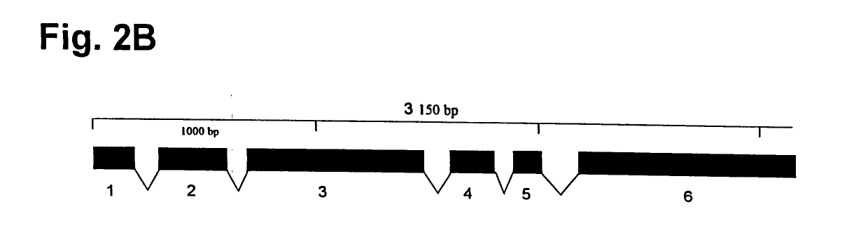

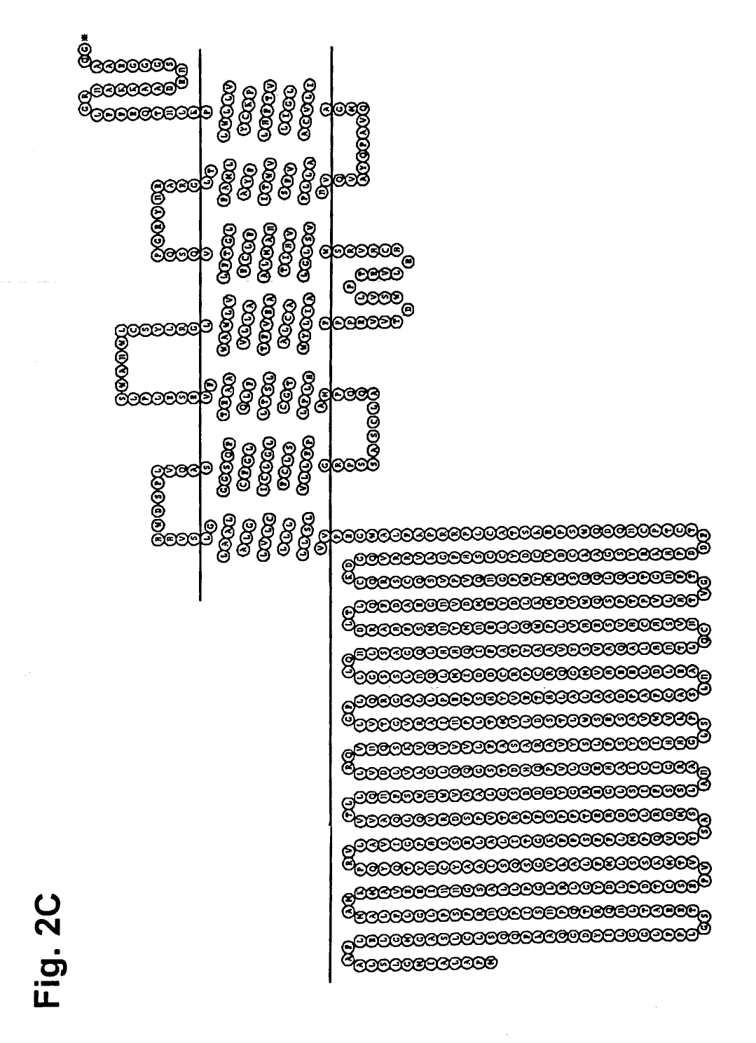

A BAC library was screened with markers within the nonrecombinant interval, and a contig was developed (FIG. 1D). A BAC clone was selected for sequencing (RPCI-23-118E21, 246 kb). Within this BAC, a gene with a 30% homology to T1R1 (a putative taste receptor) was discovered (FIG. 2A), along with other ESTs and known genes (Table 1). The human ortholog to this gene was identified from a BAC available in the public htgs database, and the predicted protein sequence was aligned with SAC1 and T1R1. SAC1 is 858 amino acids in length and contains six exons; the intron and exon boundaries were determined by sequencing of the mouse tongue cDNA (FIG. 2B). The secondary structure of this protein with regards to transmembrane domains was predicted (FIG. 2C).

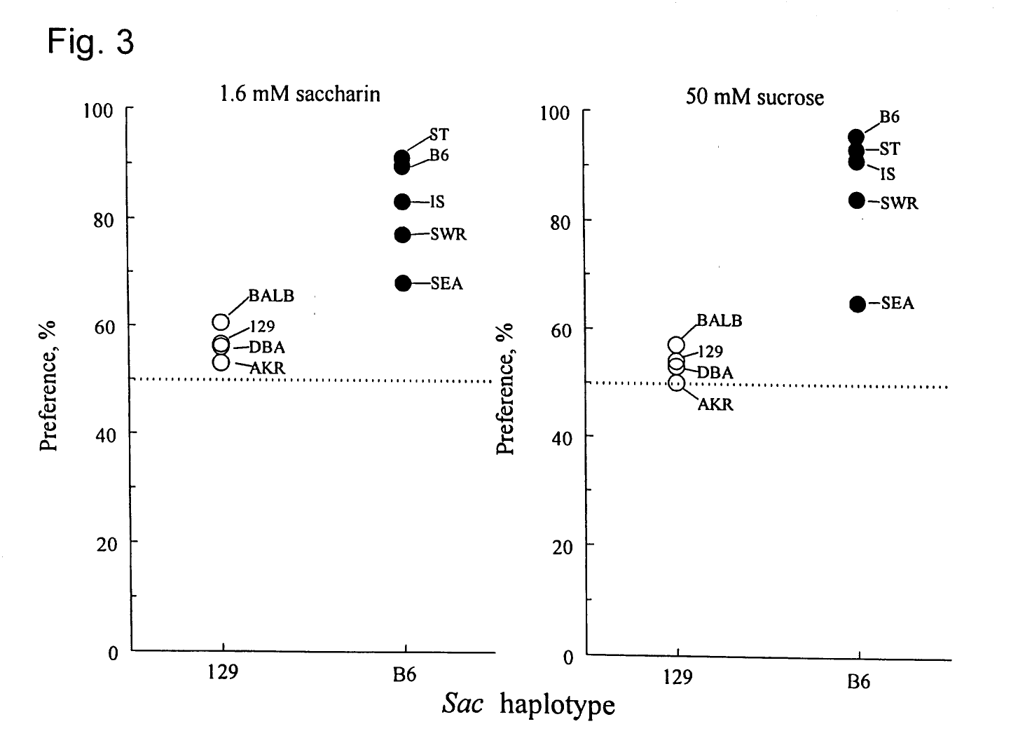

To determine whether this gene might contain functional polymorphisms that could account for the behavioral differences between the two strains, 11.8 kb of sequence, including the SAC1 gene and several kb up and downstream were amplified with PCR primers and then sequenced using DNA from the high and low preferring strains (Lush I. E., The genetics of tasting in mice. VI. Saccharin, acesulfame, dulcin and sucrose. Genet Res 1989;53:95-99; Lush I. The genetics of bitterness, sweetness, and saltiness in strains of mice. In Genetics of Perception and Communication, Vol. 3, eds. Wysocki C. and Kare M., New York: Marcel Dekker, 1991:227-235). Many variants existed between these strains, and of these, five variants were found in the low preferring strains but not in the high preferring strain. One of these variants results in a missense mutation (Ile61Thr; FIG. 2). The other four variants were in non-coding regions (T>A−2383 nt; A>G−183 nt; A>G+134 nt; T>C+651 nt, between exon 2 and 3). These five variants will be referred to as the 129-like or B6-like haplotypes. Additional inbred strains of mice with known saccharin and sucrose preferences (Lush I. E., The genetics of tasting in mice. VI. Saccharin, acesulfame, dulcin and sucrose. Genet Res, 1989;53:95-99; Lush I. The genetics of bitterness, sweetness, and saltiness in strains of mice. In Genetics of Perception and Communication, Vol. 3, eds. Wysocki C. and Kare M., New York: Marcel Dekker, 1991:227-235; Lush I. E. and Holland G. The genetics of tasting in mice. V. Glycine and cyclohexamide. Genet Res, 1988;52:207-212) were also sequenced. The 129-like haplotype was found in mice with lower sweetener preference and the B6-like haplotype was found in mice with higher sweetener preference (FIG. 3).

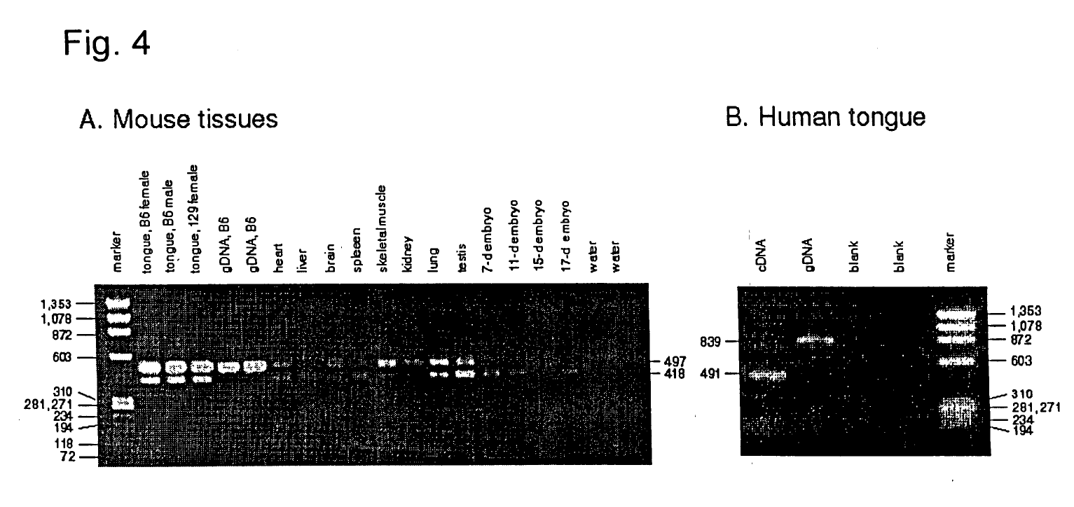

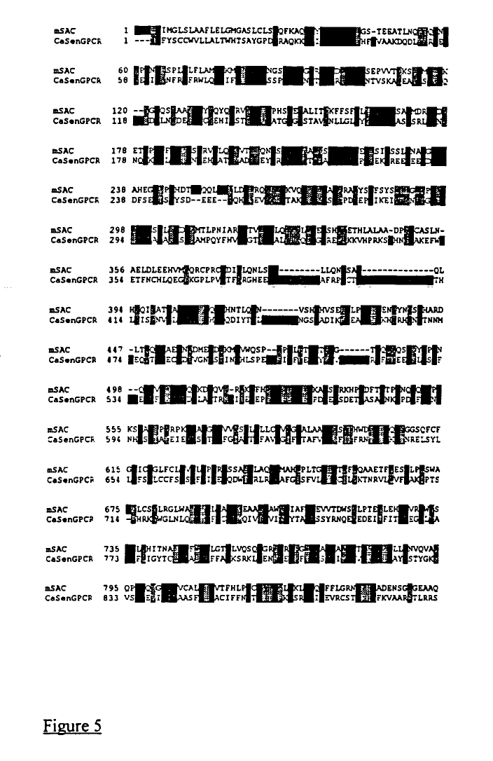

B6 mice have higher maximal gustatory neural firing in response to sweeteners compared with 129 mice, as do the 129.B6-Sac partially congenic strains (Bachmanov A. A. et al. Sucrose consumption in mice: major influence of two genetic loci affecting peripheral sensory responses. Mammalian Genome, 1997;8:545-548). Thus, the SAC1 gene is likely to be expressed in tongue. To test this hypothesis, RNA from mouse and human tongue was extracted, reversed transcribed into cDNA and primers, chosen to span an intron, were used in a PCR reaction. Genomic and cDNA yielded bands of different sizes, which were purified and sequenced (FIGS. 4AB). Sequencing results confirmed that the bands were derived from this gene with the appropriate intron/exon boundaries. Further analysis of expression in cDNA in mouse tissue, using commercially available mouse cDNA, indicated this gene is also expressed is widely expressed. The broad range of tissue expression of this gene may indicate that other tissues use this receptor to sense extra cellular sugars (FIG. 4A).

Hoon et al. identified a gene, Gpr70 (formerly TR1 or T1R1) as a putative sweet receptor based mainly on its expression in anterior tongue taste cells. Since it also mapped to distal chromosome 4, it was a logical candidate for SAC1. However, we have shown that Gpr70 is at least 4 cM proximal to SAC1 (Li X. et al. The saccharin preference locus (Sac) and the putative sweet taste receptor (Gpr70) gene have distinct locations on mouse chromosome 4. Mammalian Genome, 2001;12:13-16). Nevertheless, Gpr70 could be an additional sweet receptor and there could be others. It has been argued based upon human psychophysical studies and studies of sweet taste transduction mechanisms that there must be more than one sweet receptor. Other lines of evidence, however, are more consistent with the existence of one or a very few receptors (Bartoshuk L. M. Is sweetness unitary? An evaluation of the evidence for multiple sweeteners. In Sweetness, ed. Dobbing, J., London: Springer-Verlag, 1987:33-46). At present no evidence has been found of a family of Sac-like receptors resembling the large family of bitter receptors recently reported (Matsunami H., Montmayeur J. P., and Buck L. B. A family of candidate taste receptors in human and mouse [see comments]. Nature, 2000;404:601-604; Adler E. et al. A novel family of mammalian taste receptors [see comments]. Cell, 2000;100:693-702). The sweet substances that exist in nature, which presumably shaped the evolution of sweet receptor(s), are likely much more similar amongst themselves, mostly simple sugars, than are the vast array of structurally diverse bitter tasting compounds.

A receptor for the sugar trehalose has recently been identified in the fruit fly, Drosophila melanogaster. Surprisingly, the trehalose and other fly taste receptors, have no homology with SAC1. The specialization of flies for the sugar trehalose may account for this divergence.

There may be multiple sweet receptors; evidence from across species comparisons, psychophysical cross adaptation, and sweetness competitors has been reviewed (Bartoshuk L. M. Is sweetness unitary? An evaluation of the evidence for multiple sweeteners. In Sweetness, ed. Dobbing, J., London: Springer-Verlag, 1987:33-46). The SAC1 gene accounts for ˜40% of the genetic differences in sweet perception between these two particular strains of mice, but other receptors, and other alleles of these receptors may exist.

Because sucrose is perceived to be bad for human health, considerable resources are directed toward the discovery of high potency, low caloric sweeteners. Most of the most widely known high potency sweeteners were discovered serendipitously, i.e., the sweetener was synthesized for a different purpose and someone in the laboratory accidentally tasted it and discovered it was sweet (Walters E. D. The rational discovery of sweeteners. In Sweeteners. Discovery, molecular design, and chemoreception, eds. Walters D. E., Orthoefer F. T., and DuBois G. E., American Chemical Society, USA, 1991:1-11). More direct methods, however, have been employed to identify new sweet compounds, and the sweet receptor has been extensively modeled to predict which ligands will be sweet.

It is not known how or why different alleles of SAC1 arose in inbred strains of mice but their existence, in addition to providing us with a tool to identify a sweet receptor, raises the question of whether they might also -characterize human populations. There appear to exist reliable individual differences in human sensitivity and preference for sweet sugars but whether these are genetically influenced remains to be determined. The identification of SAC1 should facilitate research in this area. Also, the observation that SAC1 is expressed in several tissues in addition to tongue raises the interesting possibility that it could be involved in other aspects of sugar recognition and that allelic variants in this gene could be related to diseases or conditions such as diabetes and obesity.

Alleles of the gene described in this application are likely to account for the SAC1 behavioral and neurological phenotype for four reasons. First, the SAC1 nonrecombinant region is small, less than 194 kb; this gene lies within this nonrecombinant interval and the peak of LOD score corresponds closely with the location of the gene. Second, of the genes contained within this region, no others are viable candidates for SAC1. Third, this gene has sequence homology to other putative taste receptors, and is expressed in the tongue. Finally, a haplotype with a missense mutation is found in mice with low sweetener preference but not in mice with high sweetener preference. These data strongly suggest that mutations of this gene account for differences in the acceptance and preference for sweeteners attributed to the SAC1 locus.

Among the multiple mechanisms involved in regulation of ethanol intake, one of the least appreciated factors is the perception of its flavor (Nachman M., Larue C., Le Magnen J. The role of olfactory and orosensory factors in the alcohol preference of inbred strains of mice. Physiology Behavior, 1971;6:53-95). Although individual variability in the perception of ethanol flavor by adults and children was described over 60 years ago (Richter C. P. Alcohol as a food. Quart. J Studies Alcohol, 1941; 1 :650-62), the hypothesis that individual differences in alcohol chemosensory perception can affect alcohol intake did not receive due attention. As a result, the relationship between alcohol chemosensation and intake is not well-understood. Humans perceive ethanol flavor as a combination of components, including sweetness, bitterness, odor and irritation (burning sensation), which depend on ethanol concentration (Green B. G. The sensitivity of the tongue to ethanol. Ann. NY. Acad. Sci., 1987;510:315-7; Bartoshuk L. M., Conner E., Grubin D., Karrer T., Kochenbach K., Palsco M., et al. PROP supertasters and the perception of ethyl alcohol. Chem. Senses, 1993.). Rats detect sweet (sucrose-like) and bitter (quinine-like) sensory components in ethanol (Kiefer S. W., Lawrence G. J. The sweet-bitter taste of alcohol: aversion generalization to various sweet-quinine mixtures in the rat. Chem. Senses, 1988;13:633-41; Kiefer S. W., Mahadevan R. S. The taste of alcohol for rats as revealed by aversion generalization tests. Chem. Senses, 1993;18:509-22) and probably perceive the other components detected by humans as well.

The relationship between ethanol and sweetener perception and consumption has been studied the most and is supported by several lines of evidence:

(a) Electrophysiological recordings from gustatory nerves indicate that lingual application of ethanol activates sweetener-responsive neural fibers (Hellekant G., Danilova V., Roberts T., Ninomiya Y. The taste of ethanol in a primate model: I. Chorda tympani nerve response in Macaca mulatta. Alcohol, 1997;14:473-84; Sako N., Yamamoto T. Electrophysiological and behavioral studies on taste effectiveness of alcohols in rats. Am. J. Physiol., 1999;276:R388-96).

(b) Conditioned taste aversions generalize between ethanol and sucrose (Kiefer S. W., Lawrence G. J. The sweet-bitter taste of alcohol: aversion generalization to various sweet-quinine mixtures in the rat. Chem. Senses, 1988;13:633-41; Kiefer S. W., Mahadevan R. S. The taste of alcohol for rats as revealed by aversion generalization tests. Chem. Senses, 1993;18:509-22; Lawrence G. J., Kiefer S. W. Generalization of specific taste aversions to alcohol in the rat. Chem. Senses, 1987;12:591-9; Blizard D. A., McClearn G. E. Association between ethanol and sucrose intake in the laboratory mouse: exploration via congenic strains and conditioned taste aversion. Alcohol. Clin. Exp. Res., 2000;24:253-8.), suggesting that ethanol and sucrose share the same taste property, most likely sweetness.

(c) Genetic associations between preferences for ethanol and sweeteners were found among some rat and mouse strains and within their segregating crosses (Overstreet D. H., Kampov-Polevoy A. B., Rezvani A. H., Murelle L., Halikas J. A., Janowsky D. S. Saccharin intake predicts ethanol intake in genetically heterogeneous rats as well as different rat strains. Alcohol. Clin. Exp. Res., 1993;17:366-9; Sinclair J. D., Kampov-Polevoy A., Stewart R., Li T -K. Taste preferences in rat lines selected for low and high alcohol consumption. Alcohol, 1992;9:155-60; Stewart R. B., Russell R. N., Lumeng L., Li T -K., Murphy J. M. Consumptions of sweet, salty, sour, and bitter solutions by selectively bred alcohol-preferring and alcohol-nonpreferring lines of rats. Alcohol. Clin. Exp. Res., 1994;18:375-81; Belknap J. K., Crabbe J. C., Young E. R. Voluntary consumption of alcohol in 15 inbred mouse strains. Psychopharmacol., 1993;1 12:503-10; Bachmanov A. A., Reed D. R., Tordoff M. G., Price R. A., Beauchamp G. K. Intake of ethanol, sodium chloride, sucrose, citric acid, and quinine hydrochloride solutions by mice: a genetic analysis. Behav. Genet., 1996;26:563-73; Bachmanov A. A., Tordoff M. G., Beauchamp G. K. Ethanol consumption and taste preferences in C57BL/6ByJ and 129/J mice. Alcohol. Clin. Exp. Res., 1996;20:201-6), reviewed in (Kampov-Polevoy A. B., Garbutt J. C., Janowsky D. S. Association between preference for sweets and excessive alcohol intake: a review of animal and human studies. Alcohol. Alcohol., 1999;34:386-95; Overstreet D. H., Rezvani A. H., Parsian A. Behavioural features of alcohol-preferring rats: focus on inbred strains. Alcohol. Alcohol., 1999;34:378-85); with some exceptions (Phillips T. J., Crabbe J. C., Metten P., Belknap J. K. Localization of genes affecting alcohol drinking in mice. Alcohol. Clin. Exp. Res., 1994;18:931-941; Parsian A., Overstreet D. H., Rezvani A. H. Independent segregation of alcohol and saccharin intakes in F2 progeny from FH/AC1 intercross (Abstract). Alcohol. Clin. Exp. Res., 2000;24(Supplement):58A)).

(d) Human studies show that alcoholics have a stronger liking of concentrated sucrose compared with nonalcoholics (Kampov-Polevoy A. B., Garbutt J. C., Davis C. E., Janowsky D. S. Preference for higher sugar concentrations and Tridimensional Personality Questionnaire scores in alcoholic and nonalcoholic men. Alcohol. Clin. Exp. Res., 1998;22:610-4; Kampov-Polevoy A. B., Garbutt J. C., Janowsky D. Evidence of preference for a higher concentration sucrose solution in alcoholic men. American Journal of Psychiatry, 1997; 154:269-70).

There are several possible mechanisms that could underlie the association between sweetener and ethanol responses:

(a) Common peripheral taste mechanisms, which may involve the interaction of ethanol with a peripheral sweet taste transduction. At least one such common peripheral mechanism is mediated by the Gpr98 gene (SAC1 locus) encoding a sweet taste receptor (as described below).

(b) Common brain mechanisms. The regulation of ingestive responses to ethanol and sweeteners may involve common opioidergic, serotonergic and dopaminergic brain neurotransmitter systems (Gosnell B. A., Majchrzak M. J. Centrally administered opioid peptides stimulate saccharin intake in nondeprived rats. Pharm. Biochem. Behav., 1989;33:805-10; George S. R., Roldan L., Lui A., Naranjo C. A. Endogenous opioids are involved in the genetically determined high preference for ethanol consumption. Alcohol. Clin. Exp. Res., 1991;15:668-72; Hubell C. L., Marglin S. H., Spitalnic S. J., Abelson M. L., Wild K. D., Reid L. D. Opioidergic, serotoninergic, and dopaminergic manipulations and rats' intake of a sweetened alcoholic beverage. Alcohol, 1991 ;8:355-67; Pucilowski O., Rezvani A. H., Janowsky D. S. Suppression of alcohol and saccharin preference in rats by a novel Ca²⁺ channel inhibitor, Goe 5438. Psychopharmacol., 1992; 107:447-52). These mechanisms could be responsible for the emotional response to the pleasantness of ethanol or sweeteners, or the motivational mechanisms driving their intakes.

(c) Common signals related to the caloric value of ethanol and sugars (Gentry R. T., Dole V. P. Why does a sucrose choice reduce the consumption of alcohol in C57BL/6J mice? Life Sci., 1987;40:2191-4). Ethanol is metabolized in the body through some of the same pathways as carbohydrates and provides comparable energy. Thus, energy derived from carbohydrates and ethanol may have similar rewarding effects through the same hunger and satiety mechanisms.

(d) Incidental genetic linkage. Different genes affecting responses to ethanol and sweeteners may reside nearby on the same chromosome.

Ethanol consumption is a complex trait, depending on multiple mechanisms of its regulation and determined by multiple genes. A body of evidence suggests that ethanol consumption may depend on perception of its flavor, and that there is an association between perception and consumption of ethanol and sweet-tasting compounds. However, only a few genes have been identified as candidates affecting ethanol consumption.

The present invention provides that a gene, SAC1, is associated with the detection of a sensing of carbohydrates, other sweet compounds, and alcohols including ethanol. The sequence of the mouse SAC1 cDNA (SEQ ID NO: 1) is:

ATGCCAGCTTTGGCTATCATGGGTCTCAGCCTGGCTGCTTTCCTGGAGCT
TGGGATGGGGGCCTCTTTGTGTCTGTCACAGCAATTCAAGGCACAAGGGG
ACTACATACTGGGCGGGCTATTTCCCCTGGGCTCAACCGAGGAGGCCACT
CTCAACCAGAGAACACAACCCAACAGCATCCCGTGCAACAGGTTCTCACC
CCTTGGTTTGTTCCTGGCCATGGCTATGAAGATGGCTGTGGAGGAGATCA
ACAATGGATCTGCCTTGCTCCCTGGGCTGCGGCTGGGCTATGACCTATTT
GACACATGCTCCGAGCCAGTGGTCACCATGAAATCCAGTCTCATGTTCCT
GGCCAAGGTGGGCAGTCAAAGCATTGCTGCCTACTGCAACTACACACAGT
ACCAACCCCGTGTGCTGGCTGTCATCGGCCCCCACTCATCAGAGCTTGCC
CTCATTACAGGCAAGTTCTTCAGCTTCTTCCTCATGCCACAGGTCAGCTA
TAGTGCCAGCATGGATCGGCTAAGTGACCGGGAAACGTTTCCATCCTTCT
TCCGCACAGTGCCCAGTGACCGGGTGCAGCTGCAGGCAGTTGTGACTCTG
TTGCAGAACTTCAGCTGGAACTGGGTGGCCGCCTTAGGGAGTGATGATGA
CTATGGCCGGGAAGGTCTGAGCATCTTTTCTAGTCTGGCCAATGCACGAG
GTATCTGCATCGCACATGAGGGCCTGGTGCCACAACATGACACTAGTGGC
CAACAGTTGGGCAAGGTGCTGGATGTACTACGCCAAGTGAACCAAAGTAA
AGTACAAGTGGTGGTGCTGTTTGCCTCTGCCCGTGCTGTCTACTCCCTTT
TTAGTTACAGCATCCATCATGGCCTCTCACCCAAGGTATGGGTGGCCAGT
GAGTCTTGGCTGACATCTGACCTGGTCATGACACTTCCCAATATTGCCCG
TGTGGGCACTGTGCTTGGGTTTTTGCAGCGGGGTGCCCTACTGCCTGAAT
TTTCCCATTATGTGGAGACTCACCTTGCCCTGGCCGCTGACCCAGCATTC
TGTGCCTCACTGAATGCGGAGTTGGATCTGGAGGAACATGTGATGGGGCA
ACGCTGTCCACGGTGTGACGACATCATGCTGCAGAACCTATCATCTGGGC
TGTTGCAGAACCTATCAGCTGGGCAATTGCACCACCAAATATTTGCAACC
TATGCAGCTGTGTACAGTGTGGCTCAAGCCCTTCACAACACCCTACAGTG
CAATGTCTCACATTGCCACGTATCAGAACATGTTCTACCCTGGCAGCTCC
TGGAGAACATGTACAATATGAGTTTCCATGCTCGAGACTTGACACTACAG
TTTGATGCTGAAGGGAATGTAGACATGGAATATGACCTGAAGATGTGGGT
GTGGCAGAGCCCTACACCTGTATTACATACTGTGGGCACCTTCAACGGCA
CCCTTCAGCTGCAGCAGTCTAAAATGTACTGGCCAGGCAACCAGGTGCCA
GTCTCCCAGTGTTCCCGCCAGTGCAAAGATGGCCAGGTTCGCCGAGTAAA
GGGCTTTCATTCCTGCTGCTATGACTGCGTGGACTGCAAGGCGGGCAGCT
ACCGGAAGCATCCAGATGACTTCACCTGTACTCCATGTAACCAGGACCAG
TGGTCCCCAGAGAAAAGCACAGCCTGCTTACCTCGCAGGCCCAAGTTTCT
GGCTTGGGGGGAGCCAGTTGTGCTGTCACTCCTCCTGCTGCTTTGCCTGG
TGCTGGGTCTAGCACTGGCTGCTCTGGGGCTCTCTGTCCACCACTGGGAC
AGCCCTCTTGTCCAGGCCTCAGGTGGCTCACAGTTCTGCTTTGGCCTGAT
CTGCCTAGGCCTCTTCTGCCTCAGTGTCCTTCTGTTCCCAGGGCGGCCAA
GCTCTGCCAGCTGCCTTGCACAACAACCAATGGCTCACCTCCCTCTCACA
GGCTGCCTGAGCACACTCTTCCTGCAAGCAGCTGAGACCTTTGTGGAGTC
TGAGCTGCCACTGAGCTGGGCAAACTGGCTATGCAGCTACCTTCGGGGAC
TCTGGGCCTGGCTAGTGGTACTGTTGGCCACTTTTGTGGAGGCAGCACTA
TGTGCCTGGTATTTGATCGCTTTCCCACCAGAGGTGGTGACAGACTGGTC
AGTGCTGCCCACAGAGGTACTGGAGCACTGCCACGTGCGTTCCTGGGTCA
GCCTGGGCTTGGTGCACATCACCAATGCAATGTTAGCTTTCCTCTGCTTT
CTGGGCACTTTCCTGGTACAGAGCCAGCCTGGCCGCTACAACCGTGCCCG
TGGTCTCACCTTCGCCATGCTAGCTTATTTCATCACCTGGGTCTCTTTTG
TGCCCCTCCTGGCCAATGTGCAGGTGGCCTACCAGCCAGCTGTGCAGATG
GGTGCTATCCTAGTCTGTGCCCTGGGCATCCTGGTCACCTTCCACCTGCC
CAAGTGCTATGTGCTTCTTTGGCTGCCAAAGCTCAACACCCAGGAGTTCT
TCCTGGGAAGGAATGCCAAGAAAGCAGCAGATGAGAACAGTGGCGGTGGT
GAGGCAGCTCAGGGACACAATGAATGA

The geonomic DNA sequence of the mouse SAC1 gene (SEQ ID NO: 2) is:

ATCTGAGCCTTAGACACAGCACTGGTGCCAGGCAAACACTCCTGGGCCTA
CATGCTTGGG
GCCTCTTCATATTCCAAAAGCTGTCTTTGGGTAAGATGAAGTTCCTCTGG
CAGTGGCATG
AGTGCTGAAGGCTCTTTCCCTGCCCTTCACCTGCTTTCTTGATAGTCTCT
CTGCATACCA
AACAGGCCCTTGTCTCCTGGGAAATGGAAACTATGAAATCAATAGCTGAG
GCTTCTCTAG
GAAAGCCTGCCCTGGTCAGTACAACCTGTTTCACAGCTTCTATAGAATAG
TTACATCAGC
CTTCTGAAGATGGCCTCTTAGAGCACATGCACCCCCAAGATTCTAAGATG
TCAATACTAA
CTGACCAAACCATACCTCTCTAGCCAGCCCTGCTGCTCCTGTTGTCTGGT
ACCCAGGTGA
CTGAGGACATGACTGGTGGAAGGAAACTAGGCCCCTTTGTCTGTCAGATG
GCCATACCCA
GCATGGCTGATGCCCAGTGTATAAGACCCTACGCTTTTCCACTGGTCTTA
ATGTTAAACC
CTAGGACAGTGTCCTCAGCATAGCTGGTGTGTGTGAATGCAAACTTTGGG
GCATATCTCT
TCCATTAAGCACTGTGATATATGTAGTATTTCCAACAAATAAATTATACC
TACATGATTG
GGTATAGCATTCTGGGATGGGTCACAGGTGTGTCAGGTGCCTAATTATGT
GGGGGAAGAA
CATAGAAATATATAGGTGGGGAGGGAGCTAACCCTAGGAATAAGGCTAAA
GCATGTGTCT
CCAGTCCTGAAGACTCAAAGGGCAACGTGAATCATGAGACATGTTCAGGA
CTGAAGGAGT
TGCCATGTATCTGTCCTTGATGTATCTTAATCATACATACACTATGAGAT
CTGTGTTACC
TCCATTTTGCAGGTGAGAAAAGAAACACCTGAATGGCCTACCTTAAAGGG
CTAAGTGGGA
AAATAGGTCTGAAGATAACCCAGGCACTGTGTGACAAAGCGGGAAGAAAA
CTAGAGATGC
TTTCTTCATGGCAACAACCTAGAGGGTACAACCTAGTGGTTTCTTCTTGG
TACTCCACTG
TATACACCCCATCTGCTTGGGCTGTACATTGTCTGACCATGCTTATAACA
AAAGTCACAT
ACTACTAGCCAAGACTGAGAACTTAGAGCGACTGGCCAGAAAGTAAAGAT
ACAACAGTTG
ATATGTGTGCCACACACAGATCCATGTGTACATGTCTATTAATTATGTGA
ACGTGCTTTG
TGGACATCCTCACAAAGCAGCAGGGAAATGCAAAGGTCATTTCCATAACA
CCTGCTGGAC
ACCATATGACATTGAGATTACCGGGGTGCCCATTCCAACAAGAGTTAATA
GCTCCCCCTA
TGTTTGGGTGCCAGAAACCTGATTTGTTAGCAATAGCTCCCTCACATCCA
GATTAAGAGG
GGGATGGCTTAGCTAGGGTTACTATGATGAAACTATGACCAAAGCAACTT
GTGGGTAAAA
GGGTGTATTTGGCTTACACTTCCATATCACTTCATCAAAGTGAGGACAGG
AACTCAAATA
GAGTAGGAATTTGGTGACAAGAGCTGATGTAGAGGCAATGCAGTGGTGCC
ACTTAGTGGC
GCGCTCAGTCTGCTCCCTTTCTTAATAGAATGCAAGACCACCAGCCCATG
GGTGGCACCA
CAATGGGACCGGGCCCTTCCCCATCGGTCACTAAGAAAATGCCCTACAGC
CAGATCTTAT
GGAGACATTTTCTCAACGGAGGCTCACTCCTTTCAGATAACTCTATATCA
AATTGACATA
AACCAGAACAGAGGAGGAGGCTAAGAAGGAAACTGCCAATTGCATACATG
CACACACCTG
GCCCTAGCAGCTGCAGGAAGCTATTTGTTTATGGCCTTTTCTCATTTTCA
TGGACCAGCA
TGAGCACTCTGCAGAGAGAGATGCCTGCATGCCTGCCAAGGCAGGAGTGC
TTACACTGAA
GGTCAACAGGATGGCAGGGGGGCTGCAGAGCTTCCAAGTGTCAGAACCCC
AGCAGAAGAG
CTGAGACCCTTGCCCGAGGACTCAGGCGGGTTGGGAAGGCCAGGAAATTC
AGCCAGAGCT
CTTCTTCAGATGGGGTACCATCTGAAGGTTAGACCAGCTAGCCAGCTGTT
GTTGAGGGAC
CACCTCTGCAGCCCCTACCTTTGGAAGATAGAAAGTGTCTCTGTGACAAG
TATGGCCATT
GTGCCCCCTTATTCCACAGTCAACAGAAACCCTGGAATCCTGAACACTTC
TGCAGCTTCT
TTTTTACAGTCTGCCAGGTTGCTCTAGGAATGAAGGGTGCCGAGAGGCTT
GGGCGTAGGC
AGGTGACAAGACCACAGTTAGTGGTCACAGCTGGCTTACTGGATCACTCT
TGGACAGAGT
TTGTTAGATATGGAGTGGAGTATACACAAGGCATCAGGCGGGGGATATTG
AATGTATCAC
CGGAGCTCCTTGGGGCTTGGCAGCCAAGCACAGCAGTGGTTTTGCTAAAC
AAATCCACGG
TTCCCTCCCCTTGACGCAGTACATCTGTGGCTCCAACCCCACACACCCAC
CCATTGTTAG
TGCTGGAGACTTCTACCTACCATGCCAGCTTTGGCTATCATGGGTCTCAG
CCTGGCTGCT
TTCCTGGAGCTTGGGATGGGGGCCTCTTTGTGTCTGTCACAGCAATTCAA
GGCACAAGGG
GACTACATACTGGGCGGGCTATTTCCCCTGGGCTCAACCGAGGAGGCCAC
TCTCAACCAG
AGAACACAACCCAACAGCATCCCGTGCAACAGGTATGGAGGCTAGTAGCT
GGGGTGGGAG
TGAACCGAAGCTTGGCAGCTTTGGCTCCGTGGTACTACCAATCTGGGAAG
AGGTGGTGAT
CAGTTTCCATGTGGCCTCAGGTTCTCACCCCTTGGTTTGTTCCTGGCCAT
GGCTATGAAG
ATGGCTGTGGAGGAGATCAACAATGGATCTGCCTTGCTCCCTGGGCTGCG
GCTGGGCTAT
GACCTATTTGACACATGCTCCGAGCCAGTGGTCACCATGAAATCCAGTCT
CATGTTCCTG
GCCAAGGTGGGCAGTCAAAGCATTGCTGCCTACTGCAACTACACACAGTA
CCAACCCCGT
GTGCTGGCTGTCATCGGCCCCCACTCATCAGAGCTTGCCCTCATTACAGG
CAAGTTCTTC
AGCTTCTTCCTCATGCCACAGGTGAGCCCACTTCCTTTGTGTTCTCAACC
GATTGCACCC
ATTGAGCTCTCATATCAGAAAGTGCTTCTTGATCACCACAGGTCAGCTAT
AGTGCCAGCA
TGGATCGGCTAAGTGACCGGGAAACGTTTCCATCCTTCTTCCGCACAGTG
CCCAGTGACC
GGGTGCAGCTGCAGGCAGTTGTGACTCTGTTGCAGAACTTCAGCTGGAAC
TGGGTGGCCG
CCTTAGGGAGTGATGATGACTATGGCCGGGAAGGTCTGAGCATCTTTTCT
AGTCTGGCCA
ATGCACGAGGTATCTGCATCGCACATGAGGGCCTGGTGCCACAACATGAC
ACTAGTGGCC
AACAGTTGGGCAAGGTGCTGGATGTACTACGCCAAGTGAACCAAAGTAAA
GTACAAGTGG
TGGTGCTGTTTGCCTCTGCCCGTGCTGTCTACTCCCTTTTTAGTTACAGC
ATCCATCATG
GCCTCTCACCCAAGGTATGGGTGGCCAGTGAGTCTTGGCTGACATCTGAC
CTGGTCATGA
CACTTCCCAATATTGCCCGTGTGGGCACTGTGCTTGGGTTTTTGCAGCGG
GGTGCCCTAC
TGCCTGAATTTTCCCATTATGTGGAGACTCACCTTGCCCTGGCCGCTGAC
CCAGCATTCT
GTGCCTCACTGAATGCGGAGTTGGATCTGGAGGAACATGTGATGGGGCAA
CGCTGTCCAC
GGTGTGACGACATCATGCTGCAGAACCTATCATCTGGGCTGTTGCAGAAC
CTATCAGCTG
GGCAATTGCACCACCAAATATTTGCAACCTATGCAGCTGTGTACAGTGTG
GCTCAAGCCC
TTCACAACACCCTACAGTGCAATGTCTCACATTGCCACGTATCAGAACAT
GTTCTACCCT
GGCAGGTAAGGGTAGGGTTTTTTGCTGGGTTTTGCCTGCTCCTGCAGGAA
CACTGAACCA
GGCAGAGCCAAATCTTGTTGTGACTGGAGAGGCCTTACCCTGACTCCACT
CCACAGCTCC
TGGAGAACATGTACAATATGAGTTTCCATGCTCGAGACTTGACACTACAG
TTTGATGCTG
AAGGGAATGTAGACATGGAATATGACCTGAAGATGTGGGTGTGGCAGAGC
CCTACACCTG
TATTACATACTGTGGGCACCTTCAACGGCACCCTTCAGCTGCAGCAGTCT
AAAATGTACT
GGCCAGGCAACCAGGTAAGGACAAGACAGGCAAAAAGGATGGTGGGTAGA
AGCTTGTCGG
TCTTGGGCCAGTGCTAGCCAAGGGGAGGCCTAACCCAAGGCTCCATGTAC
AGGTCCCAGT
CTCCCAGTGTTCCCGCCAGTGCAAAGATGGCCAGGTTCGCCGAGTAAAGG
GCTTTCATTC
CTGCTGCTATGACTGCGTGGACTGCAAGGCGGGCAGCTACCGGAAGCATC
CAGGTGAACC
GTCTTCCCTAGACAGTCTGCACAGCCGGGCTAGGGGGCAGAAGCATTCAA
GTCTGGCAAG
CGCCCTCCCGCGGGGCTAATGTGGAGACAGTTACTGTGGGGGCTGGCTGG
GGAGGTCGGT
CTCCCATCAGCAGACCCCACATTACTTTTCTTCCTTCCATCACTACAGAT
GACTTCACCT
GTACTCCATGTAACCAGGACCAGTGGTCCCCAGAGAAAAGCACAGCCTGC
TTACCTCGCA
GGCCCAAGTTTCTGGCTTGGGGGGAGCCAGTTGTGCTGTCACTCCTCCTG
CTGCTTTGCC
TGGTGCTGGGTCTAGCACTGGCTGCTCTGGGGCTCTCTGTCCACCACTGG
GACAGCCCTC
TTGTCCAGGCCTCAGGTGGCTCACAGTTCTGCTTTGGCCTGATCTGCCTA
GGCCTCTTCT
GCCTCAGTGTCCTTCTGTTCCCAGGGCGGCCAAGCTCTGCCAGCTGCCTT
GCACAACAAC
CAATGGCTCACCTCCCTCTCACAGGCTGCCTGAGCACACTCTTCCTGCAA
GCAGCTGAGA
CCTTTGTGGAGTCTGAGCTGCCACTGAGCTGGGCAAACTGGCTATGCAGC
TACCTTCGGG
GACTCTGGGCCTGGCTAGTGGTACTGTTGGCCACTTTTGTGGAGGCAGCA
CTATGTGCCT
GGTATTTGATCGCTTTCCCACCAGAGGTGGTGACAGACTGGTCAGTGCTG
CCCACAGAGG
TACTGGAGCACTGCCACGTGCGTTCCTGGGTCAGCCTGGGCTTGGTGCAC
ATCACCAATG
CAATGTTAGCTTTCCTCTGCTTTCTGGGCACTTTCCTGGTACAGAGCCAG
CCTGGCCGCT
ACAACCGTGCCCGTGGTCTCACCTTCGCCATGCTAGCTTATTTCATCACC
TGGGTCTCTT
TTGTGCCCCTCCTGGCCAATGTGCAGGTGGCCTACCAGCCAGCTGTGCAG
ATGGGTGCTA
TCCTAGTCTGTGCCCTGGGCATCCTGGTCACCTTCCACCTGCCCAAGTGC
TATGTGCTTC
TTTGGCTGCCAAAGCTCAACACCCAGGAGTTCTTCCTGGGAAGGAATGCC
AAGAAAGCAG
CAGATGAGAACAGTGGCGGTGGTGAGGCAGCTCAGGGACACAATGAATGA
CCACTGACCC
GTGACCTTCCCTTTAGGGAACCTAGCCCTACCAGAAATCTCCTAAGCCAA
CAAGCCCCGA
ATAGTACCTCAGCCTGAGACGTGAGACACTTAACTATAGACTTGGACTCC
ACTGACCTTA
GCCTCACAGTGACCCCTTCCCCAAACCCCCAAGGCCTGCAGTGCACAAGA
TGGACCCTAT
GAGCCCACCTATCCTTTCAAAGCAAGATTATCCTTGATCCTATTATGCCC
ACCTAAGGCC
TGCCCAGGTGACCCACAAAAGGTTCTTTGGGACTTCATAGCCATACTTTG
AATTCAGAAA
TTCCCCAGGCAGACCATGGGAGACCAGAAGGTACTGCTTGCCTGAACATG
CCCAGCCCTG
AGCCCTCACTCAGCACCCTGTCCAGGCGTCCCAGGAATAGAAGGCTGGGC
ATGTATGTGT
GTGTGTGTGTGTGTGTGTGTGTGTGTGTGTGTGTGTGTATGTACGTATGT
ATGTATGTAT
CAGGACAGAACAAGAAAGACATCAGGCAGAGGACACTCAGGAGGTAGGCA
ACATCCAGCC
TTCTCCATCCCTAGCTGAGCCCTAGCCTGTAGGAGAGAACCAGGTCGCCG
CCAGCACCTT
GGACAGATCACACACAGGGTGCGGGTCAGCACCACGGCCAGCGCCAGCCA
CGCGGGACCC
CTGGAATCAGCTTCTAGTACCAAGGACAGAAAAGTTGCCGCAAGGCCCCT
TACTGGCCAG
CACCAGGGACAGAGCCACATGCCTAAGCGGCAAGGGACAAGAGCATCGTC
CATCTGCAGG
CAGGATCAGACCCGGGTCAGTTCTGGACTGGCCCCCACACCTGAATCCCG
GAGCAGCTCA
GCTGGAGAAAAGAGAAACAAGCCACACATCAGTCCCATAAAATTAAACGC
TTTTTTTAGT
GTTTAAAATAGCATTTACACAGAAGCAGCATTTACACAGAAGCAGCTCTA
TGTCAACTAC
CCAGTCACTCAGACTTTGACACAGTGTCTAGTGTAGATGTGTGGGGCCGC
TGTGCCGGGA
TGGCAGTGGCACATGATGATGGGCAGCCACCAGAACAGAAACAGAACAGG
GCCCAGCTCT
GCAGCTCTTGTGTTCACTGTCACCCACCACTGAGACTGAGACAGTGGCTA
GGTGCCAGGT
CTCTCTCCTGTCTCTCCTACTAGCTACCCTTCACATACCTTCAGTACAAA
CTGTGTTGTC
ATGTGCCAAGTAGCAGGTGGGGAAAGGGGCATGCAAACTGCCCCTTTGGG
TAACTAGCTG
CCACCCTTAGAGCAGGCAGGCTAGCAATAAATAAATAAGTTAGACCCCAC
CTGGGCAGCC
AGAGAGGTTTGAAGGCTCTGTCTAACCCCTCAAAAATCCCACCTTGGCCT
GACAGGTGAG
GCCCATGAACTTAGCGACAGTCAGCCTGTGTCCCTGTGCACAGTTCTGTG
AGGCTTTGGG
GCAAGGGGTACCAAGAGCCCAAGAGAGCCTTTCTTGTTCTAAATGGAGGT
CACTTCCAAA
GAAGGGAACCAGGAGGTGGTCCCTGAGACTTGTGCTGAGGACTTAAAGTC
AGAGATGTCT
CCTTACAAGACTCTATAGATACTTGAGCTGTACCACCATCAGCAGCCCCA
AGAGCAGACA
AAATGTCAAGCCAATATCCTGGTGGTATGGCTGCCCTCAGGCCCTCCTCT
GTAGCCTGCT
CCCTCTGCCCTGGCCCAGAGCCCACAGCTGATCTATCCTGGCTGGCCACC
ACCACGGCCA
GCGCAGAGCTCCTGGCACAGCAGGAGCACAGACTCAGCCACAGGCAGCGC
TGAAGACATT
GGTTGATCATCACATGATGTCCACAAAGAACTCACAGGGGTTTCCCATGG
CCTTTTGGAA
GGACTGGCGGCTACCTGTAAGTTCTGGAGGGACAGCAGCCAGCTCCCGGA
CGGGTGGCCC
TCCAGGTGGCCCACCCACTACTGCATAGGCCTTTGTAAGGGGGTGCAGTG
GGGGGAGCCC
TGGGGCAACAGCTGAAGCCTGACTTCGAGGGCTACTGCCACGGCTAAGCT
GGCTGACAGG
CCGCTCCCACCAGCCGGTGCTACCAGACCCACTTGGTACTGTGTGGTCTG
ATTCACTGCC
ACTACCCCCAGCTCCAGTTGCCCGGCGCTCCTCTCGGCCTGGGGTCCGAT
GGCTGCTCCG
TGTGGACCCACTGCTCTTGCTCCCTAGGGGGAGGGAAGGGGACAACAGAG
TCAGCACGAG
GCCTGGCCACTTCCAGGGCCACCAGCTGCTCCCAGACAGTCAGGGCAGGA
CCTGGTAAGC
CTGGAGATGGTAGGGGAATGGCAGCCATGCAGATACCAGGAACAGCTGAG
AGGCGAGAAG
CTAGGGGCAGTGGCAGACAGCAGGGACAACAGGGGCCAGCCTGGCACCCC
ACACCTAACC
CCAATGCTTGAACCAAGGGTTAATGTTACAGCTGAGAAACTAAAAACCAG
CGAAGGCCCT
GTGTGCCCAGCATTCCCATTAGCCATCCTGGGTTCACCACCCAAAGACCC
AACCAGGGTC
CACCCAACCCCAGGACCCTGGTCATCTAATTTGCTTAGCCCCTGTCCTGA
AAGTAGTGGG
AACCTGAAAACACGTGCTGGCTGGGGACATGCTGAGAGGGACACAGGGGG
ACCTGGCTTA
CCGGCCCGAGAGTCCACTCTGCTAGTCCTTCAGTCTAAGGCTTGCTCAGC
ACAAAGCAAG
GGATAGCACAAGTCACACACCAGTCCAGTGCTCACCAATGGCTAATAGGA
CGATTTTGGG
CCAAGCTGAGCCTGGGTACATGCAAGGGCCTGTCCATGGTCAGGATTCAC
TCGATAGCTT
CCCCTTGGGCTTTGCCACCCTCTGGCCCAACCTCTCCTGAGTCTTTCTCT
GGACCTTGTA
GCACAAGTGTGCCCCACTCTGCCTAAGACCTCCACATCAGTCCATCTCCT
CCTGAGGGAC
ACCCACCCTTCAAGATCTTCAATATCCCTGGGATATGCTTTAACACTGAT
ATGCTTTAAC
AGTGTTGCTTGATACTCTTATCTGGCACTCTGTTGGGATGCAGGCTCCAT
AACTGATAAA
GCCCATTCTCCCCCTAGCTTGGGGCCTAGAGAGTGCCCCTACCTGCTATC
AGTGGTTACT
TTCATTCTTGCCATATCATCTCCTGGCCTCTTGCCTCTGCCACCTAGCAC
ACCAGGCTGT
CTTCCTATTCTCTAACGGCTTCTACCCACATCAGCCCCTCCCTGTCCCAC
ACACTGACTC
TTGAGATGGAACCCACCGGGACTCAAACACACAGCAGGAGCACAGAGGGA
AGCGTCGGGG
CCAGGCAGAGCGTGGGAGTGGGAGGGAGTGGGAGGAGGGGTGGCACGCCT
CTCACCTTCA
CTCTGCTGGCTCCCAGCACTGCCGCTGCCGCAGCTGAAGCCAGGGTCCTG
GTAAGCAGGC
GGGAAGCAGGGCGGGGGTCCTGGGTACTGGTAGGGGTAGCCTTGACCCAA
GGGCCAGGGT
ACTGATGGGTGGGGCAGTGGGGCCAGTGTGTCCTGATCTGAGGCTCCACT
GGAGCCACTG
TTGAGGTTCAGGGATGCGAGGTCTGGCAGGGAGGGAGGGAGGGAGGGGTA
AGTGAAGGCA
AATGAATGAGGCCACAGCAACCCTACCCAACCGCACCCCTACTCACTACT
GCACAGGTCG
CCAAAGACATAGTAGCACTGCTCAGAAAAGGTGATCTTGTTCACGGTGTG
CCTCAGGAAA
CCGTGCTTCAGCATACTGCTGGCATACTTTCTTGCCTCCCTTCGCTCCTT
GAAGCCCTCC
ACGTGTGTGTACAGCCAGTCCACCACATCCGCCCCTGGCCACAGGTCCAT
CAAAGTCAGG
GTAGCTGAGCCCTGGGAAGCTACGCCAGAATGAGGAACAGACGGGGCCCT
TCCCACACAG
CCAGGGACTCACCAATGACAGCATTGGCAATGGTGATCTTAAGCCACATG
CGGTCCCGGA
TCTCCAGTCCTGAGTCTGGCAACTGCATGACGCGGACAATGGCACTCATG
TCACTCTTCA
CAGTCAGCGGTGCCTCCTCAAGCTCTGCAGAGCACACTTCCCTGAGCCCA
GGCTCACAGC
GTGAACCTCCATGGGGTTGAGAGCAGGGGCCAGGGTCAAACCTCTTATCT
CCCATCCTTG
GGAGATGCCCCTCATCGAAACTTGAGCTAAGACCGGGAGATTCTTCCCCG
TCCCACAGTG
CAAGTCCACGTAGGCAAGGCAGCCCCCCTCCCCTCCCCGGAGAGAACAAG
CTGTTAGCTA
TGTTAGGTAGCAGAAAAGCAAAGCAGAGGCTGCCATGTCCTCCCAATTCC
CCCCTCCGCA
CAGGCCTGGCAGGACCCTCAATTCATGCAGATGACCAGTATGGCCAGGCC
TGGAGGGATA
TGTACATGTATCTTTGTGTACACATTTGTGAAGGTGTTGGAAGCAAACAA
AACCTTCATA
TGTAATGGGCCCCTGTAATAGCTCTGATGAGCACCAAAGCTCAAAGCTAG
AACTGACCAT
TGTCCTTCAACCTCAGTTTCCTTGGGTGGGGGGGGGTCCTGTGAGCTGCC
ACTTACGTGG
GGCGCCAGGCACTGAGCTGGTTAGTGAGGAAGAGCTGGTGCGTGTGATGG
CGCTGGAGCA
GGGACTCGTACCATAGCGGGGCAGGGCACCCGTCAGTGCTGCTGTGTGGG
ACAGCCAGGC
AGCCGGGTCGATGGGTCGCACTGGGTCAGCTGCATAGTTTCCACAGCAAC
GGATTACAGG
TGGTAAGTAGGGGGGCAGCACAGAGGCAGACAAGAAAGACCCCCAGACTG
AACACAGAAA
CCCCACCCTACCCCACCTTTCCATGGGGTAACTCACCCCTTGGGATGGTG
AAGTAGCTCC
GAGGGGTTGGGTCCCAGCACTTGGCCACTGTGAGACTGATGGGCCTACAG
AGTTGAGCAG
ACCATGTTGTAAGTGAGGCCCGCACAGCCCCTCCCATCCTGTGCCACTCC
CACCCCCACT
TGGCTCCCACCTCACCCTGTCTGGGACACGATCTCCCGAAGCACCCGTAC
AGCGTCGTCA
TTGCTCATGTTCTCAAAGTTGACATCGTTCACCTACGGGGTTTGTGGGGT
CAGGGGTTGG
TGGTGGGATGTGGGTGCCTCTTGTCCCCACAGTCCCCACATGGCTCCCAC
CTGCAGCAAC
ATGTCGCCCGGCTCAATGCGGCCATCAGCAGCCACGGCCCCGCCCTTCAT
GATGGATCCA
ATGTAGATGCCGCCATCACCCCGGTCGTTGCTCTGGCCCACGATGCTGAT
GCCCAGGAAG
TGGTGCCTCTCTGCAGGAGGGGCCGTGAGCAGGCCCCCAAAGCTCCCGAG
GCTGTACCCA
CCCCCAGCAGGCACCCACAGCCCACAAGGCCTCACCCATGTTGAGAGTGA
CGGTGATGAT
GTTCAGGGACATGGTGGAGTCTGTGATGCTGCTGAAGGAGGATGCCTGCG
GAGGGACCCA
GTGAGGGGCTGTGTGGGCACCATTCAGAGCAGACACCCCACCCACCTGCT
GCCTACCCGG
TCTGTCTGCCTCAAGCGCTGCTTCCGACGACGGCATTTGTGCTTCCGAAC
TAGCCGAGAG
GAGGTGCTCTGCTCTGTGGAGCTGCTCAGCCTGAGGCAGGAGTCAGAAAA
GCACAAACAT
GTATAACCAGCTCGGACGCTCAACTACAAATCTCCAGCACGTACTGACAT
GTGCACACGT
CACCCACCGGCTCGTATTGTCCTCCTCATCTGAGTCAATAAAGCTGCTAG
ATTCAAGCTC
ACTGCTCAGTACAGTGGATGCACTGTCTGGAGGTAGTCCCAGGTCCCGCC
GCCGATCCCC
TCTCGGGTGCCCATTGGTCCGGGCAGCTGTGGGGACAGTAGGGTGGGTAC
GACTGTGGGA
CTTCAGTCCTAACAGAATGCGGGTGGCCTGTGCATTTCAAAGTTTATGCA
GTAACTCTGG
GGCCACAGGGGCTAGGAGTACCAGGCTGGGACCTCTACCCAAGGATCACT
GCTTGGAAGA
ATATGTGGAATACTTCCAGGCTTGGAGTATACCAAAGGGATACCAAGGG

The polypeptide sequence of mouse SAC1 (SEQ ID NO: 3) is:

MPALAIMGLSLAAFLELGMGASLCLSQQFKAQGDYILGGLFPLGSTEEAT
LNQRTQPNSIPCNRFSPLGLFLAMAMKMAVEETNNGSALLPGLRLGYDLF
DTCSEPVVTMKSSLMFLAKVGSQSIAAYCNYTQYQPRVLAVIGPHSSELA
LITGKFFSFFLMPQVSYSASMDRLSDRETFPSFFRTVPSDRVQLQAVVTL
LQNFSWNWVAALGSDDDYGREGLSIFSSLANARGICIAHEGLVPQHDTSG
QQLGKVLDVLRQVNQSKVQVVVLFASARAVYSLFSYSIHHGLSPKVWVAS
ESWLTSDLVMTLPNIARVGTVLGFLQRGALLPEFSHYVETHLALAADPAF
CASLNAELDLEEHVMGQRCPRCDDIMLQNLSSGLLQNLSAGQLHHQIFAT
YAAVYSVAQALHNTLQCNVSHCHVSEHVLPWQLLENMYNMSFHARDLTLQ
FDAEGNVDMEYDLKMWVWQSPTPVLHTVGTFNGTLQLQQSKMYWPGNQVP
VSQCSRQCKDGQVRRVKGFHSCCYDCVDCKAGSYRKHPDDFTCTPCNQDQ
WSPEKSTACLPRRPKFLAWGEPVVLSLLLLLCLVLGLALAALGLSVHHWD
SPLVQASGGSQFCFGLICLGLFCLSVLLFPGRPSSASCLAQQPMAHLPLT
GCLSTLFLQAAETFVESELPLSWANWLCSYLRGLWAWLVVLLATFVEAAL
CAWYLTAFPPEVVTDWSVLPTEVLEHCHVRSWVSLGLVHITNAMLAFLCF
LGTFLVQSQPGRYNRARGLTFAMLAYFITWVSFVPLLANVQVAYQPAVQM
GAILVCALGILVTFHLPKCYVLLWLPKLNTQEFFLGRNAKKAADENSGGG
EAAQGHNE

The cDNA of human SAC1 (SEQ ID NO: 4) is:

ATGCTGGGCCCTGCTGTCCTGGGCCTCAGCCTCTGGGCTCTCCTGCACCC
TGGGACGGGGGCCCCATTGTGCCTGTCACAGCAACTTAGGATGAAGGGGG
ACTACGTGCTGGGGGGGCTGTTCCCCCTGGGCGAGGCCGAGGAGGCTGGC
CTCCGCAGCCGGACACGGCCCAGCAGCCCTGTGTGCACCAGGTTCTCCTC
AAACGGCCTGCTCTGGGCACTGGCCATGAAAATGGCCGTGGAGGAGATCA
ACAACAAGTCGGATCTGCTGCCCGGGCTGCGCCTGGGCTACGACCTCTTT
GATACGTGCTCGGAGCCTGTGGTGGCCATGAAGCCCAGCCTCATGTTCCT
GGCCAAGGCAGGCAGCCGCGACATCGCCGCCTACTGCAACTACACGCAGT
ACCAGCCCCGTGTGCTGGCTGTCATCGGGCCCCACTCGTCAGAGCTCGCC
ATGGTCACCGGCAAGTTCTTCAGCTTCTTCCTCATGCCCCAGGTCAGCTA
CGGTGCTAGCATGGAGCTGCTGAGCGCCCGGGAGACCTTCCCCTCCTTCT
TCCGCACCGTGCCCAGCGACCGTGTGCAGCTGACGGCCGCCGCGGAGCTG
CTGCAGGAGTTCGGCTGGAACTGGGTGGCCGCCCTGGGCAGCGACGACGA
GTACGGCCGGCAGGGCCTGAGCATCTTCTCGGCCCTGGCCTCGGCACGCG
GCATCTGCATCGCGCACGAGGGCCTGGTGCCGCTGCCCCGTGCCGATGAC
TCGCGGCTGGGGAAGGTGCAGGACGTCCTGCACCAGGTGAACCAGAGCAG
CGTGCAGGTGGTGCTGCTGTTCGCCTCCGTGCACGCCGCCCACGCCCTCT
TCAACTACAGCATCAGCAGCAGGCTCTCGCCCAAGGTGTGGGTGGCCAGC
GAGGCCTGGCTGACCTCTGACCTGGTCATGGGGCTGCCCGGCATGGCCCA
GATGGGCACGGTGCTTGGCTTCCTCCAGAGGGGTGCCCAGCTGCACGAGT
TCCCCCAGTACGTGAAGACGCACCTGGCCCTGGCCACCGACCCGGCCTTC
TGCTCTGCCCTGGGCGAGAGGGAGCAGGGTCTGGAGGAGGACGTGGTGGG
CCAGCGCTGCCCGCAGTGTGACTGCATCACGCTGCAGAACGTGAGCGCAG
GGCTAAATCACCACCAGACGTTCTCTGTCTACGCAGCTGTGTATAGCGTG
GCCCAGGCCCTGCACAACACTCTTCAGTGCAACGCCTCAGGCTGCCCCGC
GCAGGACCCCGTGAAGCCCTGGCAGCTCCTGGAGAACATGTACAACCTGA
CCTTCCACGTGGGCGGGCTGCCGCTGCGGTTCGACAGCAGCGGAAACGTG
GACATGGAGTACGACCTGAAGCTGTGGGTGTGGCAGGGCTCAGTGCCCAG
GCTCCACGACGTGGGCAGGTTCAACGGCAGCCTCAGGACAGAGCGCCTGA
AGATCCGCTGGCACACGTCTGACAACCAGAAGCCCGTGTCCCGGTGCTCG
CGGCAGTGCCAGGAGGGCCAGGTGCGCCGGGTCAAGGGGTTCCACTCCTG
CTGCTACGACTGTGTGGACTGCGAGGCGGGCAGCTACCGGCAAAACCCAG
ACGACATCGCCTGCACCTTTTGTGGCCAGGATGAGTGGTCCCCGGAGCGA
AGCACACGCTGCTTCCGCCGCAGGTCTCGGTTCCTGGCATGGGGCGAGCC
GGCTGTGCTGCTGCTGCTCCTGCTGCTGAGCCTGGCGCTGGGCCTTGTGC
TGGCTGCTTTGGGGCTGTTCGTTCACCATCGGGACAGCCCACTGGTTCAG
GCCTCGGGGGGGCCCCTGGCCTGCTTTGGCCTGGTGTGCCTGGGCCTGGT
CTGCCTCAGCGTCCTCCTGTTCCCTGGCCAGCCCAGCCCTGCCCGATGCC
TGGCCCAGCAGCCCTTGTCCCACCTCCCGCTCACGGGCTGCCTGAGCACA
CTCTTCCTGCAGGCGGCCGAGATCTTCGTGGAGTCAGAACTGCCTCTGAG
CTGGGCAGACCGGCTGAGTGGCTGCCTGCGGGGGCCCTGGGCCTGGCTGG
TGGTGCTGCTGGCCATGCTGGTGGAGGTCGCACTGTGCACCTGGTACCTG
GTGGCCTTCCCGCCGGAGGTGGTGACGGACTGGCACATGCTGCCCACGGA
GGCGCTGGTGCACTGCCGCACACGCTCCTGGGTCAGCTTCGGCCTAGCGC
ACGCCACCAATGCCACGCTGGCCTTTCTCTGCTTCCTGGGCACTTTCCTG
GTGCGGAGCCAGCCGGGCCGCTACAACCGTGCCCGTGGCCTCACCTTTGC
CATGCTGGCCTACTTCATCACCTGGGTCTCCTTTGTGCCCCTCCTGGCCA
ATGTGCAGGTGGTCCTCAGGCCCGCCGTGCAGATGGGCGCCCTCCTGCTC
TGTGTCCTGGGCATCCTGGCTGCCTTCCACCTGCCCAGGTGTTACCTGCT
CATGCGGCAGCCAGGGCTCAACACCCCCGAGTTCTTCCTGGGAGGGGGCC
CTGGGGATGCCCAAGGCCAGAATGACGGGAACACAGGAAATCAGGGGAAA
CATGAGTGA

The polypeptide sequence of human SAC1 substantially from the translated region of the human cDNA (SEQ ID NO: 5) is:

MLGPAVLGLSLWALLHPGTGAPLCLSQQLRMKGDYVLGGLFPLGEAEEAG
LRSRTRPSSPVCTRFSSNGLLWALAMKMAVEEINNKSDLLPGLRLGYDLF
DTCSEPVVAMKPSLMFLAKAGSRDIAAYCNYTQYQPRVLAVIGPHSSELA
MVTGKFFSFFLMPQVSYGASMELLSARETFPSFFRTVPSDRVQLTAAAEL
LQEFGWNWVAALGSDDEYGRQGLSJFSALASARGICIAHEGLVPLPRADD
SRLGKVQDVLHQVNQSSVQVVLLFASVHAAHALFNYSISSRLSPKVWVAS
EAWLTSDLVMGLPGMAQMGTVLGFLQRGAQLHEFPQYVKTHLALATDPAF
CSALGEREQGLEEDVVGQRCPQCDCITLQNVSAGLNHHQTFSVYAAVYSV
AQALHNTLQCNASGCPAQDPVKPWQLLENMYNLTFHVGGLPLRFDSSGNV
DMEYDLKLWVWQGSVPRLHDVGRFNGSLRTERLKIRWHTSDNQKPVSRCS
RQCQEGQVRRVKGFHSCCYDCVDCEAGSYRQNPDDIACTFCGQDEWSPER
STRCFRRRSRFLAWGEPAVLLLLLLLSLALGLVLAALGLFVHHRDSPLVQ
ASGGPLACFGLVCLGLVCLSVLLFPGQPSPARCLAQQPLSHLPLTGCLST
LFLQAAEIFVESELPLSWADRLSGCLRGPWAWLVVLLAMLVEVALCTWYL
VAFPPEVVTDWIIMLPTEALVHCRTRSWVSFGLAHATNATLAFLCFLGTF
LVRSQPGRYNRARGLTFAMLAYFITWVSFVPLLANVQVVLRPAVQMGALL
LCVLGILAAFHLPRCYLLMRQPGLNTPEFFLGGGPGDAQGQNPGNTGNQG
RHE

### III. SAC1 Is a G-Protein Coupled Receptor

The evidence that SAC is a G-protein coupled receptor (GPCR) comes from its sequence homology to other GPCR and the structure predicted for the amino acid sequence.

GPCRs (also known as 7-transmembrane receptors) bind extracellular ligands and transduce signals into the cell by coupling to intracellular G-proteins. GPCRs can be subdivided into more than 30 families on the basis of their ligands. Sac is most closely allied by sequence homology with the Ca⁺⁺-sensing, metabotropic receptors.

Proteins often contain several modules or domains, each with a distinct evolutionary origin and function. When the Sac cDNA sequence is queried against the Conserved Domain Database at NCBI, the following results are obtained:

| | | Score | E |
|---|---|---|---|
| Sequences producing significant alignments: | | (bits) | Value |
| Gnl\|Pfam\|pfam01094 | ANF_receptor, Receptor family | 145 | 73−36 |
| | ligand binding region | | |
| Gnl\|Pfam\|pfam00003 | 7tm_3, 7-transmembrane | 87.0 | 3e−18 |
| | receptor (metabotropic glutamate | | |
| | family) | | |

Note
the ANF_receptor family contains the metabotropic and calcium-sensing families of GCPs.

The closest sequence homology of the mouse SAC gene is to the Ca⁺⁺ sensing receptors, all of which are GCPRs. An alignment between a calcium sensing GPCR (BAA09453) is shown in FIG. 5.

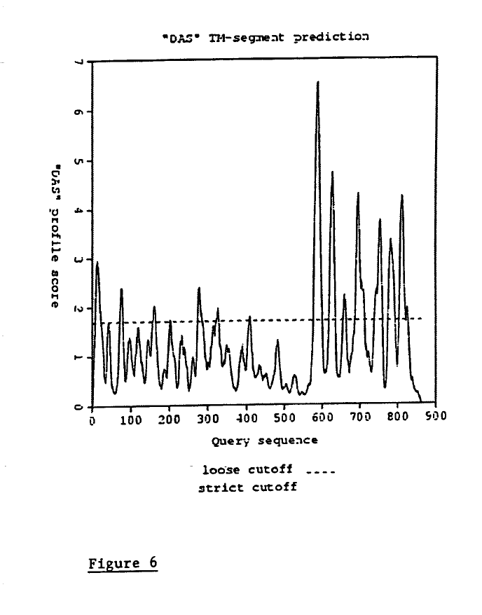

As described above, all GPCRs have a characteristic 7-transmembrane domain. FIG. 6 is a plot of the transmembrane domains of SAC1.

TABLE 1
Genes Predicted From the Sac Nonrecombinant Interval and Expression Data From NCBI

| | | | How | |
|---|---|---|---|---|
| | | | Many | |
| | | | EST | |
| | | Size | From | |
| N | Gene or EST | (aa) | Tongue? | Suggested Protein Function |
| 1 | Cyclin ania 6a | ˜425 | 0/36 | Potentially involved in differentiation and neural plasticity |
| 2 | Slm1 | ˜189 | 0/29 | Slm-1 is a Src substrate during mitosis |
| 3 | AA404005 | 446 | 0/61 | Expressed in kidney |
| 4 | Disheveled | 769 | 0/6 | Segment polarity gene; knockouts have a behavioral phenotype |
| 5 | Sac | 746¹ | 0/0² | Sweet receptor |
| 6 | Mm.25556 | 216 | 0/5 | Weakly similar to Physcomitrella patens glyceraldehyde 3-phosphate dehydrogenase in |
| | | | | C. elegans |
| 7 | Mm.135238 | 524 | 0/5 | Expressed in mammary gland and spleen |
| 8 | AA435261 | 328 | 0/1 | Expressed in mouse two cell |
| 9 | Centaurin | 791 | 0/1 | Regulators of membrane traffic and the actin cytoskeleton |
| | beta 2# | | | |
| 10 | Voltage gated | 170 | 0/0 | Gumarin reduces the perception of sweet, and may work by blocking sodium channels |
| | Na⁺ channel # | | | (Fletcher J. I., Chapman B. E., Mackay J. P., Howden M. E., and King G. F. The structure of |
| | | | | versutoxin (delta-atracotoxin-Hv1) provides insights into the binding of site 3 neurotoxins to |
| | | | | the voltage-gated sodium channel. Structure, 1997; 5: 1525-1535) |
| 11 | Ubc6p | 597 | 0/32 | Essential for the degradation of misfolded and regulated proteins in the endoplasmic reticulum |
| | | | | lumen and membrane |
| 12 | Mm.29140 | 402 | 0/2 | Weakly similar to collagen alpha 1(XVIII) chain |
| The genomic sequence from AF185591 and RPCI-23-118E21, between the markers that flank the Sac nonrecombiant interval, was identified. The repetitive and low complexity sequences were removed, using Repeatmasker (Smit F. and Green P. Repeatmasker). The resulting sequence was analyzed by GENSCAN, which predicted 12 proteins. Of these 12 predicted proteins, one GENSCAN predicted protein was a chimera between two genes (cyclin ania 6a and Slm1). | | | | |
| # (These sequences were separated into their respective sequences.) The predicted proteins were submitted to a TBLASTN search through the nr and the mouse EST database at NCBI. Of the 12 predicted proteins, four were named genes, two genes were similar to other named genes (Centaurin beta 2 and the voltage gated Na⁺ channel) and are denoted with an #. Three of the predicted proteins were represented as ESTs, and had Unigene cluster numbers. The remaining two | | | | |
| # predicted genes were identical to previously isolated mouse ESTs. When each predicted protein was blasted against the mouse EST database, the number of ESTs from tongue were compared with the number from other tissue sources. No ESTs from these genes appeared in the mouse EST database at NCBI. | | | | |

¹Note that the GENSCAN prediction is not accurate; sequencing of the cDNA indicates Sac is 858 aa.
²Note that TR1-like is expressed in tongue as detected by RT-PCR. Previously named genes are in italics, and ESTs or EST clusters in plain text.

### IV. The Sac Locus and the Gpr98 Sweet Taste Receptor Gene

A substantial effort has been devoted to positional cloning of a locus on distal Chr 4 with a major effect on sweetener intake. This locus has been previously described as the Sac (saccharin preference) locus, and it also explains ˜8% of the phenotypic variance in ethanol preferences within the B6×129 F₂ generation.

Details on positional cloning of the Sac locus are found above.

The effects of SAC1 (Gpr98) on ethanol intake Two lines of evidence point to the involvement of Gpr98 in ethanol intake. First, 129.B6-Sac congenic mice homozygous for a 194-kb donor fragment from the B6 strain consumed more 10% ethanol solution than did congenic mice without the donor fragment (1.50±0.15 and 1.19±0.11 mL/day, respectively; p<0.05, one-tailed t-test). Second, ethanol preference was related to sequence variations of Gpr98. Analysis of Gpr98 sequences from genealogically remote or unrelated mouse strains indicated the presence of two haplotypes of single nucleotide polymorphisms within the Gpr98 locus. One, ‘B6-like’ haplotype, was found in mouse strains with elevated sweetener preference and the other, ‘129-like’ haplotype, was found in strains relatively indifferent to sweeteners as described above. Preferences for 10% ethanol for the same mouse strains were studied as described in Abstr. of the 23th RSA Meeting (June 2000, Denver, Colo.). We found that strains with the ‘B6-like’ haplotype had higher preferences for 10% ethanol (20±4%, n=14, strains C57BL/6J, C57L/J, CAST, FVB/NJ, KK/HIJ, NOD/LtJ, NZB/B1NJ, P/J, RBF/DnJ, RF/J, SEA/GnJ, SJL/J, SPRET/Ei and SWR/J) compared with strains having the ‘129-like’ haplotype (12±2%, n=10, p<0.05, one-tailed t-test, strains 129P3/J, AKR/J, BALB/c, BUB/BnJ, C3H/HeJ, CBA/J, DBA/2J, LP/J, PL/J and RIIIS/J).

### V. Preparation of Recombinant or Chemically Synthesized Nucleic Acids, Vectors, Transformation, Host-Cells

Large amounts of the polynucleotides of the present invention may be produced by replication in a suitable host cell. Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines. The purification of nucleic acids produced by the methods of the present invention is described, e.g., in Ausubel et al., Current Protocols in Molecular Biology, Vol. 1-2, John Wiley & Sons, 1992 and Sambrook et al., Molecular Cloning A Laboratory Manual, 2nd Ed., Vols. 1-3, Cold Springs Harbor Press, 1989.

The polynucleotides of the present invention may also be produced by chemical synthesis, e.g., by the phosphoramidite method or the triester method, and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native SAC1 protein or from other receptors or from secreted polypeptides of the same or related species, which allow the protein to cross and/or lodge in cell membranes, and thus attain its functional topology, or be secreted from the cell. Such vectors may be prepared by means of standard recombinant techniques well-known in the art and discussed, for example, in Sambrook et al., 1989 or Ausubel et al., 1992.

An appropriate promoter and other necessary vector sequences will be selected so as to be functional in the host, and may include, when appropriate, those naturally associated with SAC1 genes. Examples of workable combinations of cell lines and expression vectors are described in Sambrook et al., 1989 or Ausubel et al., 1992. Many useful vectors are known in the art and may be obtained from commercial vendors. Promoters such as the trp, lac and phage promoters, TRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. In addition, the construct may be joined to an amplifiable gene so that multiple copies of the gene may be made. For appropriate enhancer and other expression control sequences, see also Enhancers and Eukaryotic Gene Expression, New York: Cold Spring Harbor Press, 1983. See also, e.g., U.S. Pat. Nos. 5,691,198; 5,735,500; 5,747,469 and 5,436,146.

Expression and cloning vectors will likely contain a selectable marker, a gene encoding a protein necessary for survival or growth of a host cell transformed with the vector. The presence of this gene ensures growth of only those host cells which express the inserts. Typical selection genes encode proteins that (a) confer resistance to antibiotics or other toxic substances, e.g., ampicillin, neomycin, methotrcxate, etc.; (b) complement auxotrophic deficiencies; or (c) supply critical nutrients not available from complex media, e.g., the gene encoding D-alanine racemase for Bacilli. The choice of the proper selectable marker will depend on the host cell, and appropriate markers for different hosts are well-known in the art.

The vectors containing the nucleic acids of interest can be transcribed in vitro, and the resulting RNA introduced into the host cell by well-known methods, e.g., by injection, or the vectors can be introduced directly into host cells by methods well-known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described above are meant to also include the progeny of such cells.

Large quantities of the nucleic acids and polypeptides of the present invention may be prepared by expressing the SAC1 nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of the proteins of the present invention. Propagation of mammalian cells in culture is per se well-known. Examples of commonly used mammalian host cell lines are VERO and HeLa cells, Chinese hamster ovary (CHO) cells, and W138, BHK, and COS cell lines. An example of a commonly used insect cell line is SF9. However, it will be appreciated by the skilled practitioner that other cell lines may be appropriate, e.g., to provide higher expression, desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

### VI. Diagnosis or Screening

Genetic analysis of obesity and diabetes and alcoholism or alcohol consumption is often complicated by the lack of a simple diagnostic mark. For example, currently there is no single diagnostic marker for the diagnosis of obesity. Sequence variation of the SAC1 locus may indicate a predisposition to diabetes, obesity, and alcoholism and may provide a diagnostic mark.

In order to detect the presence of a SAC1 allele predisposing an individual to obesity, diabetes, or alcoholism, a biological sample may be prepared and analyzed for the presence or absence of susceptibility alleles of SAC1. Results of these tests and interpretive information may be returned to the health care professionals for communication to the tested individual. Such diagnoses may be performed by diagnostic laboratories. In addition, diagnostic kits may be manufactured and available to health care providers or to private individuals for self-diagnosis.

A basic format for sequence or expression analysis is finding sequences in DNA or RNA extracted from affected family members which create abnormal SAC1 gene products or abnormal levels of SAC1 gene product. The diagnostic or screening method may involve amplification or molecular cloning of the relevant SAC1 sequences. For example, PCR based amplification may be used. Once amplified, the resulting nucleic acid can be sequenced or used as a substrate for DNA probes. Primers and probes specific for the SAC1 gene sequences may be used to identify SAC1 alleles.

The pairs of single-stranded DNA primers can be annealed to sequences within or surrounding the SAC1 gene in order to prime amplifying DNA synthesis of the SAC1 gene itself. The set of primers may allow synthesis of both intron and exon sequences. Allele-specific primers can also be used. Such primers anneal only to particular SAC1 mutant alleles, and thus will only amplify a product in the presence of the mutant allele as a template.

In order to facilitate subsequent cloning of amplified sequences, primers may have restriction enzyme site sequences appended to their 5′ ends. Thus, all nucleotides of the primers are derived from SAC1 sequences or sequences adjacent to SAC1, except for the few nucleotides necessary to form a restriction enzyme site. Such enzymes and sites are well-known in the art. The primers themselves can be synthesized using techniques which are well-known in the art. Generally, the primers can be made using oligonucleotide synthesizers which are commercially available.

The biological sample to be analyzed, such as blood, may be treated, if desired, to extract the nucleic acids. The sample nucleic acid may be prepared in various ways to facilitate detection of the target sequence; e.g., denaturation, restriction digestion, electrophoresis or dot blotting. The region of interest of the target nucleic acid is usually at least partially single-stranded to form hybrids with the probe. If the sequence is double-stranded, the sequence will probably need to be denatured. The target nucleic acid may be also be fragmented to reduce or eliminate the formation of secondary structures. The fragmentation may be performed using a number of methods, including enzymatic, chemical, thermal cleavage or degradation. For example, fragmentation may be accomplished by heat/Mg²⁺ treatment, endonuclease (e.g., DNAase 1) treatment, restriction enzyme digestion, shearing (e.g., by ultrasound) or NaOH treatment.

Many genotyping and expression monitoring methods have been described previously. In general, target nucleic acid and probe are incubated under conditions which forms hybridization complex between the probe and the target sequence. The region of the probes which is used to bind to the target sequence can be made completely complementary to the targeted region of the SAC1 locus. Therefore, high stringency conditions may be desirable in order to prevent false positives. However, conditions of high stringency are typically used if the probes are complementary to regions of the chromosome which are unique in the genome. The stringency of hybridization is determined by a number of factors during hybridization and during the washing procedure, including temperature, ionic strength, base composition, probe length, and concentration of formamide. Under certain circumstances, the formation of higher order hybrids, such as triplexes, quadraplexes, etc. may be desired to provide the means of detecting target sequences.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinase reaction), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labeled moiety.

Two-step label amplification methodologies are known in the art. These assays work on the principle that a small ligand (such as digoxigenin, biotin, or the like) is attached to a nucleic acid probe capable of specifically binding SAC1.

In one example, the small ligand attached to the nucleic acid probe is specifically recognized by an antibody-enzyme conjugate. In one embodiment of this example, digoxigenin is attached to the nucleic acid probe. Hybridization is detected by an antibody-alkaline phosphatase conjugate which turns over a chemiluminescent substrate. In a second example, the small ligand is recognized by a second ligand-enzyme conjugate that is capable of specifically complexing to the first ligand. A well-known embodiment of this example is the biotin-avidin type of interactions.

It is also contemplated within the scope of this invention that the nucleic acid probe assays of this invention will employ a cocktail of nucleic acid probes capable of detecting SAC1. Thus, in one example to detect the presence of SAC1 in a biological sample, more than one probe complementary to SAC1 is employed.

Predisposition to diabetes, obesity, or alcoholism can be ascertained by testing any fluid or tissue of a human for sequence variations of the SAC1 gene. For example, a person who has inherited a germline SAC1 mutation would be prone to develop obesity, diabetes, or alcoholism. This can be determined by testing DNA from any tissue of the person's body. Most simply, blood can be drawn and DNA extracted from the cells of the blood. In addition, prenatal diagnosis can be accomplished by testing fetal cells, placental cells or amniotic cells for mutations of the SAC1 gene.

The most definitive test for mutations in a candidate locus is to directly compare genomic SAC1 sequences from obese, diabetic, or alcoholic patients, with those from a control population. Alternatively, one could sequence messenger RNA after amplification, e.g., by PCR, thereby eliminating the necessity of determining the exon structure of the candidate gene.

Sequence variations from diabetic, obese, or alcoholic patients falling outside the coding region of SAC1 can be detected by examining the non-coding regions, such as introns and regulatory sequences near or within the SAC1 gene. An early indication that mutations in noncoding regions are important may come from Northern blot experiments that reveal messenger RNA molecules of abnormal size or abundance in obese or diabetic patients as compared to control individuals.

Alteration of SAC1 mRNA expression can be detected by any techniques known in the art (see above). These include Northern blot analysis, PCR amplification, RNase protection, and gene chip analysis. Diminished mRNA expression indicates an alteration of the wild-type SAC1 gene.

The diabetic, obese, or alcoholic condition can also be detected on the basis of the alteration of wild-type SAC1 polypeptide. For example, the presence of a SAC1 gene variant, which produces a protein having a loss of function, or altered function, may directly correlate to an increased risk of obesity or diabetes. Such variation can be determined by sequence analysis in accordance with conventional techniques. For example, antibodies (polyclonal or monoclonal) may be used to detect differences in, or the absence of, SAC1 polypeptides. Antibodies may immunoprecipitate SAC1 proteins from solution as well as react with SAC1 protein on Western or immunoblots of polyacrylamide gels. Antibodies may also detect SAC1 proteins in paraffin or frozen tissue sections, using immunocytochemical techniques. Immunoassay include, for example, enzyme linked immunosorbent assays (ELISA), radioimmunoassays (RIA), immunoradiometric assays (IRMA) and immunoenzymatic assays (IEMA), sandwich assays, etc.

Functional assays, such as protein binding determinations, can be used. Finding a mutant SAC1 gene product indicates alteration of a wild-type SAC1 gene.

### VII. Drug, Sweetener, and Alcohol Preference Screening

This invention is also useful for screening compounds by using the SAC1 polypeptide or binding fragment thereof in any of a variety of drug, sweetener, and alcohol screening techniques.

The SAC1 polypeptide or fragment employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, for the formation of complexes between a SAC1 polypeptide or fragment and the agent being tested, or examine the degree to which the formation of a complex between a SAC1 polypeptide or fragment and a known ligand is interfered with by the agent being tested.

Thus, the present invention provides methods of screening for drugs and sweeteners comprising contacting such an agent with a SAC1 polypeptide or fragment thereof and assaying (i) for the presence of a complex between the agent and the SAC1 polypeptide or fragment, or (ii) for the presence of a complex between the SAC1 polypeptide or fragment and a ligand, by methods well-known in the art. In such competitive binding assays the SAC1 polypeptide or fragment is typically labeled. Free SAC1 polypeptide or fragment is separated from that present in a protein:protein complex, and the amount of free (i.e., uncomplexed) label is a measure of the binding of the agent being tested to SAC1 or its interference with SAC1:ligand binding, respectively.

Other suitable techniques for drug, sweetener, and alcohol screening may provide high throughput screening for compounds having suitable binding affinity to the SAC1 polypeptides. For example, large numbers of different small peptide test compounds are synthesized on a solid substrate, such as plastic pins or some other surface. The peptide test compounds are reacted with SAC1 polypeptide and washed. Bound SAC1 polypeptide is then detected by methods well-known in the art.

Purified SAC1 can be coated directly onto plates for use in the aforementioned drug screening techniques. However, non-neutralizing antibodies to the polypeptide can be used to capture antibodies to immobilize the SAC1 polypeptide on the solid phase.

This invention also contemplates the use of competitive drug, sweetener, and alcohol screening assays in which neutralizing antibodies capable of specifically binding the SAC1 polypeptide compete with a test compound for binding to the SAC1 polypeptide or fragments thereof. In this manner, the antibodies can be used to detect the presence of any peptide which shares one or more antigenic determinants of the SAC1 polypeptide.

A further technique for drug, sweetener, and alcohol screening involves the use of host eukaryotic cell lines or cells which have a nonfunctional SAC1 gene. These host cell lines or cells are defective at the SAC1 polypeptide level. The host cell lines or cells are grown in the presence of the drug, sweetener, or alcohol compound. The rate of growth of the host cells is measured to determine if the compound is capable of regulating the growth of SAC1 defective cells.

Briefly, a method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Prior to or as well as being screened for modulation of activity, test substances may be screened for ability to interact with the polypeptide, e.g., in a yeast two-hybrid system. This system may be used as a coarse screen prior to testing a substance for actual ability to modulate activity of the polypeptide. Alternatively, the screen could be used to screen test substances for binding to a SAC1 specific binding partner, or to find mimetics of a SAC1 polypeptide.

### VIII. Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. In one approach, one first determines the three-dimensional structure of a protein of interest (e.g., SAC1 polypeptide) or, for example, of the SAC1-receptor or ligand complex, by x-ray crystallography, by computer modeling or most typically, by a combination of approaches. Less often, useful information regarding the structure of a polypeptide may be gained by modeling based on the structure of homologous proteins. An example of rational drug design is the development of HIV protease inhibitors. In addition, peptides (e.g., SAC1 polypeptide) are analyzed by an alanine scan. In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Thus, one may design drugs which have, e.g., improved SAC1 polypeptide activity or stability or which act as inhibitors, agonists, antagonists, etc. of SAC1 polypeptide activity. By virtue of the availability of cloned SAC1 sequences, sufficient amounts of the SAC1 polypeptide may be made available to perform such analytical studies as x-ray crystallography. In addition, the knowledge of the SAC1 protein sequence provided herein will guide those employing computer modeling techniques in place of, or in addition to x-ray crystallography.

Following identification of a substance which modulates or affects polypeptide activity, the substance may be investigated further. Furthermore, it may be manufactured and/or used in preparation, i.e., manufacture or formulation, or a composition such as a medicament, pharmaceutical composition or drug. These may be administered to individuals.

Thus, the present invention extends in various aspects not only to a substance identified using a nucleic acid molecule as a modulator of polypeptide activity, in accordance with what is disclosed herein, but also a pharmaceutical composition, medicament, drug or other composition comprising such a substance, a method comprising administration of such a composition comprising such a substance, a method comprising administration of such a composition to a patient, e.g., for treatment of diabetes, obesity or alcohol consumption, use of such a substance in the manufacture of a composition for administration, e.g., for treatment of diabetes or alcohol consumption, and a method of making a pharmaceutical composition comprising admixing such a substance with a pharmaceutically acceptable excipient, vehicle or carrier, and optionally other ingredients.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g., by substituting each residue in turn. Alanine scans of peptide are commonly used to refine such peptide motifs. These parts or residues constituting the active region of the compound are known as its pharmacophore.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially used where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic(s) found by this approach can then be screened to see whether they have the target property, or to what extent they exhibit it. Further optimization or modification can then be carried out to arrive at one or more final mimetics for in vivo or clinical testing.

### IX. Gene Therapy

According to the present invention, a method is also provided of supplying wild-type SAC1 function to a cell which carries mutant SAC1 alleles. The wild-type SAC1 gene or a part of the gene may be introduced into the cell in a vector such that the gene remains extrachromosomal. In such a situation, the gene will be expressed by the cell from the extra chromosomal location. More preferred is the situation where the wild-type SAC1 gene or a part thereof is introduced into the mutant cell in such a way that it recombines with the endogenous mutant SAC1 gene present in the cell. Such recombination requires a double recombination event which results in the correction of the SAC1 gene mutation. Vectors for introduction of genes both for recombination and for extrachromosomal maintenance are known in the art, and any suitable vector may be used. Methods for introducing DNA into cells such as electroporation, calcium phosphate coprecipitation and viral transduction are known in the art, and the choice of method is within the competence of skilled practitioners.

As generally discussed above, the SAC1 gene or fragment, where applicable, may be employed in gene therapy methods in order to increase the amount of the expression products of such genes in diabetic or obese cells. Such gene therapy is particularly appropriate, in which the level of SAC1 polypeptide is absent or compared to normal cells. It may also be useful to increase the level of expression of a given SAC1 gene even in those situations in which the mutant gene is expressed at a “normal” level, but the gene product is not fully functional.

Gene therapy would be carried out according to generally accepted methods, for example, as described by Therapy for Genetic Diseases, T. Friedman, ed. Oxford University Press, 1991. Cells from a patient would be first analyzed by the diagnostic methods described above, to ascertain the production of SAC1 polypeptide in these cells. A virus or plasmid vector, containing a copy of the SAC1 gene linked to expression control elements and capable of replicating inside the sample cells, is prepared. Suitable vectors are known, such as disclosed in PCT publications WO 93/07282 and U.S. Pat. Nos. 5,252,479, 5,691,198, 5,747,469, 5,436,146 and 5,753,500. The vector is then injected into the patient.

Gene transfer systems known in the art may be useful in the practice of the gene therapy methods of the present invention. These include viral and nonviral transfer methods. A number of viruses have been used as gene transfer vectors, including papovaviruses, e.g., SV40, adenovirus, vaccinia virus, adeno-associated virus, herpes viruses including HSV and EBV; lentiviruses, Sindbis and Semliki Forest virus, and retroviruses of avian, murine, and human origin. Most human gene therapy protocols have been based on disabled murine retroviruses.

Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer. Viral-mediated gene transfer can be combined with direct in vivo gene transfer using liposome delivery, allowing one to direct the viral vectors to the affected cells and not into the surrounding nondividing cells. Alternatively, the retroviral vector producer cell line can be injected into affected cells. Injection of producer cells would then provide a continuous source of vector particles.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged. For other techniques for the delivery of adenovirus based vectors see U.S. Pat. Nos. 5,691,198; 5,747,469; 5,436,146 and 5,753,500.

Liposome/DNA complexes have been shown to be capable of mediating direct in vivo gene transfer. While in standard liposome preparations the gene transfer process is nonspecific, localized in vivo uptake and expression may be accomplished following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes SAC1, expression will produce SAC1. If the polynucleotide encodes an antisense polynucleotide or a ribozyme, expression will produce the antisense polynucleotide or ribozyme. Thus in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Receptor-mediated gene transfer, for example, may be accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. One appropriate receptor/ligand pair may include the estrogen receptor and its ligand, estrogen (and estrogen analogues). These ligand-DNA conjugates can be injected directly into the blood if desired and are directed to the target tissue where receptor binding and internalization of the DNA-protein complex occurs. To overcome the problem of intracellular destruction of DNA, coinfection with adenovirus can be included to disrupt endosome function.

### X. Peptide Therapy

Peptides which have SAC1 activity can be supplied to cells which carry mutant or missing SAC1 alleles. Protein can be produced by expression of the cDNA sequence in bacteria, for example, using known expression vectors. Alternatively, SAC1 polypeptide can be extracted from SAC1-producing mammalian cells. In addition, the techniques of synthetic chemistry can be employed to synthesize SAC1 protein. Any of such techniques can provide the preparation of the present invention which comprises the SAC1 protein. Preparation is substantially free of other human proteins. This is most readily accomplished by synthesis in a microorganism or in vitro.

Active SAC1 molecules can be introduced into cells by microinjection or by use of liposomes, for example. Alternatively, some active molecules may be taken up by cells, actively or by diffusion. Extra-cellular application of the SAC1 gene product may be sufficient. Molecules with SAC1 activity (for example, peptides, drugs or organic compounds) may also be used to effect such a reversal. Modified polypeptides having substantially similar function are also used for peptide therapy.

### XI. Transformed Hosts

Similarly, cells and animals which carry a mutant SAC1 allele can be used as model systems to study and test for substances which have potential as therapeutic agents. These may be isolated from individuals with SAC1 mutations, either somatic or germline. Alternatively, the cell line can be engineered to carry the mutation in the SAC1 allele.

Animals for testing therapeutic agents can be selected after mutagenesis of whole animals or after treatment of germline cells or zygotes. Such treatments include insertion of mutant SAC1 alleles, usually from a second animal species, as well as insertion of disrupted homologous genes. Alternatively, the endogenous SAC1 gene of the animals may be disrupted by insertion or deletion mutation or other genetic alterations using conventional techniques to produce knockout or transplacement animals. A transplacement is similar to a knockout because the endogenous gene is replaced, but in the case of a transplacement the replacement is by another version of the same gene. After test substances have been administered to the animals, the phenotype must be assessed. If the test substance prevents or suppresses the disease, then the test substance is a candidate therapeutic agent for the treatment of disease. These animal models provide an extremely important testing vehicle for potential therapeutic products.

In one embodiment of the invention, transgenic animals are produced which contain a functional transgene encoding a functional SAC1 polypeptide or variants thereof. Transgenic animals expressing SAC1 transgenes, recombinant cell lines derived from such animals and transgenic embryos may be useful in methods for screening for and identifying agents that induce or repress function of SAC1. Transgenic animals of the present invention also can be used as models for studying indications such as diabetes.

In one embodiment of the invention, a SAC1 transgene is introduced into a non-human host to produce a transgenic animal expressing a human or murine SAC1 gene. The transgenic animal is produced by the integration of the transgene into the genome in a manner that permits the expression of the transgene. Methods for producing transgenic animals are generally described in U.S. Pat. No. 4,873,191 and in Manipulating the Mouse Embryo; A Laboratory Manual, 2nd edition (eds., Hogan, Beddington, Costantimi and Long, New York: Cold Spring Harbor Laboratory Press, 1994).

It may be desirable to replace the endogenous SAC1 by homologous recombination between the transgene and the endogenous gene; or the endogenous gene may be eliminated by deletion as in the preparation of “knock-out” animals. Typically, a SAC1 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a mutant form of the polypeptide.

As noted above, transgenic animals and cell lines derived from such animals may find use in certain testing experiments. In this regard, transgenic animals and cell lines capable of expressing wild-type or mutant SAC1 may be exposed to test substances. These test substances can be screened for the ability to reduce overexpression of wild-type SAC1 or impair the expression or function of mutant SAC1.

### XII. Pharmaceutical Compositions and Routes of Administration

The SAC1 polypeptides, antibodies, peptides and nucleic acids of the present invention can be formulated in pharmaceutical compositions, which are prepared according to conventional pharmaceutical compounding techniques. See, for example, Remington's Pharmaceutic. Sciences, 18th Ed. (Easton, Pa.: Mack Publishing Co., 1990). The composition may contain the active agent or pharmaceutically acceptable salts of the active agent. These compositions may comprise, in addition to one of the active substances, a pharmaceutically acceptable excipient, carrier, buffer, stabilizer or other materials well-known in the art. Such materials should be nontoxic and should not interfere with the efficacy of the active ingredient. The carrier may take a wide variety of forms depending on the form of preparation desired for administration, e.g., intravenous, oral, intrathecal, epineural or parenteral.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g., decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used to deliver the active agent more specifically to certain types of cell, by the use of targeting systems such as antibodies or cell specific ligands. Targeting may be desirable for a variety of reasons, e.g., if the agent is unacceptably toxic, or if it would otherwise require too high a dosage, or if it would not otherwise be able to enter the target cells.

Instead of administering these agents directly, they could be produced in the target cell, e.g., in a viral vector such as described above or in a cell based delivery system such as described in U.S. Pat. No. 5,550,050 and PCT publications WO 92/19195, WO 94/25503, WO 95/01203, WO 95/05452, WO 96/02286, WO 96/02646, WO 96/40871, WO 96/40959 and WO 97/12635, designed for implantation in a patient. The vector could be targeted to the specific cells to be treated, or it could contain regulatory elements which are more tissue specific to the target cells. The cell based delivery system is designed to be implanted in a patient's body at the desired target site and contains a coding sequence for the active agent. Alternatively, the agent could be administered in a precursor form for conversion to the active form by an activating agent produced in, or targeted to, the cells to be treated. See for example, EP 425,731A and WO 90/07936.

### EXAMPLES

The following examples further illustrate the present invention. These examples are intended merely to be illustrative of the present invention and are not to be construed as being limiting.

### Example 1

Animal care and maintenance. All animal protocols used in these studies were approved by the Monell Institutional Animal Care and Use Committee. Mice were housed in individual cages in a temperature-controlled room at 23° C. on a 12-hour light:12-hour dark cycle. The animals had free access to deionized water and Teklad Rodent Diet 8604 (Harlan Teklad, Madison, Wis.).

### Example 2

Breeding of F2 and partially congenic mice. C57BL6/ByJ (B6) and 129P3/J (formerly named 129/J; abbreviated here as 129) mice were purchased from The Jackson Laboratory. The B6 and 129 mice were outcrossed to produce the first filial generation of hybrids (F₁), and these were intercrossed to produce the second hybrid generation (F₂, n=629).

To create the partially congenic lines, the F₂ mice were genotyped with several markers on the distal part of chromosome 4, and a few F₂ mice with recombinations in this region were used as founders of strains partially congenic with the 129 strain. These F₂ founders were backcrossed to the 129 strain to produce the N₂ generation. Mice from this and subsequent backcross generations were phenotyped using 96-hour two-bottle tests with saccharin solutions, and genotyped using markers on distal chromosome 4 and on other autosomes. Mice with high saccharin intake (with a fragment of distal chromosome 4 from the B6 strain and homozygous for 129 alleles of markers on other chromosomes) were selected for subsequent backcrossing. This marker-assisted selection resulted in a segregating 129.B6-Sac partially congenic strain. Three strains were created, with different overlapping fragments containing the SAC1 gene.

### Example 3

Testing of sweet preference in the F2 and partially congenic mice. Consumption of 120 mM sucrose and 17 mM saccharin (Sigma Chemical Company, St. Louis, Mo.) was measured in individually caged mice using 96-hour two-bottle tests, with water as the second choice. The positions of the tubes were switched every 24 hours. Fluid intakes were expressed per 30 g of body weight (the approximate weight of an adult mouse) per day, or as a preference score (ratio of average daily solution intake to total fluid intake, in percent).

### Example 4

Genotyping of F2 mice and linkage analysis. Genomic DNA was purified from mouse tails by NaOH/Tris (Beier, personal communication; Truett G. E. et al., Preparation of PCR-quality mouse genomic DNA with hot sodium hydroxide and tris (HotSHOT) [In Process Citation]. Biotechniques, 2000;29:52, 54), or the phenol/chloroform method. All F2 mice were genotyped with all available polymorphic microsatellite markers (Research Genetics, Huntsville, Ala.) known to map near the SAC1 region with a protocol modified slightly from that described by Dietrich W. et al., A genetic map of the mouse suitable for typing intraspecific crosses. Genetics, 1991; 131;423-447. The markers tested are as follows: D4Mit190, D4Mit42, D4Mit254, and D4Mit256. Analysis of this framework map using MAPMAKER/QTL 1.1 (Lander E. et al. MAPMAKER: An interactive complex package for constructing primary linkage maps of experimental and natural populations. Genomics, 1987;1:174-181), indicated that Sac mapped distal to D4Mit256, and therefore all available STS and EST were tested by SSCP (Orita M., Iwahana H., Kanazawa H., Hayashi K., and Sekiya T. Detection of polymorphisms of human DNA by gel electrophoresis as single-strand conformation polymorphins. Proceedings of the National Academy of Sciences of the USA, 1989:86) or direct sequencing, for polymorphisms between the B6 and 129 strains. Polymorphisms between strains were found for the following markers: D18346, AA410003 (K00231), V2r2, and D4Erdt296E, and the linkage analysis conducted again using these polymorphic makers.

### Example 5

Genotyping of partially congenic mice. Three partially congenic strains of mice were genotyped with all available markers, and those markers with two 129 alleles were excluded from the SAC1 nonrecombinant interval.

### Example 6

Radiation hybrid mapping. To generate additional markers to narrow the Sac nonrecombinant interval, several markers were tested using the T31 RH genome map. Primers from several sequences suggested through survey of the public databases were constructed and DNA from the T31 panel. Results were scored using software at the Jackson Laboratory.

### Example 7

Construction of BAC contig and marker development. To construct a physical map of the SAC1 region, the RPCI-23 BAC library was screened with markers within and near the SAC1 nonrecombinant interval: each marker was tested by whole cell PCR to confirm its presence. Only those markers positive by both hybridization and PCR are shown. Primers for the BAC ends were constructed from sequence obtained through TIGR (www.tigr.org) or by direct sequencing, when necessary. Each positive clone was tested for the presence of each BAC end (if the BAC end contained unique sequence), and the contig oriented using SEGMAP, Version 3.48 (Green E. D. and Green P. Sequence-tagged site (STS) content mapping of human chromosomes: theoretical considerations and early experiences. PCR Methods Appl., 1991;1:77-90). BAC end sequences was amplified in B6 and 129 strains, and analyzed by SSCP or direct sequencing. Those BAC ends polymorphic between 129 and B6 were tested in the recombinant F2 and partially congenic mice, to further narrow the SAC1 nonrecombinant interval.

### Example 8

Amplification of SAC1 and polymorphism detection. After the SAC1 nonrecombinant interval was narrowed to less than 350 kb, a 246 kb BAC was chosen for sequencing which spanned most of the region (RPCI-23-118E21). Within this BAC, there was a gene with homology to other taste receptors. Using 11.8 kb of sequence and the program GENSCAN (Burge C. B. and Karlin S. Finding the genes in genomic DNA. Current Opinion Structural Biology, 1998;8:346-354), a 858 amino acid protein, with 6 exons, was identified. Primers were constructed that amplified this gene, and an additional 2600 nt upstream and 5200 nt downstream were also amplified (primer sequence available upon request). These PCR products were sequenced using genomic DNA from B6 and 129 mouse strains, as well as other strains with either higher (SWR/J, C57L/J, IS, ST/bJ, SEA/GnJ) or lower (DBA/2J, AKR/J, BALB/cByJ) saccharin preference (Lush I. E., The genetics of tasting in mice. VI. Saccharin, acesulfame, dulcin and sucrose. Genet Res., 1989;53:95-99; Lush I. The genetics of bitterness, sweetness, and saltiness in strains of mice. in Genetics of perception and communication, Vol. 3, eds. Wysocki C. and Kare M., New York: Marcel Dekker, 1991:227-235; Lush I. E. and Holland G. The genetics of tasting in mice. V. Glycine and cycloheximide. Genet Res., 1988;52:207-12). Sequences were aligned with Sequencer (Gene Codes, Ann Arbor, Mich.) and the single nucleotide polymorphisms, insertions and deletions identified.

### Example 9

Preparation of tongue cDNA and expression studies. Total RNA was extracted from anterior mouse tongue from the 129 and B6 strains (TRIZOL Reagent; GIBCOBRL). Total RNA (200 ng) was reverse transcribed using the Life Technologies SuperScript Kit. Following the reverse transcription, the samples were amplified using Advantage cDNA PCR Kit (Clontech, Palo Alto, Calif.). Primers were constructed to span exon 2 and 3, so that the genomic and cDNA product size would differ (Primer set 3A; Left-5′TGCATTGGCCAGACTAGAAA3′; Right-5CGGCTGGGCTATGACCTAT′). The expected product size for primer 3A is 418 bp for cDNA and 497 bp for genomic DNA. Single bands of these sizes were excised from the gel, purified and sequenced, confirming the intron-exon boundary and expression of mRNA of this gene in mouse tongue. Primers were then designed to cover the whole cDNA, and, the sequences obtained and aligned, to confirm intron/exon boundaries.

### Example 10

Human gene expression. The human ortholog of the SAC1 gene was examined for mRNA expression in human tongue. Total RNA from human taste papillae was obtained through biopsy, a procedure approved by the Committee on Studies Involving Human Beings at the University of Pennsylvania. The RNA was extracted as described above, reverse transcribed, and amplified, with human specific primers. Two bands were obtained of the expected size for genomic and cDNA. Sequencing of these bands confirmed the SAC1 gene is expressed in human taste papillae.

### Example 11

Tissue Expression of SAC1. Oligonucleotide primers specific for different parts of the SAC1 gene were used to assay different tissues for SAC1 expression as shown in Table 2. Tissue specific cDNA pools were purchased from OriGene Technologies Ltd. Primer pair 3A, amplifies parts of exons 2 and 3, with a small intron to differentiate between PCR product representing genomic DNA versus cDNA. Primer pair 6A amplifies parts of exons 4 and 5. This part of the protein encodes the 7TM domain, and may cross react with other GPCRs expressed in different tissues.

TABLE 2
Expression pattern of SAC1

| Tissue | 3A | 6A |
|---|---|---|
| Brain | − | − |
| Heart | − | − |
| Kidney | + | + |
| Spleen | + | + |
| Thymus | + | + |
| Liver | − | + |
| Stomach | − | + |
| Sm Intestine | − | + |
| Muscle | − | + |
| Lung | − | + |
| Testis | + | + |
| Skin | − | − |
| Adrenal | + | − |
| Pancreas | + | + |
| Uterus | − | − |
| Prostrate | + | + |
| Embryo-8.5 | − | − |
| 9.5 | − | − |
| 12.5 | − | − |
| 19 | + | −/+ |
| Breast-virgin | − | + |
| Pregnant | − | + |
| Lactating | + | + |
| Involuting | − | − |

### Example 12

Primers for the SAC1 Locus (Seq. ID Nos.: 6-651) are:

| Marker | Forward | Reverse | Size, bp | SEQ. ID NO. |
|---|---|---|---|---|
| 28.MMHAP7B4.seq | CACTAGAGCTGCC | CCCTCAGCACCA | 162 | 6-7 |
| | ACCTTCC | CTTTTTGT | | |
| 28.MMHAP7B4.seq | ACAAAAAGTGGTG | CAGGAGACCCA | 163 | 8-9 |
| | CTGAGGG | AAGGATCAA | | |
| AA408705 | GCTTCAGAAAATC | GCATGGGCTATG | 232 | 10-11 |
| | GAGGCAC | ATAGGTGG | | |
| AA408705 | TGTTGATCCCACA | CAGGAAATGTCC | | 12-13 |
| | GCG | ACTTCTGC | | |
| AA409223 | TCTATCTTGCATC | GTGCTGTGACTG | | 14-15 |
| | CAGCC | TGCG | | |
| AA589460 | CGCAGCATTTATT | CCGACCCTTTAG | | 16-17 |
| | TGGAG | GAGACAC | | |
| Agrin4 | TGTGACTTCCTCTT | TGAGCCACTCCA | 156 | 18-19 |
| | CCCCAC | GATGTCAG | | |
| Agrin4 | GTGTGTCAGCATC | CCAACGTGCAGT | 290 | 20-21 |
| | ACTGCCT | CAAGAAAA | | |
| Agrin4 | CGAGAGACAAAG | TTATGAAGGCCC | 263 | 22-23 |
| | TGGTGCTG | TCACCAAC | | |
| Agrin4 | CCAGCTCCTAGAA | GCAGTCTCCCGA | 298 | 24-25 |
| | TTGCCTG | AACAAGTC | | |
| Agrin4 | ATAGAGGAATGG | TACCAGGAGGG | 299 | 26-27 |
| | GTGCGATG | GTCAGTCAG | | |
| Agrin4 | TACAAGCGAGCTG | CCAATCAGCTCG | 271 | 28-29 |
| | ACCAATG | AGTTAGCC | | |
| AgrinA | TGCCATTGTGGAT | GAGTCCGAGGTC | 575 | 30-31 |
| | GTTCACT | GGTCAATA | | |
| AgrinB | GCTGGCTTCTGTA | TATGAGGGTCAA | 577 | 32-33 |
| | GGTCAGG | GGGTCAGG | | |
| AgrinC | CGCTTTGGTGAGA | CATGTGGAGTTG | 573 | 34-35 |
| | ACTAGCC | TGGGAGTG | | |
| AgrinD | AATGGGCAGAAG | TATCAGGGTCTG | 507 | 36-37 |
| | ACAGATGG | TGAAGCCC | | |
| AgrinE | ATACAGGACCCTT | CAGTGTTTCTAG | 587 | 38-39 |
| | TACCCCG | GTCCCCCA | | |
| Agrin | GCCTCTGTCTGCC | ATAATGTTACCT | 594 | 40-41 |
| | ATCTCTC | GCAGGCGG | | |
| AI115523 | CTGGAAACACCCA | CGGGCACATGG | 200 | 42-43 |
| | TGTCCTC | ACACTTTTA | | |
| AI225779 | GAGCATGAAGTGC | CGTAGGTGGCAC | 266 | 44-45 |
| | AAGGTGA | AGTTGAGA | | |
| AI225779 | GCTGTTAGTGAGG | CGTAGGTGGCAC | 104 | 46-47 |
| | TCAGGGC | AGTTGAGA | | |
| AI225779 | GAGCATGAAGTGC | TCATTTTCCTAG | 126 | 48-49 |
| | AAGGTGA | CCTCGGTG | | |
| A022703 | TCTAAGAAGATGA | TGTCCTTCAGGG | | 50-51 |
| | TGCAGACCC | ATAGTGCC | | |
| Cdc212 | GGCTTCAGCCTCA | AAAACAACCAA | 101 | 52-53 |
| | AGTTCTG | GTTGCCCTG | | |
| Cdc212 | GGCACTGAAATGA | AACAATTCAAGC | 265 | 54-55 |
| | CCTGGAT | AACCTCGG | | |
| Cdc212 | CTGTTCCTTCCCA | TTCAGTCACGCA | 225 | 56-57 |
| | GACTCCA | AACCTGAG | | |
| Cot | GCCCAGGACTTTG | GGTAACCTGCAG | 284 | 58-59 |
| | TCACTGT | CTCCACTC | | |
| Cot | GGGACATGCTCTT | GAACAAAGCCG | 277 | 60-61 |
| | GGTTCAT | GGTGATTTA | | |
| Cot | GCCCTCAGTTCTC | GGCAGAGAAGA | 110 | 62-63 |
| | CTAGCCT | CTGGTGGAG | | |
| Cot | CCCAGACTTAGCG | AGCAGAGACCTT | 277 | 64-65 |
| | TCTCAGG | TGGACTCG | | |
| Cot | GAAGGCTGAGTGA | TTGCACGAGGAG | 276 | 66-67 |
| | GTCCCAG | AAGGTTTT | | |
| Cot | GATGCCAACGAGA | AGAAGCCAAAA | 247 | 68-69 |
| | CCTGAAT | CCCTCACCT | | |
| Cot | AAAAAGCCCTGCA | ATTCAGGTCTCG | 107 | 70-71 |
| | AGAACTT | TTGGCATC | | |
| D18346 | TGTCCGCAGTGTG | ATGTCCAGGGTA | 165 | 72-73 |
| | GAAACTA | GAGAGCCC | | |
| D18402 | GGAGTTCTCCTAC | GAGGCTCTGAGC | 167 | 74-75 |
| | CCTGGCT | AGTGTCAA | | |
| D4Bir1 | GCGATGTTGTTG | CAGTGTCTTTCC | | 76-77 |
| | CG | ACATTT | | |
| D4Ertd296e | AGGCATATTGTAT | CCGGATGACTCT | 201 | 78-79 |
| | AATAAATTTGTA | ACTTGAC | | |
| | GT | | | |
| D4Hrb1 | GCTGTTTATGGGG | AATTTCTGAAGC | 194 | 80-81 |
| | TCGAGAA | AGGGGGAT | | |
| D4Hrb1 | TCCCCCTGCTTCA | AGGGGGATGATT | 192 | 82-83 |
| | GAAATTA | GTGAGTGA | | |
| D4mit313 | CTTCTTTAATCAAT | GGGCACATATGA | 196 | 84-85 |
| | CTCTGTCTCTGTG | ACCTCCTG | | |
| D4mit344 | CCAAACTCTTAGC | ACACAGAAGAC | 187 | 86-87 |
| | TTCTTCA | ACTGAAGAAC | | |
| D4Mit51 | CAGTTGTTAGAAG | AGGTGCATATAC | 123 | 88-89 |
| | CAGGATCCC | CTGGGATACTC | | |
| D4Mit59 | AGAGTTTGGTCTC | TATCCAACACAT | 108 | 90-91 |
| | TTCCCCTG | TTATGTCTGCG | | |
| D4Mit59 | GCCAGTGTGCTGA | AGGGACCTGGA | 119 | 92-93 |
| | AAGACTG | GACATCCTT | | |
| D4Nds16 | CTGTAGGCTGCTT | TGCCCCTTCAGC | | 94-95 |
| | TTATCTTTTG | ACATGCCA | | |
| D4smh6b | TGCAGTGTGACAT | GGAAAGCCAGG | 118 | 96-97 |
| | GTGCATAGAT | CTACGCAGAA | | |
| D4smh6b | CTGTAGGCTGCTT | TGCCCCTTCAGC | 102 | 98-99 |
| | TTATCTTTTG | ACATGCCA | | |
| D4smh6b | TAGTGTGGTTCCT | CGGTCTACATAG | 181 | 100-101 |
| | GACTAACCT | TGAGTGATTC | | |
| D4smh6b | AAAAGCATCCTGC | GGGTTATACAGA | 83 | 102-103 |
| | ATCCTTCTG | GAAACCCTGT | | |
| D4Xrf215@ | TTCCAAGCTCACA | GTGCTGCTCTGC | 124 | 104-105 |
| | CATCAGC | ATTGAGTG | | |
| D4Xr1243@ | GACAGTGTGGGAG | CCCAAGGCATAG | 203 | 106-107 |
| | AATCCGT | GTCACAAT | | |
| D4Xrf243@ | ATTGTGACCTATG | CGAAGGACCGTC | 105 | 108-109 |
| | CCTTGGG | ATCTGAGT | | |
| D4Xrf472@ | GGCTTTGATGTGA | AGCTCCTCATCG | 245 | 110-111 |
| | AAAAGGC | CTCATGTT | | |
| D4X | | | | |
| rf@472 | TGGAACATCTCTG | GGCTCTCATTGC | 193 | 112-113 |
| | TCGGAAG | CACCTTTA | | |
| D4X497@ | CCAGAGAACAGG | GTGCTGGATACA | 119 | 114-115 |
| | AGACCTGC | CTGGCAGA | | |
| D4X | | | | |
| rf@497 | GCGAGACGAGTG | ACACTGAAACCT | 129 | 116-117 |
| | GGTAGTTC | CGCTTGCT | | |
| D4X | | | | |
| rf@497 | AGCAAGCGAGGTT | ACGGGGCTTGAT | 204 | 118-119 |
| | TCAGTGT | CCTTTTAT | | |
| Dshv4 | AAGTTCATGGGCC | TACTAGCTACCC | 100-300 | 120-121 |
| | TCACCACCTGTC | TTCACATACC | | |
| Dshv5@ | ACCTAGCCACTGT | ACAGAAGCAGC | 100-300 | 122-123 |
| | CTCAGTCT | ATTTACACAG | | |
| Gnb1 | TGGGACAGCTTCC | AATGGGAATTGT | 213 | 124-125 |
| | TCAAGAT | GCTCTTGG | | |
| Gnb1 | GGGCATCTGGCAA | AGATAACCTGTG | 281 | 126-127 |
| | AGATTTA | TGTCCCGC | | |
| Gnb1 | GATGTCCGAGAAG | TGTCAGCTTTGA | 277 | 128-129 |
| | GGATGTG | GTGCATCC | | |
| Gnb1 | ACATGCAGGCTGT | TGTCAGCTTTGA | 166 | 130-131 |
| | TTGACCT | GTGCATCC | | |
| K00231 | GTGCTCTGCAGAC | GAGCCATTTTGA | 154 | 132-133 |
| | AAACCAA | CCCTTAAA | | |
| K00231 | TTTCAGGGTCAAA | TCGACAGCAACT | | 134-135 |
| | ATGGCTC | GTGCG | | |
| K00954 | GGTGAGAGTGGG | CCCGGGTGAGTT | 237 | 136-137 |
| | GAGATGAA | TAAGAACC | | |
| k00954 | GGTGAGAGTGGG | AGGTTAGGCCCA | 296 | 138-139 |
| | GAGATGAA | ATTTCCTG | | |
| k00954 | CCAGGGTTGCTGT | CAGGTTAGGCCC | 237 | 140-141 |
| | ACTGAGA | AATTTCCT | | |
| K01153 | GGTCAGAGTCCTT | TCCAACTTCACA | 124 | 142-143 |
| | CCTTCCC | GGAAACCC | | |
| K01153 | TTTCCTGTGAAGT | CACCCATATGGC | 213 | 144-145 |
| | TGGAGGG | AAACATCA | | |
| K01153 | GGTCAGAGTCCTT | TCCAACTTCACA | 125 | 146-147 |
| | CCTTCCC | GGAAACCC | | |
| K01153 | TGATGTTTGCCAT | GCTTGCTGCTTC | 181 | 148-149 |
| | ATGGGTG | CGATATGT | | |
| K01599 | GGAAAAGGGAGT | GAGCCGCCTAAC | 166 | 150-151 |
| | CGCCATA | TCTCACAC | | |
| K01599 | AGGGGATAACCTG | ACAAAATTGCTC | 110 | 152-153 |
| | CATAGG | ATTTGCCC | | |
| M-05262@ | CCATCCCCACTAG | GTCCCCTTTGTC | 169 | 154-155 |
| | CCAGATA | ACAGCAAG | | |
| M107-H01 | TGAGCACAGGATA | AAAAGAACACC | 217 | 156-157 |
| | GCTCCAC | TGTTTGGGG | | |
| M111-B04 | TAAACCTCGGCTG | CCCTCAGTGACT | 267 | 158-159 |
| | TGTGAG | TCCTGTGA | | |
| M134-C06 | CAAAACCACATGG | GCCCTATTGCCA | 264 | 160-161 |
| | TTACCGA | AATGACTT | | |
| M134-G01 | GGCAGAAAGGAA | CACATTAGCCAT | 161 | 162-163 |
| | TCAGAAGC | TGTCCTGG | | |
| M136-B01 | TCCTTTATGTCCA | CATGGTCTGTGA | 164 | 164-165 |
| | ACAGCCA | TGTGACCA | | |
| M156-H05 | ATACCCTTGGTGA | GCTGTCAAATGA | 139 | 166-167 |
| | GAGCAGG | GAAAGGCA | | |
| M184-B03 | TATTTCATGCTGG | AGAGAAAAACA | 89 | 168-169 |
| | GACCAAA | GTGGGGGTG | | |
| Mmp23 | CGGGTCCTCTCTT | CTACATTTCCCT | 297 | 170-171 |
| | CACCATA | GAGCTGCC | | |
| Mmp23 | GTTGACCATGTCG | CCACCTCACGGA | 111 | 172-173 |
| | GTAACCC | AACTGAAT | | |
| Mmp23 | GGTGTTTGGCTCA | GATGCACACACA | 197 | 174-175 |
| | CAAACCT | AAAATCCG | | |
| Mmp23 | ATCACCCACCAGA | ACCCTCCAGGAG | 255 | 176-177 |
| | ACGAAAA | TAGGTGCT | | |
| PCEE | GATGAGACAGTGG | TTGTCAATAGCA | 154 | 178-179 |
| | GCAAGGT | CCAAGCCA | | |
| PCEE | GCCTTAATAGCCC | GCACTCAGCATT | 194 | 180-181 |
| | CCTTGTT | GCACAGAT | | |
| PCEE | GGACGGACAATTC | CTATCACACCTC | 142 | 182-183 |
| | TGGAAAA | CGATGCCT | | |
| PCEE | CAAGCTGGTAGAA | TCTTTGGAGAAG | 209 | 184-185 |
| | TCCCCAA | CAGACCGT | | |
| Pkcz | TACAGCATATGCA | ATTCCTCAGGGC | 294 | 186-187 |
| | TGCCAGG | ATTACACG | | |
| Pkcz | GCAATCTCTTGTG | ATTCCTCAGGGC | 188 | 188-189 |
| | TCCAGGC | ATTACACG | | |
| Pkcz | TACAGCATATGCA | GGCCTGGACACA | 127 | 190-191 |
| | TGCCAGG | AGAGATTG | | |
| Pkcz | AAGTGGGTGGACA | CAGCTTCCTCCA | 201 | 192-193 |
| | GTGAAGG | TCTTCTGG | | |
| Pkcz | AGAGCCTCCAGTA | TCGTGGACAAGC | 297 | 194-195 |
| | GATGGCA | TCCTTCTT | | |
| Pkcz | CATCGAGTATGTC | TTGTCCAGTTTT | 156 | 196-197 |
| | AATGGCG | AGGTCCCG | | |
| Pkcz | CAGACTGGGTTTT | GTCAAAGTTGTC | 132 | 198-199 |
| | CCGACAT | CAGGCCAT | | |
| Pkcz | AGGACGGACCCCA | TGTCTCGCACTT | 130 | 200-201 |
| | AGATG | CCTCACAG | | |
| Pkcz | CCAGAAGATGGA | TCTACTGGAGGC | 151 | 202-203 |
| | GGAAGCTG | TCTTGGGA | | |
| Pkcz | GAAAAACGACCA | GATCTCAGCAGC | 265 | 204-205 |
| | GATTTACG | ATAGAACC | | |
| Pkcz | ACACATTAAGCTG | CAAACATAAGG | 164 | 206-207 |
| | ACGGACT | ACACCCAGT | | |
| Pkcz | ACTGGGTGTCCTT | CCTCTCTTTGGG | 193 | 208-209 |
| | ATGTTTG | ATCCTTAT | | |
| Pkcz | GTCATAAAGAGGA | GCTCTGTCTAGA | 252 | 210-211 |
| | TCGACCA | AGTGCCTG | | |
| Pkcz | ACCAAGACCGAA | GGCATTACACGC | 223 | 212-213 |
| | GAGGGG | TAACTTTTCC | | |
| R74924 | AGTGCCACCAACC | AAGTGCCTGCAG | 165 | 214-215 |
| | TGGTAAG | GGATGC | | |
| R74924 | TGCTTTGGTGAGC | AGGGACACCCTT | 103 | 216-217 |
| | AATGTTT | ACCAGGTT | | |
| R74924 | CTGATGCTTTGGT | GGGACACCCTTA | | 218-219 |
| | GAGCAAT | CCAGGTT | | |
| R75150 | ACAGGACAAATGC | GTGGTAAAGAA | 217 | 220-221 |
| | TGGGTTG | CGCTTGGCT | | |
| R75150 | GGTATCTCACTTG | AAGAACGCTTGG | | 222-223 |
| | GTAGGAACCTC | CTGGC | | |
| RER1 (1) | GCCGATCCTGGTG | ACAATGGCTCAA | | 224-225 |
| | ATGTACT | AACCGTTC | | |
| RER1 (2) | GCCTTGGGAATTT | AGTACATCACCA | | 226-227 |
| | ACCACCT | GGATCGGC | | |
| RER1 | TAAAAGGCCATGC | AGAGCTCTGTGG | | 228-229 |
| | GATAAGC | GGTTCTCA | | |
| RER1 | GAAGGGGACAGT | TCCATCAAGGAA | | 230-231 |
| | GTTGGAGA | GGATCCAC | | |
| Tp73 | GGTGGGTAATGAT | TGACGTGGAGG | 296-301 | 232-233 |
| | TGGACT | GAACTGCC | | |
| Tp73 | TGAGATCTGGTGC | GCCTGATCTAGG | 222-229 | 234-235 |
| | CCTCTCT | CTGGAAAA | | |
| Txgp1 | AGGCAGAAAGCA | CGACAGCACTTG | 138 | 236-237 |
| | GACAAGGA | TGACCACT | | |
| Txgp1 | CTGCAGATGTAGA | CTGTGGTGGATT | 269 | 238-239 |
| | CCAGGCA | GGACAGTG | | |
| Txgp1 | TTGCCTAACACTC | TATTAGGAGCAC | 244 | 240-241 |
| | CCAAACC | CACCAGGC | | |
| Txgp1 | ACCTGTCTTGTGG | CTGTGGTGGATT | | 242-243 |
| | GTGGAAG | GGACAGTG | | |
| U37351 | GTGGCTTGGTGCT | GGGGCTATTAAG | 160 | 244-245 |
| | ATTGACA | GCCATTTT | | |
| V2R2 | CAATTGAGGAATG | TGGCTTCATGTC | 170 | 246-247 |
| | GCTACCAA | CATTGTGT | | |
| V2R2 | CAGAACCACAAA | TCATGTTTGCTG | 163 | 248-249 |
| | GGTAAATTGC | TCCAGTTTG | | |
| TR1-like1 | GCCACCATGCTGG | TCACTCATGTTT | 2520 | 250-251 |
| (human) | GCCCTGCTGTCCT | CCCCTGATTTCC | | |
| | GGG | | | |
| T1-ike2 | CTGATTTCCTGTG | CATGCTGGCCTA | 244 | 252-253 |
| (human) | TTCCCGT | CTTCATCA | | |
| T1-like3 | GCCTTGCAGGTCA | TCACTCATGTTT | 2441 | 254-255 |
| (human) | GCTACGGTGCTAG | CCCCTGATTTCC | | |
| | CAT | | | |
| T1-like4 | AGGAAGCAGAGA | TCAGAACTGCCT | 274 | 256-257 |
| (human) | AAGGCCAG | CTGAGCTG | | |
| T1-like5 | TCTTCACGTACTG | ACTACAGCATCA | 175 | 258-259 |
| (human) | GGGGAAC | GCAGCAGG | | |
| T1-like6 | AAGCTGAAGAACT | TGGGCTACGACC | 211 | 260-261 |
| (human) | TCCCGGT | TCTTTGAT | | |
| h-Tr1like a | ATCTTCAGGCGCT | GTACGACCTGAA | | 262-263 |
| | CTGTCCT | GCTGTGGG | | |
| h-Tr1like b | ATCTTCAGGCGCT | GTACGACCTGAA | | 264-265 |
| | CTGTCC | GCTGTGGG | | |
| h-Tr1like c | ATCTTCAGGCGCT | GAGTACGACCTG | | 266-267 |
| | CTGTCC | AAGCTGTGG | | |
| h-Tr1like d | ATCTTCAGGCGCT | TACGACCTGAAG | | 268-269 |
| | CTGTCCT | CTGTGGG | | |
| h-Tr1like e | ATCTTCAGGCGCT | TACGACCTGAAG | | 270-271 |
| | CTGTCC | CTGTGGG | | |
| h-Tr1like | GCTGTCCCGATGG | ACCTTTTGTGGC | | 272-273 |
| | TGAAC | CAGGATG | | |
| h-Tr1like g | GCTGTCCCGATGG | CACCTTTTGTGG | | 274-275 |
| | TGAAC | CCAGGAT | | |
| h-Tr1like h | GCTGTCCCGATGG | CCTTTTGTGGCC | | 276-277 |
| | TGAAC | AGGATG | | |
| h-Tr1like j | CCTGAACCAGTGG | ACCTTTTGTGGC | | 278-279 |
| | GCTGT | CAGGATG | | |
| h-Tr1like j | CCTGAACCAGTGG | CACCTTTTGTGG | | 280-281 |
| | GCTGT | CCAGGAT | | |
| h-Tr1like k | TCATGTTTCCCCT | CATGCTGGCCTA | | 282-283 |
| | GATTTCC | CTTCATCA | | |
| h-Tr1like | ATGAGCAGGTAAC | TCATCACCTGGG | | 284-285 |
| | ACCTGGG | TCTCCTTT | | |
| h-Tr1like m | ATGAGCAGGTAAC | TTCATCACCTGG | | 286-287 |
| | ACCTGGG | GTCTCCTT | | |
| mTr1like-1A | TGGGTTGTGTTCT | CCTTTTTACAGT | | 288-289 |
| | CTGGTTG | CTGCCAGGT | | |
| mTr1like-1B | TGGGTTGTGTTCT | GATCCCCTTTTT | | 290-291 |
| | CTGGTTG | ACAGTCTGC | | |
| mTr1like-2A | ACGGGGTTGGTAC | CACCCATTGTTA | | 292-293 |
| | TGTGTGT | GTGCTGGA | | |
| mTr1like-2B | ACGGGGTTGGTAC | CACACACCCACC | | 294-295 |
| | TGTGTGT | CATTGTTA | | |
| mTr1like-3A | TGCATTGGCCAGA | CGGCTGGGCTAT | | 296-297 |
| | CTAGAAA | GACCTAT | | |
| mTr1like-3B | TGCATTGGCCAGA | CGGCTGGGCTAT | | 298-299 |
| | CTAGAAA | GACCTATT | | |
| mTr1like-4A | GTTCTGCAGCATG | GGCAGTTGTGAC | | 300-301 |
| | ATGTCGT | TCTGTTGC | | |
| mTr1like-4B | GTTCTGCAGCATG | CTGCAGGCAGTT | | 302-303 |
| | ATGTCGT | GTGACTCT | | |
| mTr1like-5A | CCATCCTTTTTGCC | TCTGGAGGAACA | | 304-305 |
| | TGTCTT | TGTGATGG | | |
| mTr1like-5B | CACCATCCTTTTT | GAACATGTGATG | | 306-307 |
| | GCCTGTC | GGGCAAC | | |
| mTr1like-6A | CAAAGCAGCAGG | AAATGTACTGGC | | 308-309 |
| | AGGAGTG | CAGGCAAC | | |
| mTr1like-6B | AGTGCTAGACCCA | AAATGTACTGGC | | 310-311 |
| | GCACCAG | CAGGCAAC | | |
| mTr1like-7A | GCACTGACCAGTC | GTCCCCAGAGAA | | 312-313 |
| | TGTCACC | AAGCACAG | | |
| mTr1like-7B | CAGTCTGTCACCA | CAGTGGTCCCCA | | 314-315 |
| | CCTCTGG | GAGAAAAG | | |
| mTr1like-8A | TACTATTCGGGGC | GCAGCACTATGT | | 316-317 |
| | TTGTTGG | GCCTGGTA | | |
| mTr1like-8B | TACTATTCGGGGC | GCCTGGTATTTG | | 318-319 |
| | TTGTTGG | ATCGCTTT | | |
| mTr1like-9A | GCTCAGCTAGGGA | CAGCTCAGGGAC | | 320-321 |
| | TGGAGAA | ACAATGAA | | |
| mTr1like-9B | TCCTACAGGCTAG | CAGCTCAGGGAC | | 322-323 |
| | GGCTCAG | ACAATGAA | | |
| mTr1like-10A | GGGACTGATGTGT | AGGCGTCCCAGG | | 324-325 |
| | GGCTTGT | AATAGAAG | | |
| mTr1like-10B | GGACTGATGTGTG | AGGCGTCCCAGG | | 326-327 |
| | GCTTGTTT | AATAGAAG | | |
| mTr1like-11A | TGTTTCTGTTCTGG | ATCTGCAGGCAG | | 328-329 |
| | TGGCTG | GATCAGAC | | |
| mTr1like-11B | CTCAGTGGTGGGT | ATCTGCAGGCAG | | 330-331 |
| | GACAGTG | GATCAGAC | | |
| Mutation 1 | ACACACAGTACCA | CCTGTGGTGATC | 182 | 332-333 |
| (mouse) | ACCCCGT | AAGAAGCA | | |
| Mutation 2 | TGCTTCTTGATCA | GCAACAGAGTC | 131 | 334-335 |
| (mouse) | CCACAGG | ACAACTGCC | | |
| Mutations 1 + 2 | ACACACAGTACCA | GCAACAGAGTC | 293 | 336-337 |
| (mouse) | ACCCCGT | ACAACTGCC | | |
| 34m15-T7 | GGGTTTATGTGGC | ACTCCATTTGCC | 118 | 338-339 |
| | AAGCACT | TTTTGTGG | | |
| 34m15-5P6 | CGCTACTTCGCTT | ATGATGACGTAC | 150 | 340-341 |
| | TTATCCG | GACGACGA | | |
| 37D20-T7 | GAAAACAATCGG | TGAAATTATCAC | 109 | 342-343 |
| | GGAGAAGTC | ACGCCAGG | | |
| 37D20-T7 (3)* | AGTGAGAGGCCCA | GATCTGATGCCC | 247 | 344-345 |
| | GTCTCAA | TCTTCTGC | | |
| 37D20-SP6 | GCTAGCCTTGAAG | TGAACAGCATGC | 122 | 346-347 |
| | CCAACAC | TTACCCAG | | |
| 49O2-T7 | TCCCTAGAGGCCT | TCGTCTCGGAGC | 169 | 348-349 |
| | GTCTGTC | CTCTTCTA | | |
| 49O2-SP6 | GATAGTCCCTTAG | GCCATAGCTCCT | 218 | 350-351 |
| | CCAGCCC | CACTGCTC | | |
| 73B10-T7 | CAGAGTGGGCTCT | TTGTGTTCAGAT | 237 | 352-353 |
| | GGTCTTC | GCTCCTGC | | |
| 73B10-SP6 | TTATTTCTGTGCTA | ATCAAGTCAACG | 267 | 354-355 |
| | GCCGCC | TCCCCAAG | | |
| 75M14 | ACCTGGCCTGTGC | GCACCAACCCTA | 233 | 356-357 |
| | TAATCTC | AGAAAGCA | | |
| 85G18 | TCAGGCTAACCTC | AAAGAAAAGAA | 113 | 358-359 |
| | AAACTCACA | AAGAAAAAGTC | | |
| | AGACA | | | |
| 118E21-T7 | CCCAGAACTCCAT | CCCAACCTGTGG | 185 | 360-361 |
| | CCTCAAA | TCAGCTAT | | |
| 118E21-SP6 | GGGGCAGGTGGGT | CAAAAGCCCAA | 271 | 362-363 |
| | AATAAGT | CTCCTTGAG | | |
| 130A12-T7* | GCTCAGTGGGTAA | CTACCCTGCCGC | 242 | 364-365 |
| | GAGCACC | TAATCTCA | | |
| 130A12-SP6 | CAGTTAGCACCCC | TCTGCACCTCTG | 114 | 366-367 |
| | ACCCTAA | TTCACCTG | | |
| 138D7-T7 | ACCTCTAGGGTTT | CCTCAGGTAGTG | 199 | 368-369 |
| | ACGGGGA | CAAGCTCC | | |
| 139J18-T7 | TCAGTTACCAAGG | ATAGGTTGTCAC | 122 | 370-371 |
| | GTTTCGG | AGGCCAGG | | |
| 139J18-SP6 | TCAGTTACCAAGG | ATAGGTTGTCAC | 122 | 372-373 |
| | GTTTCGG | AGGCCAGG | | |
| 147a15-T7* | GTGGTTGCTGGGA | CAAGCAACCAA | 101 | 374-375 |
| | TTTGAAC | ACAACCAAA | | |
| 147A15-SP6 | TCCGGAGGACCAT | CACAGTCCCAGT | 249 | 376-377 |
| | AAATCTG | CATTCCCT | | |
| 151E4-T7 | GTCCCAAAAGCTA | TCATGAGCCACC | 240 | 378-379 |
| | GCACAGG | ATGTGATT | | |
| 151E4-SP6 | GACCTTCGGAAGA | AGTGTGTGTCGC | 223 | 380-381 |
| | GCAGTTG | CATATCCA | | |
| 152O3-17 | CCTACTCTCTCTCC | GGAAAATGTTTG | 142 | 382-383 |
| | CCGCTT | GCCTTGAA | | |
| 152O3-SP6 | CTGGAGTGAAAGG | AGGCGGCACCAT | 537 | 384-385 |
| | CAGGAAG | ATGAATAA | | |
| 153B21SP6 | TGAGAGTGGGAAT | GGATGTAATTGG | 202 | 386-387 |
| | TCTGTTCA | TGGCAAGG | | |
| 153B21T7 | CTGTTGGAGGAGG | TGCTTGTATGTT | 113 | 388-389 |
| | TGGCCTA | TTTCCTCGT | | |
| 159J195P6 | TGAGAGTGCCCTC | GAACCCCTGACC | 200 | 390-391 |
| | CTCTTTG | CCAGAC | | |
| 159J19T7 | TGAAGTGCAGATT | GTTTTGGGGTGG | 213 | 392-393 |
| | TTTACATGG | AAAAGGAT | | |
| 189M12SP6* | CCGTCGACATTTA | GATACTGGGGTG | 189 | 394-395 |
| | GGTGACA | GTGGGTAA | | |
| 227G4-SP6 | CCGTCGACATTTA | CGTCCCAGCTGT | 219 | 396-397 |
| | GGTGACA | GTAACTGA | | |
| 227G4-T7* | GGAAGCAAATGCT | TATCCCTAGCCC | 243 | 398-399 |
| | CCACTAAA | CTTGTGTG | | |
| 236C12-SP6 | CCGTCGACATTTA | GGGTCCTGTTGG | 209 | 400-401 |
| | GGTGACA | TAGTGACC | | |
| 238O5T7 | TATAAGCAGCCCC | CAGGCCAGACA | 244 | 402-403 |
| | TCATTGG | CTGCTTACA | | |
| 238O55P6 | CCTTGGGATCTGG | TGGGTTTAGAGT | 251 | 404-405 |
| | TGTGACT | ACGGCTGG | | |
| 24718-T7 | ACCCATTTCCTAA | ATCTCTCCAGCC | 177 | 406-407 |
| | TCCCCTG | CCTCTCAG | | |
| 280G12-T7* | GGGCTGGGAATTG | TGAATCCCTTAC | 420 | 408-409 |
| | AACCTAT | AGCCTTGC | | |
| 280G12-SP6 | GCCCCATAAAATC | GCTCCGGAAGGC | 233 | 410-411 |
| | CACTCCT | TAGAAGAT | | |
| 284D21-T7 | GGTTTGGGAGTGT | ACTCAGTTGGCC | 138 | 412-413 |
| | ATAGGCAA | TCTCCTCA | | |
| 284D21-SP6 | ACAGAAATCCCTC | TCAGTGTGGACC | 105 | 414-415 |
| | ATGCGA | AGAAAGTCC | | |
| 298E4 | TCTGCAAGTCAGC | ACTCATAAGGGT | 100 | 416-417 |
| | TCTTGATAA | CAAGCTGTCTG | | |
| 298e4-T7 (3)* | TCTCCCCTTTTACC | GCAAGGAGTCA | 180 | 418-419 |
| | ACTCCC | AAAACAGCA | | |
| 307E5 | GCTAGTTGGGGAA | ACTGCAAATGTC | 149 | 420-421 |
| | CAAACCA | CAACTCCA | | |
| 338N4-T7 | CAGTTACACAGCT | GCAAGAGCCTA | 245 | 422-423 |
| | GGGACGA | GCAATCCAC | | |
| 338N4-SP6 | CAGTTTAGCACCC | TCTGCACCTCTG | 115 | 424-425 |
| | CACCCTA | TTCACCTG | | |
| 348P19-SP6 | GGGTTCCACTTGA | TGGTCTGTTTCC | 227 | 426-427 |
| | TGCTGAT | TGGAGCTT | | |
| 350D2-T7* | TGTAGGGAATGTT | ACATGGAACAG | 295 | 428-429 |
| | TCTGCACC | GATTCTGGC | | |
| 350D2-SP6 | GCAGGCAAACAG | ATGGGGGATCCC | 217 | 430-431 |
| | ACAGACAA | TTACTGAC | | |
| 360M12-T7 | CGGTCAGGAGTAG | CAGCAGCTGATA | 123 | 432-433 |
| | TGTGGGT | TTGAGGCA | | |
| 360M12-SP6 | AATGATGAAGTGT | CAACAGAACTCA | 100 | 434-435 |
| | CAGCCTCAG | AAGCCTGG | | |
| 382A8-SP6 | AGCAGGCACAGGT | AAGAACAGGAC | 202 | 436-437 |
| | CTCTTGT | AGTGGTGGG | | |
| 382A8-SP6 (2) | CAGCGATTGGCTC | GGGGCTTCCTTT | 531 | 438-439 |
| | TTCTCTT | CTGAGGTA | | |
| 386N4-T7 | AGCTCAGGTCCAG | ATTTTCCCCTCC | 107 | 440-441 |
| | CTTGGTA | TGCTTCTC | | |
| 386N4-5P6 | CCAAGCCTCTGCT | TGAGGGTGGAG | 109 | 442-443 |
| | GGTTATC | AATGGAAAG | | |
| 387-17 | GCCCCATAAAATC | TTGCCTAACACT | 214 | 444-445 |
| | CACTCCT | CCCAAACC | | |
| 387-SP6 | CAGTTACACAGCT | GCAAGAGCCTA | 245 | 446-447 |
| | GGGACGA | GCAATCCAC | | |
| 388I1 | CAGCACCTTCCTC | TGTCTCCAGAGG | 137 | 448-449 |
| | TGGTCTC | TTCTGCCT | | |
| 399I12-17 | TGGTGGTGTAATA | TCTTTAATTTTT | 102 | 450-451 |
| | CTATTCCTTTGCA | GGCTTTTTGATA | | |
| 399I12-SP6 | CAGCTGTGTGCAT | CATCATGAAGAC | 106 | 452-453 |
| | GTTGACC | TCAGGGCA | | |
| 415A22SP6 | GTCCACACCTGGC | CAGCACTCAGTG | 199 | 454-455 |
| | TTTTGTT | AGGTTCCA | | |
| 415G24SP6 | ATGTAATGGAAGG | CAGCACTCAGTG | 113 | 456-457 |
| | GCTGCTG | AGGTTCCA | | |
| 417B22-SP6 | AAACAGGCATGA | GGGTATCATTGT | 116 | 458-459 |
| | AACTCAGGA | CACCTCCA | | |
| 436P10-T7 | CACAGGCCAAGTT | CAGGGGACCTTC | 115 | 460-461 |
| | GTTGTTG | TGAATGAT | | |
| 438C18-T7 | AGCTCAGGTCCAG | ACCACAAAATTT | 115 | 462-463 |
| | CTTGGTA | TCCCCTCC | | |
| 438C18-SP6 | CGGGACCTAAAAC | TGGGGACAGTTA | 254 | 464-465 |
| | TGGACAA | CCAGGAAG | | |
| 457N22-T7 | CCGGAGGACCATA | CCTCAAAAACAA | 129 | 466-467 |
| | AATCTGA | GCCTGAGC | | |
| 457N22-SP6 | CCTTCAGAAATGT | TCCTGAGTTCAA | 252 | 468-469 |
| | GTTTGGACA | ATCCCAGC | | |
| 472O18 | CTTTCCATTCTCCA | AGGTCCTAGGGA | 260 | 470-471 |
| | CCCTCA | GAGGTCCA | | |
| D4Mon1 | AGGCCTACCCAAG | GCAGTGAGCTGC | 201 | 472-473 |
| | GACATCT | AGAGTTTG | | |
| D4Mon2 | AGACACCCTAGGT | TGATCTTTCCAA | 151 | 474-475 |
| | CCTGCTG | ACGCATAAGA | | |
| D4Mon3 | GCAAGCAACCTGA | GCTTACGATGGT | 188 | 476-477 |
| | ACATGAA | CGTGAGGT | | |
| D4Mon4 | ACATGCCTGCCTA | GGAACCTGTTTT | 197 | 478-479 |
| | TCTTTGC | CCATGGTG | | |
| D4Mon5 | ACCTTGTTCCTGG | TAGCTGGGACGT | 200 | 480-481 |
| | TGTGAGC | GGTATGGT | | |
| D4Mon6 | CCATGGGAGACCA | TGAGTGTCCTCT | 206 | 482-483 |
| | GAAGGTA | GCCTGATG | | |
| D4Mon7 | GCGCTGACATCCT | CCCACTATGGTC | 187 | 484-485 |
| | CCTATGT | CCAGAGAA | | |
| D4Mon8 | TTGCACGTCTTTG | AAAGGGGAATA | 219 | 486-487 |
| | TTTCGAG | GACCTGAGTAG | | |
| | AA | | | |
| D4Mon9 | CCAAGAGTCAGCC | GGACAGGTAGCT | 200 | 488-489 |
| | TTGGAGT | CACCCAAC | | |
| Tr1likeu1cDNA | TGCCAGCTTTGGC | TTCATTGTGTCC | | 490-491 |
| mouse | TATCAT | CTGAGCTG | | |
| Tr1likeu2cDNA | AGCTTTGGCTATC | ACCACCGCCACT | | 492-493 |
| mouse | ATGGGTCTCAG | GTTCTCATCT | | |
| Tr1like_A1 | TGTGGGGGAAGA | TGATGTGTGGCT | 5935 | 494-495 |
| (mouse) | ACATAGAA | TGTTTCTCTT | | |
| Tr1like_A2 | ATAGGTGGGGAG | TGATGTGTGGCT | 5903 | 496-497 |
| (mouse) | GGAGCTAA | TGTTTCTCTT | | |
| TR1 like-2 | TGTGCCTGTCACA | CATGCTAGCACC | | 498-499 |
| (human) | GCAACTT | GTAGCTGA | | |
| TR1 like-3 | GGAGACCTTCCCC | GCTGTAGTTGAA | | 500-501 |
| (human) | TCCTTCT | GAGGGCGT | | |
| TR1 like-4 | GTGCTTGGCTTCC | CAGGTCGTACTC | | 502-503 |
| (human) | TCCAG | CATGTCCA | | |
| TR1 like-5 | TGGAGTACGACCT | ACTCATCCTGGC | | 504-505 |
| (human) | GAAGCTG | CACAAAAG | | |
| TR1 like-6 | GAACAGGAGGAC | CTTTTGTGGCCA | | 506-507 |
| (human) | GCTGAGG | GGATGAGT | | |
| TR1 like-7 | TCACCTCACCTGG | GTACGACCTGAA | | 508-509 |
| (human) | TTGTCAG | GCTGTGGG | | |
| TR1 like-8 | GGCTGAGATCACA | CCGTGCCTGTTG | | 510-511 |
| (human) | GGGTTGGGTCACT | GAAGTTGCCTCT | | |
| | C | GCC | | |
| 118e21-0 | AATTCCCAGCAAC | CAGACACTCCAG | 585 | 512-513 |
| | CACTCAC | AAGAGGGC | | |
| 118e21-1 | TGACTGCTCTTCC | TTTGTGGAATAG | 588 | 514-515 |
| | GAAGGTT | CCAAAGCC | | |
| 118-21-2 | TCTCTCCTCTCTTC | AGCAGGGTGCAT | 551 | 516-517 |
| | TCCCCC | CACCTTAT | | |
| 118e21-3 | TAGGAGTGCCCCA | TCATTGTACCCA | 518 | 518-519 |
| | TAGGTTG | GCCAGTCA | | |
| 118e21-4 | AGGACTGAGCCTG | CTGGGCGTTTTG | 552 | 520-521 |
| | GATGAGA | TTTTGTTT | | |
| 118e21-5 | CTTCCTCCTGCAG | ACCCTGCTACAA | 546 | 522-523 |
| | CTACCAC | CGCAGACT | | |
| 118e21-6 | TCCAACCTTGACA | AGCCAGGGCTAC | 584 | 524-525 |
| | CCCATTT | ACAGAGAA | | |
| 139J18T7 (1) | CTGCTTTTCCTCA | ATTCGCCGTTAG | | 526-527 |
| | GCAACTG | AAGCTAGG | | |
| 139J18T7 (2) | AACTGTACGTGGC | ATTCGCCGTTAG | | 528-529 |
| | TGCTGGT | AAGCTAGG | | |
| Agrin (CA) n | GCCAGGTGACCCT | GAGAGATGGCA | 271 | 530-531 |
| | TATGAAA | GACAGAGGC | | |
| Agrin (TG) n | AGCTCTCTGTCCC | TGCCAACCACTA | 157 | 532-533 |
| | TGGTGAA | GCCTCTCT | | |
| repeat1 | CTGAACCCTCCAC | AGCCAGGGCTAC | 205 | 534-535 |
| | TCTCCTG | ACAGAGAA | | |
| repeat2 | AGCCAGGGCTACA | ACCCTGCTACAA | 153 | 536-537 |
| | CAGAGAA | CGCAGACT | | |
| repeat3 | GCAAGTTTCAGGA | CCCCAGAACCAG | 166 | 538-539 |
| | GCTAGGG | AGACCATA | | |
| repeat4 | CTAGGGGACTCTG | CAAGACACCCA | 195 | 540-541 |
| | CCAAGTG | GTCCCAACT | | |
| repeat5 | TACTTCCCCTTTCC | TCCTTGGTGCTT | 232 | 542-543 |
| | CGAACT | ACCCTCAC | | |
| repeat6 | TGTTCCTGAGTTC | ATTCCCAGCAAC | 269 | 544-545 |
| | ACAACGC | TACATGGC | | |
| repeat7 | ACATGTCCACTGT | TGTCATGAGTTT | 246 | 546-547 |
| | GGCAAAA | GAGGCCAG | | |
| repeat8 | ATCAGACAGCCCA | TATGTGCCACCA | 206 | 548-549 |
| | CAACCTC | CACCTGTC | | |
| repeat9 | GCTCAAGGAAGG | TGCTCTTAACAT | 201 | 550-551 |
| | ACACACCT | TTTGAGCCAT | | |
| repeat10 | GCTCAGCCCCTGA | GGGATCTGCCTG | 111 | 552-553 |
| | ATCAATA | TCTTACCA | | |
| repeat11 | GGAAGGTAGGGC | GCTCCAAGATCT | 277 | 554-555 |
| | CTGGTAAT | GTGCGATT | | |
| repeat12 | TTAGCGTTAGGGT | GGAGACTACGG | 150 | 556-557 |
| | GAGGGTG | ACTTGTGGC | | |
| repeat13 | CAGTTCTTCCCGA | TTTCTGGGAACT | 174 | 558-559 |
| | AAACCAC | GAGATGGC | | |
| repeat14 | GTTGGGGCTGCTC | GCTGTGGCTCTC | 422 | 560-561 |
| | ATAGAAA | TTGGAGTT | | |
| repeat15 | CTCTGATTTCCCA | AAGAGGGAGCA | 152 | 562-563 |
| | CATGCCT | CTGAGGACA | | |
| repeat16 | CAGCAGCAAATGA | GAGGCAGGCAG | 147 | 564-565 |
| | CCTTTCA | ATTTCTGAG | | |
| repeat17 | GTTTCACATGTTG | GGGACCTTTGGG | 131 | 566-567 |
| | TGGTGGC | ATAGCATT | | |
| repeat18 | TCAGACATCTCTG | TTCACTAAGTTG | 160 | 568-569 |
| | GCCTCCT | CCCAGGCT | | |
| repeat19 | TGCCTTTTTCTCAC | TTAGAAGCAGA | 250 | 570-571 |
| | ATTGTCTC | GGCAGAGGC | | |
| repeat20 | GACCTTTGGAAGA | TGGCAGCTCACA | 296 | 572-573 |
| | GCAGTCG | ATGTCTTT | | |
| SHANRU1 | GGTGTGGTGTAGG | TTTCAACTGCAA | 301 | 574-575 |
| | GGAAGAA | ACACAAACAG | | |
| SHANRU2 | AGGGCCAAGGAA | GCAAATATATAG | 203 | 576-577 |
| | GGAGAAT | GGTACCGAGCTG | | |
| SHANRU3 | CAGATTCTCCAGC | CTGTGTTTCCGC | 229 | 578-579 |
| | TGTCAGG | ACCAAGT | | |
| SHANRU4 | CTGCCCGTCCTTA | ACGCACGCTCAC | 289 | 580-581 |
| | TCTTCTG | TCATACAC | | |
| SHANRU5 | CAGCAGAGGTGAT | TTGTCACACAGT | 203 | 582-583 |
| | GGGTTCT | GGTTAAATGC | | |
| SHANRU6 | TAGAACCGTGGCT | CCGTAAGATAT | 201 | 584-585 |
| | GAGGACT | GAAAGAACTTG | | |
| | GA | | | |
| SHANRU7 | TAATCCTGGCTTA | TAGAAAGCACA | 240 | 586-587 |
| | GCGCTTG | GGGGACAGG | | |
| SHANRU8 | CCTTCCTCGTCTG | TTGGGACGTGAC | 232 | 588-589 |
| | AGCTGTT | CTGAGAAT | | |
| SHANRU9 | TATGTGTCTGGCC | GATGTGGGTGCA | 206 | 590-591 |
| | GTTGTTC | GGTGAAG | | |
| SHANRU10 | CCCCTTCTGGAGT | TCTAGGCAGGGC | 263 | 592-593 |
| | GTCTGAA | TACCTTTTT | | |
| SHANRU11 | GCTGAGCAGCCTC | ACCATGGCTTTT | 241 | 594-595 |
| | TAGCAA | CCCAGTAA | | |
| SHANRU12 | CTGTGCCTTTGGT | TGTGGCACTCTA | 261 | 596-597 |
| | GATCAGA | CGGCATAA | | |
| SHANRU13 | TGCATCACTATTA | AAGAATTTGCAA | 260 | 598-599 |
| | AGCCTCAACC | AGACTGTGAGA | | |
| SHANRU14 | AGCCAGCGCTACA | CTGGACCTTTGG | 199 | 600-601 |
| | CAGAGA | AAGAGCAG | | |
| SHANRU15 | GGTGGCTCAAACC | GAGGGCAATGA | 203 | 602-603 |
| | ATCCATA | GCAAAATGT | | |
| SHANRU16 | GGTCCTGTCTCTG | TAACACCCACAT | 201 | 604-605 |
| | GTTCAGG | CAGGCAAC | | |
| SHANRU17 | TTTCATTTCCTGGT | AAACACAGGCG | 198 | 606-607 |
| | GTTCCTTT | GAACGATAG | | |
| SHANRU18 | CTATCGTTCCGCC | AAGGAAGAGGA | 397 | 608-609 |
| | TGTGTTT | TGGAGAAAGA | | |
| SHANRU19 | CGGGTCTTAATGG | TCCTCCCCAGTT | 222 | 610-611 |
| | AGCAGAG | ACCTAGCA | | |
| SHANRU20 | CAGCAGGCAAGAT | GTCCCTCACCAG | 205 | 612-613 |
| | GACCTC | CCATGTTA | | |
| SHANRU21 | AGCCTGGGCTAAG | TATGGGCCAATG | 204 | 614-615 |
| | TTGTGTG | TTGTTCCT | | |
| SHANRU22 | ATGGTGGCTCACA | TTGTCCTCTGAT | 193 | 616-617 |
| | ACCATCT | TGCAGCAT | | |
| SHANRU23 | CTTGGGTCATCAG | AAGCTGCCCTGC | 301 | 618-619 |
| | GCTTTGT | TCTCTCTA | | |
| SHANRU24 | ATGCTCAGCCTGC | GCTGATAGCCCT | 198 | 620-621 |
| | TTTGTTT | GGGTTCTA | | |
| SHANRU25 | TGTACGCACAAAT | GAATCCACATTG | 222 | 622-623 |
| | TGACTTGC | CAAAGCCTA | | |
| SHANRU26 | CACAGGCAAATGA | CCAGACTTCTCC | 187 | 624-625 |
| | AGGGAAG | AGCTCTCC | | |
| SHANRU27 | TCCTCGAGAGGCT | TGCCTAGTCAAC | 237 | 626-627 |
| | CTAGGTTT | CACAGGAG | | |
| SHANRU28 | CCTGTGGTTGACT | GCCTGATAGCCT | 406 | 628-629 |
| | AGGCAGAA | GGAATACA | | |
| SHANRU29 | AAAGGGATGTGTG | CAAAACCCAACC | 195 | 630-631 |
| | GCGTAAG | TTCTCAGC | | |
| SHANRU30 | TGCACTGACCGTG | CGGTGTAGCTCT | 200 | 632-633 |
| | ATAGAGG | GGCTGTCT | | |
| SHANRU31 | CATCTCACCAACT | TTTCTGGGAACA | 418 | 634-635 |
| | CGCACTT | AAGAGGCTA | | |
| SHANRU32 | GAACCCAAGTGTT | TGGAAGCCCATC | 222 | 636-637 |
| | GGGGTAA | TGTCTCTT | | |
| SHANRU33 | AAATGCAAGTGGG | CCAGAAGAGGG | 187 | 638-639 |
| | TGCTTCT | CGTCAGAT | | |
| SHANRU34 | GGTGTGCACCACC | GGGAATTATCAG | 201 | 640-641 |
| | ATATTCA | CCAAAAAGC | | |
| SHANRU35 | GCCCAACTGAAAG | GGAAGGGGGAT | 263 | 642-643 |
| | CTCAACT | AACAATTGAA | | |
| SHANRU36 | TGCTAATTTCAAG | AGCTTGACACCT | 369 | 644-645 |
| | CACAGTGAGA | TGACAGCA | | |
| SHANRU37 | AACCTGCAGAGAG | CTCCAAGGGGA | 201 | 646-647 |
| | GAGACCA | GGACTCATT | | |
| SHANRU38 | TTCAATTGAGTTT | TGCAGGACCAA | 200 | 648-649 |
| | CTCTCCTCTGA | GAAGTAGGC | | |
| SHANRU39 | CGAGATCTGATGC | TGCTGAGAGCAG | 200 | 650-651 |
| | CCTCTTC | AAAAGGAA | | |

Although the foregoing invention has been described in some detail by way of illustrating and example for purposes of clarity of understanding, it will be obvious that certain changes and modifications may be practiced within the scope of the appended claims.

All publications, patents, and web sites are herein incorporated by reference in their entirety to the same extent as if each individual publication, patent, or web site was specifically and individually indicated to be incorporated by reference in its entirety.

What is claimed is:

1. An isolated polynucleotide comprising a sequence variation of SEQ ID. NO 1, wherein said variation is associated with sensing carbohydrates, other sweeteners, or ethanol.

2. An isolated polynucleotide comprising a sequence variation of SEQ ID. NO 2, wherein said variation is associated with sensing carbohydrates, other sweeteners, or ethanol.

3. An isolated polynucleotide comprising a sequence variation of SEQ ID. NO 4, wherein said variation is associated with altered sensation of carbohydrates, other sweeteners, or ethanol.

4. The polynucleotide of claim 1 wherein said variation is a missense mutation.

5. The polynucleotide of claim 4 wherein said variation is a nonsense mutation.

6. An isolated polypeptide comprising a variant form of SEQ ID. NO: 3, wherein said variant form is associated with altered preference for carbohydrates, other sweeteners, or ethanol.

7. An isolated polypeptide comprising a variant form of SEQ ID. NO 5, wherein said variant form is associated with altered preference for carbohydrates, other sweeteners, or ethanol.

8. An isolated polynucleotide having at least 8 contiguous nucleotides of the polynucleotides of any one of the claims 1-3 wherein said 8 contiguous nucleotides span said variation position.

9. An isolated polypeptide having at least four contiguous amino acids of the polypeptides of claims 6 or 7 wherein said four contiguous amino acids span said variation position.

10. An isolated polynucleotide wherein said polynucleotide is substantially identical to the polynucleotide of claim 8.

11. An isolated polypeptide wherein said polypeptide is substantially identical to the polypeptide of claim 9.

12. An isolated polynucleotide having a sequence which is complementary to the polynucleotide of claim 8 or 10.

13. A polynucleotide specific for the SAC1 locus wherein said polynucleotide hybridizes, under stringent conditions, to at least 8 contiguous nucleotides of the polynucleotide of claim 1, 2, 3, or 4.

14. The polynucleotide according to claim 13 wherein said polynucleotide is selected from the group consisting of SEQ ID. NOS 6-651 and homologous equivalents thereof.

15. A polynucleotide specific for the SAC1 locus wherein said polynucleotide that hybridizes, under stringent conditions, to at least 8 contiguous nucleotides of the polynucleotide of claim 3.

16. The polynucleotide of claim 15 wherein said polynucleotide is selected from the group consisting of SEQ ID. NOS 6-651 and homologous equivalents thereof.

17. A kit for the detection of the polynucleotide of any one of claims 1-5, 8, and 10 comprising a polynucleotide that hybridizes, under stringent conditions, to at least 12 contiguous nucleotides of the polynucleotide of any one of the claims 1-5, 8, and 10, and instructions relating to detection.

18. An isolated antibody which is immunoreactive to the polypeptide of claim 9 or 11.

19. A method for analyzing a biomolecule in a biological sample, wherein said method comprising:
a) altering SAC1 activity in a biological sample; and
b) measuring the activity.

20. A method for analyzing a polynucleotide in a biological sample comprising the steps of:
a) contacting a polynucleotide in a biological sample with a probe wherein said probe hybridizes to the polynucleotides of claim 8 or 10 to form a hybridization complex; and
b) detecting the hybridization complex.

21. A method for analyzing the expression of SAC1 comprising the steps of
a) contacting a biological sample with a probe wherein said probe comprises the polynucleotide of claim 8 or 10; and
b) detecting the expression of SAC1 mRNA transcript in said sample.

22. The method of claim 19 wherein said step of measuring is an enzymatic assay.

23. The method of claim 20 or 21 wherein said probe is immobilized on a solid support.

24. The method according to any one of the claims 19-23 wherein said sample is derived from blood.

25. The method according to any one of the claims 19-23 wherein said sample is derived from tongue.

26. The method according to any one of the claims 19-23 wherein said sample is derived from pancreas.

27. The method according to any one of the claims 19-23 wherein said sample is derived from a human.

28. A method for identifying susceptibility to obesity or diabetes which comprises comparing the nucleotide sequence of the suspected SAC1 allele with a wild type nucleotide sequence, wherein said difference between the suspected allele and the wild-type sequence identifies a sequence variation of the SAC1 nucleotide sequence.

29. An expression vector comprising the polynucleotide of claim 3, 8, or 10.

30. A host cell comprising the expression vector of claim 29.

31. A method of producing a polypeptide comprising culturing the cells of claim 30 and recovering the polypeptide from the host cell.

32. An isolated polypeptide produced according to claim 31.

33. A method for conducting a screening assay to identify a molecule which enhances or decreases the SAC1 activity comprising the steps of
a) contacting a biological sample with a molecule wherein said biological sample contains SAC1 activity; and
b) analyzing the SAC1 activity in said sample.

34. A pharmaceutical composition comprising
a) the polynucleotide of claim 8 or 10, the polypeptide of claim 9 or 11, the antibody of claim 18 or the molecule of claim 18; and
b) a suitable pharmaceutical carrier.

35. A method for treating or preventing obesity, diabetes, or alcoholism associated with expression of SAC1, wherein said method comprises administering to a subject an effective amount of the pharmaceutical composition of claim 34.

36. A transgenic animal that carries an altered SAC1 allele.

37. The transgenic animal of claim 36 is a knock out mouse.

38. The polypeptide of claim 6 or 7, wherein said polypeptide is 7-transmembrane G protein coupled receptor (7TM GPCR).
